(19) United States
(12) Patent Application Publication
(10) Pub. No.: US 2024/0131203 A1
(43) Pub. Date: Apr. 25, 2024

(54) FIBROBLAST ACTIVATION PROTEIN (FAP) INHIBITORS, FAP CONJUGATES, AND DIAGNOSTIC AND THERAPEUTIC USES THEREOF

(71) Applicants: Nuclidium AG, Basel (CH); University of Basel, Basel (CH)

(72) Inventors: Leila JAAFAR-THIEL, Erlangen (DE); Melpomeni FANI, Basel (CH); Jacopo MILLUL, Basel (CH); Francesco DE ROSE, Munich (DE)

(73) Assignees: Nuclidium AG, Basel (CH); University of Basel, Basel (CH)

(21) Appl. No.: 18/474,209

(22) Filed: Sep. 25, 2023

Related U.S. Application Data
(60) Provisional application No. 63/520,329, filed on Aug. 17, 2023.
(60) Provisional application No. 63/520,323, filed on Aug. 17, 2023.
(60) Provisional application No. 63/416,479, filed on Oct. 14, 2022.
(60) Provisional application No. 63/409,687, filed on Sep. 23, 2022.

(51) Int. Cl. A61K 51/04; C07F 1/08

(52) U.S. Cl. CPC A61K 51/0482; C07F 1/08; A61K 2121/00; A61K 2123/00

(57) ABSTRACT

The present disclosure relates to the field of fibroblast activation protein (FAP) inhibitors, conjugates comprising the novel FAP inhibitors, including radiotracers, for the imaging, diagnosis and treatment of conditions characterized by overexpression of FAP.

### 1. BACKGROUND

The present disclosure is directed to novel fibroblast activation protein (FAP) inhibitors and conjugates comprising the novel FAP inhibitors, including radiotracers, for the diagnosis and treatment of conditions characterized by expression of FAP.

In nuclear medicine, radiotracers are used for the diagnosis and therapy of various conditions and diseases. Radiotracers are compounds in which radionuclides are linked to targeting moieties that target specific organs, cells, or biomarkers in the human body.

Radiotracers can be used in targeted radionuclide therapy with the use of targeting moieties that selectively localize in malignant cells, tumors, or the microenvironments associated therewith, and with radionuclides selected to emit low-range highly ionizing radiation, e.g., α or β⁻ particles and Auger electrons. The combination of both the diagnosis and the treatment of a disease utilizing the same or similar biological targeting moieties which target a specific biomarker (e.g., a cell surface receptor) with different diagnostic and therapeutic radionuclides is called targeted theranostics. This approach overcomes the difficulty of quantifying the individual dose needed for the therapy through the diagnosis, rendering the treatment of the patient highly individualized. The theranostic approach is further improved using radionuclides of the same element, e.g., copper radionuclides, ⁶⁰Cu, ⁶¹Cu, ⁶²Cu, and ⁶⁴Cu as positron emitters in diagnostic imaging and ⁶⁷Cu as a β⁻ emitter in the radiotherapeutic, as the isotopically different radiotracers will bind identically to the biomarker.

FAP is a transmembrane glycoprotein expressed on activated fibroblasts such as cancer-associated fibroblasts (CAFs), a primary component of tumor microenvironment. Structurally, FAP is a type II transmembrane glycoprotein consisting of 760 amino acids. FAP is a serine protease, and unlike other members of the dipeptidyl peptidase (DPP) family, it has both endopeptidase and exopeptidase activity, which enable it to cleave gelatin and type I collagen and play an important role in extracellular matrix (ECM) remodeling. CAFs with FAP expression are found in various neoplasms, particularly epithelial cancers, and in malignancies with a strong desmoplastic reaction such as breast, colorectal, pancreatic, and lung cancer. Overall, a high degree of FAP expression is associated with tumor aggressiveness and poor prognosis (Cohen, S. J.; Alpaugh, R. K.; Palazzo, I.; Meropol, N.J.; Rogatko, A.; Xu, Z.; Hoffman, J. P.; Weiner, L. M.; Cheng, J. D. Fibroblast Activation Protein and Its Relationship to Clinical Outcome in Pancreatic Adenocarcinoma. Pancreas 2008, 37, 154-158). The negligible expression of FAP in normal healthy adult tissues makes it an attractive target for oncological imaging and therapy (Lindner, T.; Loktev, A.; Giesel, F.; Kratochwil, C.; Altmann, A.; Haberkorn, U. Targeting of Activated Fibroblasts for Imaging and Therapy. EJNMMI Radiopharm. Chem. 2019, 4, 16.).

FAP overexpression has been targeted with small-molecule FAP inhibitors (aka “FAPIs”) with an N-(4-quinolinoyl)-Gly-(2-cyanopyrrolidine) scaffold, first developed at the University of Antwerp (Hansen, K.; Heirbaut, L.; Cheng, J. D.; Joossens, J.; Ryabtsova, O.; Cos, P.; Maes, L.; Lambeir, A.-M.; De Meester, I.; Augustyns, K.; et al. Selective Inhibitors of Fibroblast Activation Protein (FAP) with a (4-Quinolinoyl)-Glycyl-2-Cyanopyrrolidine Scaffold. ACS Med. Chem. Lett. 2013, 4, 491-496; Jansen, K.; Heirbaut, L.; Verkerk, R.; Cheng, J. D.; Joossens, J.; Cos, P.; Maes, L.; Lambeir, A.-M.; De Meester, I.; Augustyns, K.; et al. Extended Structure-Activity Relationship and Pharmacokinetic Investigation of (4-Quinolinoyl)Glycyl-2-Cyanopyrrolidine Inhibitors of Fibroblast Activation Protein (FAP). J. Med. Chem. 2014, 57, 3053-3074). This scaffold was modified to develop FAPI-01 and FAPI-02 as the first quinoline-based FAPIs, which were radiolabeled with ¹²⁵I and ⁶⁸Ga/¹⁷⁷Lu, respectively. Further attempts at improving tumor retention led to the development of FAPI-46 (Loktev, A.; Lindner, T.; Burger, E.-M.; Altmann, A.; Giesel, F.; Kratochwil, C.; Debus, J.; Marme, F.; Jager, D.; Mier, W.; et al. Development of Fibroblast Activation Protein-Targeted Radiotracers with Improved Tumor Retention. J. Nucl. Med. 2019, 60, 1421-1429). WO 2019/154886 describes FAP inhibitors, FAP inhibitor-chelator constructs, radiolabeled FAP inhibitor-chelator constructs useful for diagnosis or treatment of diseases characterized by overexpression of FAP, e.g., cancer.

Because FAP overexpression is not limited to CAFs, the use of FAP inhibitors in combination with positron emission tomography-computed tomography (PET-CT) may find application in a wide range of non-oncological pathological states, e.g., inflammatory, infectious, and immune pathologies. FAP overexpression has also been associated with cardiovascular diseases, liver fibrosis and cirrhosis, arthritic disorders (e.g., rheumatoid arthritis), IgG4-related disease, pulmonary fibrosis and interstitial lung disease, Crohn's disease, tuberculosis, sarcoidosis, and periprosthetic joint infections (Chandekar, K. R.; Prashanth, A.; Vinjamuri, S.; Kumar, R. FAPI PET/CT Imaging—An Updated Review. Diagnostics 2023, 13, 2018).

The availability of a large portfolio of FAP inhibitors and radiotracers is essential for the development of nuclear medicine.

Accordingly, an object of the present disclosure is to provide compounds, compositions, and methods that fully or in part overcome one or more of the issues recognized in the prior art encompassing FAP inhibitors; conjugates comprising FAP inhibitors, including pharmaceuticals and radiotracers; and their use.

### 2. SUMMARY

The present disclosure relates to improved FAP inhibitors, conjugates comprising the improved FAP inhibitors, and their use in the diagnosis and treatment of various diseases characterized by overexpression of FAP.

In one aspect, compounds are provided herein, wherein the compound is of Formula I:

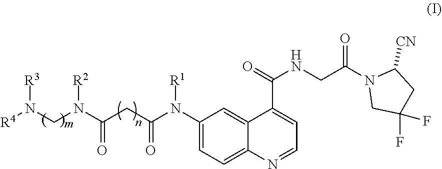

wherein:
R¹ is R^{a};
R² and R³ are each R^{a} or together form a C₂₋₉ heterocycle with the nitrogen atoms to which they are attached;
R⁴ is H, an amine protecting group, or -L-T;
R^{a}, independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl;
L is a bond or a divalent linker;
T comprises (a) a chelating moiety suitable for chelating a radionuclide, (b) an imaging agent, or (c) a drug;
n is an integer from 1 to 20; and
m is an integer from 1 to 20;
   or is a pharmaceutically acceptable salt thereof.

In another aspect, the compound is of Formula II:

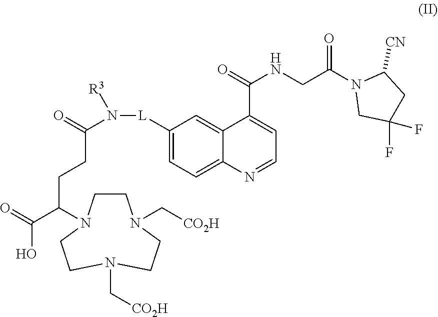

wherein:
R³ is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; or
R³, together with a moiety in L, form a C₂₋₉ heterocycle with the nitrogen atom(s) to which they are attached; and
L is a divalent linker, preferably up to 20 atoms in length;
   or is a pharmaceutically acceptable salt thereof.

In another aspect, the compound is of Formula III:

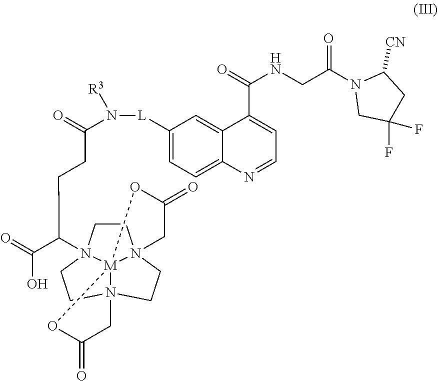

wherein: R³ is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; or
R³, together with a moiety in L, form a C₂₋₉ heterocycle with the nitrogen atom(s) to which they are attached;
L is a divalent linker, preferably up to 20 atoms in length; and
M is selected from ²²⁵Ac, ⁵¹Cr, ⁶⁶Ga, ⁶⁷Ga, ⁶⁸GA, [¹⁸F]AlF, ¹¹¹In, ^{113m}In, ^{52m}Mn, ^{99m}Tc, ¹⁸⁶Re, ¹⁸⁸Re, ¹³⁹La, ¹⁴⁰La, ¹⁷⁵Yb, ¹⁷⁹Yb, ¹⁵³Sm, ^{177m}Sn, ¹⁶⁶Ho, ⁸⁶Y, ⁸⁸Y, ⁹⁰Y, ¹⁴⁹Pm, ¹⁶⁵Dy, ¹⁶⁹Er, ¹⁷⁷Lu, ⁵²Fe, ⁴³Sc, ⁴⁴Sc, ⁴⁶Sc, ⁴⁷Sc, ¹⁴²Pr, ¹⁵⁷Gd, ¹⁵⁹Gd, ²¹²Bi, ²¹³Bi, ⁷²As, ⁷⁷As, ⁹⁷Ru, ¹⁰⁹Pd, ¹⁰⁵Rh, ^{101m}Rh, ¹¹⁹Sb, ¹⁹⁷Hg, ¹⁵¹Eu, ¹⁵³Eu, ¹⁶⁹Eu, ²⁰¹Tl, ¹⁴⁹Tb, ¹⁵²Tb, ¹⁵⁵Tb, ¹⁶¹Tb, ²⁰³Pb, ²¹²Pb, ¹⁵¹Pm, ¹⁵³Pm, ¹⁴²Pr, ¹⁴³Pr, ⁵⁵Co, ⁶⁰Cu, ⁶¹Cu, ⁶²Cu, ⁶⁴Cu, ⁶⁷Cu, ⁶²Zn, ¹⁸⁸Re, ¹⁹⁸Au, ¹⁹⁹Au, ²²⁷Th, ¹¹¹Ag, ¹⁹⁹Ag, ²¹¹At, ²²³Ra, ⁸⁸Zr, and ⁸⁹Zr.
or is a pharmaceutically acceptable salt thereof.

In another aspect, compositions, including pharmaceutical compositions and radiotracer compositions, comprising compounds described herein are provided.

In another aspect, a method of generating one or more images of a subject is provided, the method comprising administering to the subject an effective amount of a compound described herein comprising a radionuclide or a pharmaceutical composition comprising the same; and generating one or more images of at least a part of the subject's body, e.g., using positron emission tomography (PET), PET-computerized tomography (PET-CT), or single-photon emission computerized tomography (SPECT).

In another aspect of the disclosure, a method of detecting a disease in a subject is provided, the method comprising administering to the subject a compound described herein comprising a radionuclide or a pharmaceutical composition comprising the same, detecting the localization of the radionuclide in the subject using, e.g., PET, PET-CT, or SPECT, and determining the presence or absence of the disease based on the presence or absence of localization.

In another aspect of the disclosure, a method of monitoring the effect of cancer treatment on a subject afflicted with cancer is provided, the method comprising administering to the subject an effective an amount of a compound described herein comprising a radionuclide or a pharmaceutical composition comprising the same, detecting the localization of the radionuclide in the subject using, e.g., PET, PET-CT, or SPECT, and determining the effects of the cancer treatment.

In another aspect of the disclosure, a method of treating a disease in a patient afflicted with a disease is provided, the method comprising administering to the patient an effective amount of a compound or pharmaceutical composition described herein.

In another aspect of the disclosure, a theranostic method is provided, the method comprising (a) administering to a subject an effective amount of a first compound comprising a ⁶¹Cu radionuclide described herein or a pharmaceutical composition comprising the same; (b) generating one or more images of a subject (e.g., of a certain region or part of the subject's body); and (c) administering to the subject an effective amount of a compound comprising a ⁶⁷Cu radionuclide described herein or a pharmaceutical composition comprising the same, wherein the compounds in step (a) and (c) differ only by radioisotope.

### 3. BRIEF DESCRIPTION OF THE DRAWINGS

These and other features, aspects, and advantages of the present disclosure will become better understood with regard to the following description, and accompanying drawings, where:

FIG. 1 shows the partition coefficient (log D_{PBS/octanol, pH=7.4}) of ⁶¹Cu or ⁶⁸Ga-labeled conjugates. From left to right: [⁶¹Cu]Cu-NODAGA-1, [⁶¹Cu]Cu-NODAGA-3, [⁶¹Cu]Cu-NODAGA-2, [⁶¹Cu]Cu-NODAGA-4, [⁶⁸Ga]Ga-FAPI-46, and [⁶¹Cu]Cu-NODAGA-FAPI-46.

FIG. 2 shows the inhibition (IC₅₀) of ^{t}Cu-labeled conjugates.

FIG. 3, panels A-D, show cellular uptake of cell surface (cell membrane bound) and internalized fractions of [⁶¹Cu]Cu-NODAGA-1 (panel A), [⁶¹Cu]Cu-NODAGA-3 (panel B), [⁶¹Cu]Cu-NODAGA-2 (panel C), and [⁶¹Cu]Cu-NODAGA-4 (panel D). The values are expressed as % of the applied activity and refer to the specific uptake calculated after subtracting the non-specific values (measured in the presence of the non-FAP expressing cell line HT-1080.wt) from the total values (specific=total−non-specific).

FIG. 4 shows cellular uptake of cell surface (cell membrane bound) and internalized fractions of [⁶¹Cu]Cu-NODAGA-FAPI-46. The values are expressed as % of the applied activity and refer to the specific uptake calculated after subtracting the non-specific values (measured in the presence of the non-FAP expressing cell line HT-1080.wt) from the total values (specific=total−non-specific).

FIG. 5 shows the saturation binding of ⁶¹Cu-labeled conjugates on isolated HEK-293-hFAP membranes.

FIG. 6, panels A and B, show the biodistribution profiles of [⁶¹Cu]Cu-NODAGA-FAPI-46 (panel A) and [⁶⁸Ga]Ga-FAPI-46 (panel B) in HT-1080.hFAP tumor-bearing mice at 1 hour and 4 hours following administration.

FIG. 7, panels A and B, show the tumor-to-organ ratios of [⁶¹Cu]Cu-NODAGA-FAPI-46 (panel A) and [⁶⁸Ga]Ga-FAPI-46 (panel B) in HT-1080.hFAP tumor-bearing mice at 1 hour and hours following administration.

FIG. 8, panels A-B, show biodistribution profiles of [⁶¹Cu]Cu-NODAGA-1 (panel A) and [⁶¹Cu]Cu-NODAGA-3 (panel B), in HT-1080.hFAP tumor-bearing mice at 1 hour and 4 hours following administration.

FIG. 9, panels A-B, show biodistribution profiles of [⁶¹Cu]Cu-NODAGA-2 (panel A) and [⁶¹Cu]Cu-NODAGA-4 (panel B) in HT-1080.hFAP tumor-bearing mice at 1 hour and hours following administration.

FIG. 10, panels A-B, show the tumor-to-organ ratios of [⁶¹Cu]Cu-NODAGA-1 (panel A) and [⁶¹Cu]Cu-NODAGA-3 (panel B), in HT-1080.hFAP tumor-bearing mice at 1 hour and 4 hours following administration.

FIG. 11, panels A-B, show the tumor-to-organ ratios of [⁶¹Cu]Cu-NODAGA-2 (panel A), and [⁶¹Cu]Cu-NODAGA-4 (panel B) in HT-1080.hFAP tumor-bearing mice at 1 hour and 4 hours following administration.

FIG. 12 shows the dynamic PET/CT scans of [⁶¹Cu]Cu-NODAGA-1 and [⁶¹Cu]Cu-NODAGA-3 in mice bearing FAP-positive xenografts.

FIG. 13, panels A and B, show the dynamic PET/CT scans of [⁶¹Cu]Cu-NODAGA-2 (panel A) and [⁶¹Cu]Cu-NODAGA-4 (panel B) in mice bearing FAP-positive xenografts.

FIG. 14, panels A and B, show the dynamic PET/CT scans of [⁶¹Cu]Cu-NODAGA-FAPI-46 (panel A) and [⁶⁸Ga]Ga-FAPI-46 (panel B) in mice bearing FAP-positive xenografts.

FIG. 15, panels A and B, show SUV PET imaging of [⁶¹Cu]Cu-NODAGA-2 and [⁶¹Cu]Cu-NODAGA-4 (1 h and 4 h) (panel A) and [⁶¹Cu]Cu-NODAGA-FAPI-46 vs ⁶⁸Ga-FAPI-46 (1 h and 4 h for [⁶¹Cu]Cu-NODAGA-FAPI-46 and 1 h only for [⁶⁸Ga]Ga-FAPI-46) (panel B).

FIG. 16, panels A-C, display, with increasing magnification, a homogenous Ni coating having durable adhesion to the niobium coin upon completion of electroplating, as evaluated using a DINOLite digital microscope to observe the crystal structure and homogeneity of the surface.

FIG. 17 shows samples of target backing coin with nickel electrodeposited in the center of a niobium backing.

FIG. 18 shows analysis of ⁶¹Cu purity of a [⁶¹Cu]CuCl₂ solution obtained by irradiation of ^{nat}Ni on Nb backing with a deuteron beam at 8.4 MeV for 3 h at 50 μA. The line corresponds to change in % purity of ⁶¹Cu over time and the bars correspond to radiocobalt activity over time.

FIG. 19 shows analysis of ⁶¹Cu purity of a [⁶¹Cu]CuCl₂ solution obtained by irradiation of ⁶⁰Ni on Nb backing with a deuteron beam at 8.4 MeV for 3 h at 50 μA. The line corresponds to change in % purity of ⁶¹Cu over time and the bars correspond to radiocobalt activity over time.

FIG. 20 compares the radionuclidic purity of [⁶¹Cu]CuCl₂ solution that is produced with commercially available ^{nat}Ni targets on Ag backing and the radionuclidic purity of [⁶¹Cu]CuCl₂ solution produced by certain embodiments of the present disclosure when assessed by gamma spectrometry in Bq/g. Ag and Co isotopes are significantly reduced in the [⁶¹Cu]CuCl₂ solution when produced by irradiation of Ni targets electroplated according to the present disclosure on high purity Nb backing (showing specific radionuclidic impurities).

FIG. 21 displays how the radionuclidic purity of [⁶¹Cu]CuCl₂ solution that is produced with commercially available ^{nat}Ni targets (Ag backing) compares to the radionuclidic purity of [⁶¹Cu]CuCl₂ solution produced by irradiation of Ni targets electroplated according to the present disclosure on high purity Nb backing when assessed by gamma spectrometry in Bq/g (summed radionuclidic impurities). The presented data highlight in particular the reduction of overall impurities in the [⁶¹Cu]CuCl₂ solution when produced by embodiments of the present disclosure.

FIG. 22 displays how the radionuclidic purity of [⁶¹Cu]CuCl₂ solution that is produced with commercially available ^{nat}Ni targets on Ag backing compared to the radionuclidic purity of [⁶¹Cu]CuCl₂ solution produced by irradiation of Ni targets electroplated according to the present disclosure on high purity Nb backing when assessed by gamma spectrometry in Bq/g (summed radionuclidic impurities) at t=0 h and at t=12 h. The presented data highlight the superior quality of the [⁶¹Cu]CuCl₂ solution when produced by irradiation of Ni targets electroplated according to the present disclosure on high purity Nb backing, where the purity after 12 hours is still well above the purity limits set by pharmacopeia for similar radionuclides for medical use.

FIG. 23 displays chemical impurities, as measured by ICP-MS, of the [⁶¹Cu]CuCl₂ solution when produced by bombardment of ^{nat}Ni vs. ⁶¹Ni when produced by irradiation of Ni targets electroplated according to the present disclosure on high purity Nb backing.

FIG. 24 panels A-B illustrate the dynamic PET/CT scans of [⁶¹Cu]Cu-NODAGA-F1 (panel A) and [⁶¹Cu]Cu-NODAGA-F3 (panel B) in dual HT1080.hFAP and HT1080.wt tumor-bearing mice within 1 hour.

### 4. DETAILED DESCRIPTION

### 1. Definitions

When describing the embodiments of the present disclosure, which may include compounds and pharmaceutically acceptable salts thereof, pharmaceutical compositions containing such compounds and methods of using such compounds and compositions, the following terms, if present, have the following meanings unless otherwise indicated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, the term “alkyl” refers to both straight and branched chain C₁-C₃₀ hydrocarbons and includes both saturated and unsaturated hydrocarbons. The use of designations such as, for example, “C₁-C₂₀” is intended to refer to an alkyl (e.g., straight or branched chain and inclusive of alkenes and alkyls) having the recited range carbon atoms. In certain embodiments, an alkyl group has 1 to 10 carbon atoms (“C₁-C₁₀ alkyl”). In certain embodiments, an alkyl group has 1 to 9 carbon atoms (“C₁-C₉ alkyl”). In certain embodiments, an alkyl group has 1 to 8 carbon atoms (“C₁-C₈ alkyl”). In certain embodiments, an alkyl group has 1 to 7 carbon atoms (“C₁-C₇ alkyl”). In certain embodiments, an alkyl group has 1 to 6 carbon atoms (“C₁-C₆ alkyl”). In certain embodiments, an alkyl group has 1 to 5 carbon atoms (“C₁-C₅ alkyl”). In certain embodiments, an alkyl group has 1 to 4 carbon atoms (“C₁-C₄ alkyl”). In certain embodiments, an alkyl group has 1 to 3 carbon atoms (“C₁-C₃ alkyl”). In certain embodiments, an alkyl group has 1 to 2 carbon atoms (“C₁-C₂ alkyl”). In certain embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like. Representative straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like.

As used herein, the term “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂-C₂₀ alkenyl”). In certain embodiments, alkenyl does not contain any triple bonds. In certain embodiments, an alkenyl group has 2 to 10 carbon atoms (“C₂-C₁₀ alkenyl”). In certain embodiments, an alkenyl group has 2 to 9 carbon atoms (“C₂-C₉ alkenyl”). In certain embodiments, an alkenyl group has 2 to 8 carbon atoms (“C₂-C₈ alkenyl”). In certain embodiments, an alkenyl group has 2 to 7 carbon atoms (“C₂-C₇ alkenyl”). In certain embodiments, an alkenyl group has 2 to 6 carbon atoms (“C₂-C₆ alkenyl”). In certain embodiments, an alkenyl group has 2 to 5 carbon atoms (“C₂-C₅ alkenyl”). In certain embodiments, an alkenyl group has 2 to 4 carbon atoms (“C₂-C₄ alkenyl”). In certain embodiments, an alkenyl group has 2 to 3 carbon atoms (“C₂-C₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₉), and the like.

As used herein, the terms “alkylene,” “alkenylene,” and “alkynylene” refer to a divalent radical of an alkyl, alkenyl, or alkynyl group, respectively. When a range or number of carbons is provided for a particular “alkylene,” “alkenylene,” or “alkynylene,” it is understood that the range or number refers to the range or number of carbons in the linear carbon divalent chain. “Alkylene,” “alkenylene,” and “alkynylene” groups may be substituted or unsubstituted with one or more substituents as described herein.

As used herein, the term “aryl” refers to aromatic groups (e.g., monocyclic, bicyclic and tricyclic structures) containing six to ten carbons in the ring portion. The aryl groups may be optionally substituted through available carbon atoms and in certain embodiments may include one or more heteroatoms such as oxygen, nitrogen or sulfur. In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl).

As used herein, the terms “chelating moiety” or “chelator” are used interchangeably in the context of the present disclosure and refer to a molecule, often an organic molecule, and often a Lewis base, having two or more unshared electron pairs available for donation to a metal ion. The metal ion is usually coordinated by two or more electron pairs to the chelating moiety. The terms, “bidentate chelating moiety”, “tridentate chelating moiety,” and “tetradentate chelating moiety” refer to chelating moieties having, respectively, two, three, and four electron pairs readily available for simultaneous donation to a metal ion coordinated by the chelating moiety. Usually, the electron pairs of a chelating moiety form coordinate bonds with a single metal ion; however, in certain examples, a chelating moiety may form coordinate bonds with more than one metal ion, with a variety of binding modes being possible.

With respect to chemical structures that include a chelated metal, the structure as drawn is not intended to define the coordination sphere. Further, the presence or absence of a proton on an ionizable binding moiety is not intended to be definitive. A person of skill in the art will be able to determine the coordination sphere, oxidation states and degree of ionization on a case by case basis.

As used herein, the terms “effective amount,” “pharmaceutically effective amount,” or “therapeutically effective amount” mean a sufficient amount of the compound or composition to provide the desired utility when administered to a subject. The term “therapeutically effective amount” therefore refers to an amount of a compound or composition that is sufficient to promote a particular effect when administered to a subject in need of treatment. In certain embodiments, an effective amount includes an amount of compound or composition sufficient to prevent or delay the development of a symptom of the disease, alter the course of a symptom of the disease (for example but not limited to, slow the progression of a symptom of the disease), or reverse a symptom of the disease. In certain embodiments, an effective amount includes an amount of compound or composition sufficient to generate an image of subject. In certain embodiments, an effective amount includes an amount of compound or composition sufficient to diagnose a disease in a subject. It is understood that for any given case, an appropriate “effective amount” can be determined by one of ordinary skill in the art using routine experimentation. For example, when administered in clinic, such compounds or compositions will contain an amount of active ingredient effective to achieve the desired result (e.g., imaging cancerous tissue and/or decreasing an amount of cancerous tissue in a subject).

As used herein, “halo” and “halogen” refer to an atom selected from fluorine (fluoro, F), chlorine (chloro, Cl), bromine (bromo, Br), and iodine (iodo, I).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

As used herein, the term “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably. Heterocycles include morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium, and N+(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “radioactive moiety” refers to a molecular assembly which carries a radioactive nuclide. The nuclide is bound by covalent or coordinate bonds, or a combination thereof, which remain stable under physiological conditions.

As used herein, “radioisotope” refers to a radioactive isotope of an element (included by the term “radionuclide”) emitting, for example, α-, β-, and/or γ-radiation.

As used herein, “radiotracer” refers to a compound of the present disclosure comprising a radionuclide or radioisotope. The radionuclide can be chelated to a chelating moiety that is a covalently bound component of the radiotracer, or the radionuclide itself can be a covalently bound component of the radiotracer. It is understood herein that when a compound, e.g., a radiotracer, is described as comprising a particular radioisotope or radionuclide (e.g., ⁶¹Cu) that the compound is isotopically enriched in that isotope at the indicated position.

As used herein, the term “substituted”, whether preceded by the term “optionally” or not, means that at least one hydrogen present on a group (e.g., a hydrogen attached to a carbon or nitrogen atom of a group) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

In typical embodiments, the present disclosure is intended to encompass the compounds disclosed herein, and the pharmaceutically acceptable salts, pharmaceutically acceptable esters, tautomeric forms, polymorphs, and prodrugs of such compounds. In certain embodiments, the present disclosure includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

### 2. Compounds

In one aspect, the present disclosure provides FAP inhibitor compounds and compounds (also referred to a “conjugates”) comprising the novel FAP inhibitors. The FAP inhibitors and conjugates thereof can be used in the diagnosis and treatment of diseases characterized by expression of FAP.

The present disclosure provides compounds, wherein the compound is of Formula I:

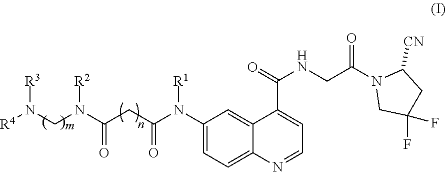

wherein:
R¹ is R^{a};
R² and R³ are each R^{a} or together form a C₂₋₉ heterocycle with the nitrogen atoms to which they are attached;
R⁴ is H, an amine protecting group, or -L-T;
R^{a}, independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl;
L is a bond or a divalent linker;
T comprises (a) a chelating moiety suitable for chelating a radionuclide (b) an imaging agent, or (c) a drug;
n is an integer from 1 to 20; and
m is an integer from 1 to 20;
   or is a pharmaceutically acceptable salt thereof.

In certain embodiments of Formula I, R¹ is H. In certain embodiments of Formula I, R¹ is selected from C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl. In certain embodiments of Formula I, R¹ is selected from H, C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula I, R¹ is selected from H and C₁₋₁₀ alkyl. In certain embodiments of Formula I, R¹ is H. In certain embodiments of Formula I, R¹ is C₁₋₁₀ alkyl. In certain embodiments of Formula I, R¹ is C₁-C₆ alkyl. In certain embodiments of Formula I, R is selected from methyl, ethyl, propyl, isopropyl, butyl, isobutyl, pentyl and hexyl. In certain embodiments of Formula I, R¹ is methyl.

In certain embodiments of Formula I, R² is H. In certain embodiments of Formula I, R² is selected from C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula I, R³ is H. In certain embodiments of Formula I, R³ is selected from C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula I, R² and R³ together form a C₂₋₉ heterocycle with the nitrogen atoms to which they are attached. In certain embodiments of Formula I, the C₂₋₉ heterocycle is a 5-, 6-, or 7-membered heterocycle. In certain embodiments of Formula I, the C₂₋₉ heterocycle is a 5-membered heterocycle selected from a pyrrolidine, pyrazolidine, and imidazoline. In certain embodiments of Formula I, the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane. In certain embodiments of Formula I, the C₂₋₉ heterocycle is a piperazine.

In certain embodiments of Formula I, R⁴ is H.

In certain embodiments of Formula I, R⁴ is an amine protecting group. In certain embodiments, the amine protecting group is selected from carbobenzyloxy (Cbz), p-methoxybenzyl carbonyl (Moz or MeOZ), tert-butyloxycarbonyl (Boc), 9-fluorenylmethyloxycarbonyl (FMOC), acetyl (Ac), benzoyl (Bz), benzyl (Bn), a carbamate, p-methoxybenzyl (PMB), 3,4-dimethoxybenzyl (DMPM), p-methoxyphenyl (PMP), a succinimide (i.e., a cyclic imide), and tosyl (Ts). In certain embodiments of Formula I, the amine protecting group is Boc.

In certain embodiments of Formula I, R⁴ is -L-T.

In certain embodiments of Formula I, n is an integer from 1 to 10. In certain embodiments of Formula I, n is an integer from 1 to 5. In certain embodiments of Formula I, n is 1, 2, 3, 4, or 5. In certain embodiments of Formula I, n is 2.

In certain embodiments of Formula I, m is an integer from 1 to 10. In certain embodiments of Formula I, m is an integer from 1 to 5. In certain embodiments of Formula I, m is 1, 2, 3, 4, or 5. In certain embodiments of Formula I, m is 2.

In certain embodiments of Formula I, R¹ is selected from H and C₁₋₁₀ alkyl, R² is H, R³ is H, R⁴ is H, n is an integer from 1 to 20, and m is an integer from 1 to 20. In certain embodiments of Formula I, R¹ is selected from H and C₁₋₁₀ alkyl, R² is H, R³ is H, R⁴ is H, n is an integer from 1 to 10, and m is an integer from 1 to 10. In certain embodiments of Formula I, R¹ is selected from H and C₁₋₁₀ alkyl, R² is H, R³ is H, R⁴ is H, n is an integer from 1 to 5, and m is an integer from 1 to 5. In certain embodiments of Formula I, R¹ is selected from H and C₁₋₁₀ alkyl, R² is H, R³ is H, R⁴ is H, n is 2, and m is 2.

In certain embodiments of Formula I, R¹ is selected from H and C₁₋₁₀ alkyl, R² is H, R³ is H, R⁴ is an amine protecting group, n is an integer from 1 to 20, and m is an integer from 1 to 20. In certain embodiments of Formula I, R¹ is selected from H and C₁₋₁₀ alkyl, R² is H, R³ is H, R⁴ is an amine protecting group, n is an integer from 1 to 10, and m is an integer from 1 to 10. In certain embodiments of Formula I, R¹ is selected from H and C₁₋₁₀ alkyl, R² is H, R³ is H, R⁴ is an amine protecting group, n is an integer from 1 to 5, and m is an integer from 1 to 5. In certain embodiments of Formula I, R¹ is selected from H and C₁₋₁₀ alkyl, R² is H, R³ is H, R⁴ is an amine protecting group, n is 2, and m is 2.

In certain embodiments of Formula I, R¹ is selected from H and C₁₋₁₀ alkyl, R² is H, R³ is H, R⁴ is -L-T, n is an integer from 1 to 20, and m is an integer from 1 to 20. In certain embodiments of Formula I, R¹ is selected from H and C₁₋₁₀ alkyl, R² is H, R³ is H, R⁴ is -L-T, n is an integer from 1 to 10, and m is an integer from 1 to 10. In certain embodiments of Formula I, R¹ is selected from H and C₁₋₁₀ alkyl, R² is H, R³ is H, R⁴ is -L-T, n is an integer from 1 to 5, and m is an integer from 1 to 5. In certain embodiments of Formula I, R¹ is selected from H and C₁₋₁₀ alkyl, R² is H, R³ is H, R⁴ is -L-T, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, R⁴ is H, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, R⁴ is H, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, R⁴ is H, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, R⁴ is H, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, R⁴ is an amine protecting group, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, R⁴ is an amine protecting group, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, R⁴ is an amine protecting group, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, R⁴ is an amine protecting group, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, R⁴ is -L-T, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, R⁴ is -L-T, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, R⁴ is -L-T, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, R⁴ is -L-T, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is H, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is H, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is H, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is H, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is an amine protecting group, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is an amine protecting group, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is an amine protecting group, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is an amine protecting group, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is -L-T, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is -L-T, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is -L-T, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, R⁴ is -L-T, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is H, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is H, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is H, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is H, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is an amine protecting group, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is an amine protecting group, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is an amine protecting group, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is an amine protecting group, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is -L-T, n is an integer from 1 to 20, and m is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is -L-T, n is an integer from 1 to 10, and m is an integer from 1 to 10. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is -L-T, n is an integer from 1 to 5, and m is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, R⁴ is -L-T, n is 2, and m is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is H, m is an integer from 1 to 20, and n is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is H, m is an integer from 1 to 10, and n is an integer from 1 to 10.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is H, m is an integer from 1 to 5, and n is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is H, m is 2, and n is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is an amine protecting group, m is an integer from 1 to 20, and n is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is an amine protecting group, m is an integer from 1 to 10, and n is an integer from 1 to 10. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is an amine protecting group, m is an integer from 1 to 5, and n is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is an amine protecting group, m is 2, and n is 2.

In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is -L-T, m is an integer from 1 to 20, and n is an integer from 1 to 20. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is -L-T, m is an integer from 1 to 10, and n is an integer from 1 to 10. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is -L-T, m is an integer from 1 to 5, and n is an integer from 1 to 5. In certain embodiments of Formula I, R¹ is selected from H and C₁₋₁₀ alkyl, R² and R³ together form a C₂₋₉ heterocycle with the nitrogen atoms to which they are attached, wherein the C₂₋₉ heterocycle is a piperazine, R⁴ is -L-T, m is 2, and n is 2.

In certain embodiments of Formula I, R² and R³ together form a piperazine with the nitrogen atoms to which they are attached and m is 2, thereby providing a compound of Formula Ia:

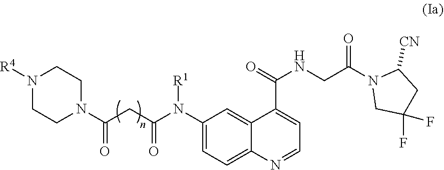

or a pharmaceutically acceptable salt thereof, wherein R¹, R⁴, and n are as described above for Formula I.

In certain embodiments of Formula Ia, R¹ is H. In certain embodiments of Formula Ia, R¹ is selected from C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula Ia, R¹ is selected from H, C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula Ia, R¹ is selected from H and C₁₋₁₀ alkyl. In certain embodiments of Formula Ia, R¹ is H. In certain embodiments of Formula Ia, R¹ is C₁₋₁₀ alkyl. In certain embodiments of Formula Ia, R¹ is C₁-C₆ alkyl. In certain embodiments of Formula Ia, R is selected from methyl, ethyl, propyl, isopropyl, butyl, isobutyl, pentyl and hexyl. In certain embodiments of Formula Ia, R¹ is methyl.

In certain embodiments of Formula Ia, R⁴ is H.

In certain embodiments of Formula Ia, R⁴ is an amine protecting group. In certain embodiments of Formula Ia, the amine protecting group is selected from carbobenzyloxy (Cbz), p-methoxybenzyl carbonyl (Moz or MeOZ), tert-butyloxycarbonyl (Boc), 9-fluorenylmethyloxycarbonyl (FMOC), acetyl (Ac), benzoyl (Bz), benzyl (Bn), a carbamate, p-methoxybenzyl (PMB), 3,4-dimethoxybenzyl (DMPM), p-methoxyphenyl (PMP), a succinimide (i.e., a cyclic imide), and tosyl (Ts). In certain embodiments of Formula Ia, the amine protecting group is Boc.

In certain embodiments of Formula Ia, R⁴ is -L-T.

In certain embodiments of Formula Ia, n is an integer from 1 to 10. In certain embodiments of Formula Ia, n is an integer from 1 to 5. In certain embodiments of Formula Ia, n is 1, 2, 3, 4, or 5. In certain embodiments of Formula Ia, n is 2.

In certain embodiments of Formula Ia, R¹ is selected from H and C₁₋₁₀ alkyl, R⁴ is H, and n is an integer from 1 to 20. In certain embodiments of Formula Ia, R¹ is selected from H and C₁₋₁₀ alkyl, R⁴ is H, and n is an integer from 1 to 10. In certain embodiments of Formula Ia, R¹ is selected from H and C₁₋₁₀ alkyl, R⁴ is H, and n is an integer from 1 to 5. In certain embodiments of Formula Ia, R¹ is selected from H and C₁₋₁₀ alkyl, R⁴ is H, and n is 2.

In certain embodiments of Formula Ia, R¹ is selected from H and C₁₋₁₀ alkyl, R⁴ is an amine protecting group, and n is an integer from 1 to 20. In certain embodiments of Formula Ia, R¹ is selected from H and C₁₋₁₀ alkyl, R⁴ is an amine protecting group, and n is an integer from 1 to 10.

In certain embodiments of Formula Ia, R¹ is selected from H and C₁₋₁₀ alkyl, R⁴ is an amine protecting group, and n is an integer from 1 to 5. In certain embodiments of Formula Ia, R¹ is selected from H and C₁₋₁₀ alkyl, R⁴ is an amine protecting group, and n is 2.

In certain embodiments of Formula Ia, R¹ is selected from H and C₁₋₁₀ alkyl, R⁴ is -L-T, and n is an integer from 1 to 20. In certain embodiments of Formula Ia, R¹ is selected from H and C₁₋₁₀ alkyl, R⁴ is -L-T, and n is an integer from 1 to 10. In certain embodiments of Formula Ia, R¹ is selected from H and C₁₋₁₀ alkyl, R⁴ is -L-T, and n is an integer from 1 to 5. In certain embodiments of Formula Ia, R¹ is selected from H and C₁₋₁₀ alkyl, R⁴ is -L-T, and n is 2.

In certain embodiments of Formula I, R² and R³ are H and m is 2, thereby providing a compound of Formula Ib:

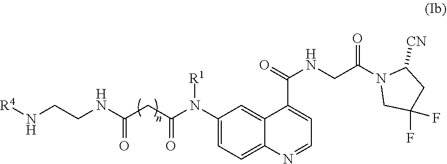

or a pharmaceutically acceptable salt thereof, wherein R¹, R⁴, and n are as described above for Formula I.

In certain embodiments of Formula Ib, R¹ is H. In certain embodiments of Formula Ib, R¹ is selected from C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula Ib, R¹ is selected from H, C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula Ib, R¹ is selected from H and C₁₋₁₀ alkyl. In certain embodiments of Formula Ib, R¹ is H. In certain embodiments of Formula Ib, R¹ is C₁₋₁₀ alkyl. In certain embodiments of Formula Ib, R¹ is C₁-C₆ alkyl. In certain embodiments of Formula Ib, R is selected from methyl, ethyl, propyl, isopropyl, butyl, isobutyl, pentyl and hexyl. In certain embodiments of Formula Ib, R¹ is methyl.

In certain embodiments of Formula Ib, R⁴ is H.

In certain embodiments of Formula Ib, R⁴ is an amine protecting group. In certain embodiments of Formula Ib, the amine protecting group is selected from carbobenzyloxy (Cbz), p-methoxybenzyl carbonyl (Moz or MeOZ), tert-butyloxycarbonyl (Boc), 9-fluorenylmethyloxycarbonyl (FMOC), acetyl (Ac), benzoyl (Bz), benzyl (Bn), a carbamate, p-methoxybenzyl (PMB), 3,4-dimethoxybenzyl (DMPM), p-methoxyphenyl (PMP), a succinimide (i.e., a cyclic imide), and tosyl (Ts). In certain embodiments of Formula Ib, the amine protecting group is Boc.

In certain embodiments of Formula Ib, R⁴ is -L-T.

In certain embodiments of Formula Ib, n is an integer from 1 to 10. In certain embodiments, n is an integer from 1 to 5. In certain embodiments, n is 1, 2, 3, 4, or 5. In certain embodiments, n is 2.

In certain embodiments of Formula Ib, R¹ is selected from H and C₁₋₁₀ alkyl, R⁴ is H, and n is an integer from 1 to 20. In certain embodiments of Formula Ib, R¹ is selected from H and C₁₋₁₀ alkyl, R⁴ is H, and n is an integer from 1 to 10. In certain embodiments of Formula Ib, R¹ is selected from H and C₁₋₁₀ alkyl, R⁴ is H, and n is an integer from 1 to 5. In certain embodiments of Formula Ib, R¹ is selected from H and C₁₋₁₀ alkyl, R⁴ is H, and n is 2.

In certain embodiments of Formula Ib, R¹ is selected from H and C₁₋₁₀ alkyl, R⁴ is an amine protecting group, and n is an integer from 1 to 20. In certain embodiments of Formula Ib, R¹ is selected from H and C₁₋₁₀ alkyl, R⁴ is an amine protecting group, and n is an integer from 1 to 10.

In certain embodiments of Formula Ib, R¹ is selected from H and C₁₋₁₀ alkyl, R⁴ is an amine protecting group, and n is an integer from 1 to 5. In certain embodiments of Formula Ib, R¹ is selected from H and C₁₋₁₀ alkyl, R⁴ is an amine protecting group, and n is 2.

In certain embodiments of Formula Ib, R¹ is selected from H and C₁₋₁₀ alkyl, R⁴ is -L-T, and n is an integer from 1 to 20. In certain embodiments of Formula Ib, R¹ is selected from H and C₁₋₁₀ alkyl, R⁴ is -L-T, and n is an integer from 1 to 10. In certain embodiments of Formula Ib, R¹ is selected from H and C₁₋₁₀ alkyl, R⁴ is -L-T, and n is an integer from 1 to 5. In certain embodiments of Formula Ib, R¹ is selected from H and C₁₋₁₀ alkyl, R⁴ is -L-T, and n is 2.

In certain embodiments, the compound of Formula I-Ib is selected from:

| Compound | Structure | |
|---|---|---|
| 1 | 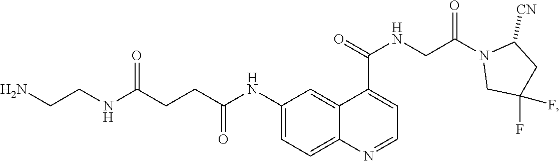 [SMILES: N#C[C@@H]1CC(F)(F)CN1C(=O)CNC(=O)c1ccnc2ccc(NC(=O)CCC(=O)NCCN)cc12] | |
| 2 | 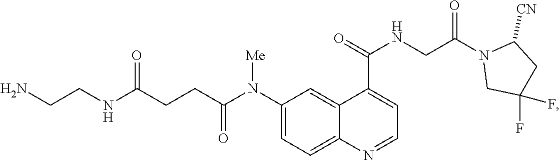 [SMILES: CN(C(=O)CCC(=O)NCCN)c1ccc2nccc(C(=O)NCC(=O)N3CC(F)(F)C[C@H]3C#N)c2c1] | |
| 3 | 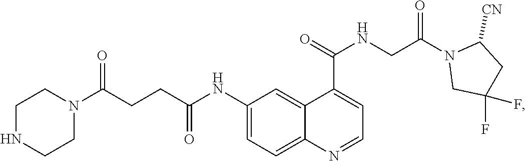 [SMILES: N#C[C@@H]1CC(F)(F)CN1C(=O)CNC(=O)c1ccnc2ccc(NC(=O)CCC(=O)N3CCNCC3)cc12] | and |

| | |
|---|---|
| 4 | 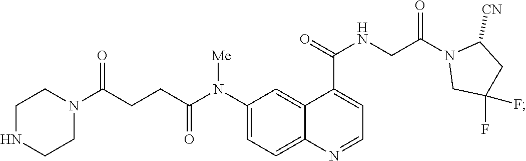 [SMILES: CN(C(=O)CCC(=O)N1CCNCC1)c1ccc2nccc(C(=O)NCC(=O)N3CC(F)(F)C[C@H]3C#N)c2c1] |

or is a pharmaceutically acceptable salt thereof.

In certain embodiments of Formula I-Ib, the pharmaceutically acceptable salt is an inorganic or organic acid salt of a compound of Formula I-Ib. In certain embodiments of Formula I-Ib, the pharmaceutically acceptable salt is an organic acid salt, e.g., the salt of an organic acid such as acetic acid, trifluoroacetic acid, oxalic acid, maleic acid, tartaric acid, citric acid, succinic acid, or malonic acid. In certain embodiments of Formula I-Ib, the pharmaceutically acceptable salt is the trifluoroacetate (TFA) salt.

In certain embodiments of Formula I-Ib, as described above, R⁴ is -L-T, wherein L is a bond or a divalent linker and T comprises (a) a chelating moiety suitable for chelating a radionuclide, (b) an imaging agent, or (c) a drug. In certain embodiments of Formula I-Ib, T is means for chelating a radionuclide.

In certain embodiments of Formula I-Ib, R⁴ is -L-T, wherein L is a bond such that the terminal nitrogen of the compound of Formula I is bound directly to T.

In certain embodiments of Formula I, R⁴ is -L-T, wherein L is a divalent linker that links, connects, or bonds to T. In certain embodiments of Formula I-Ib, L is a cleavable divalent linker. Cleavable linkers include linkers that are cleaved by intracellular metabolism following internalization, e.g., cleavage via hydrolysis, reduction, or enzymatic reaction. In certain embodiments of Formula I-Ib, L is a non-cleavable divalent linker. Non-cleavable linkers include linkers that release an attached payload via lysosomal degradation following internalization.

In certain embodiments of Formula I-Ib, L is selected from an acid-labile linker, a hydrolysis-labile linker, an enzymatically cleavable linker, a reduction labile linker, a self-immolative linker, and a non-cleavable linker.

In certain embodiments of Formula I-Ib, L comprises one or more peptides, amino acids, glucuronides, succinimide-thioethers, polyethylene glycol (PEG) units, hydrazones, mal-caproyl units, dipeptide units, valine-citruline units, para-aminobenzyl (PAB) units, or a combination thereof.

In certain embodiments of Formula I-Ib, L comprises one or more amino acids. Suitable amino acids include natural, non-natural, standard, non-standard, proteinogenic, non-proteinogenic, and L- or D-α-amino acids. In certain embodiments, the L linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, or derivatives thereof. In some embodiments, L comprises 2, 3, 4, 5, or 6 amino acids.

In certain embodiments of Formula I-Ib, R⁴ is -L-T, wherein T comprises a chelating moiety suitable for chelating a radionuclide. In certain embodiments of Formula I-Ib, the chelating moiety comprises from 2 to 8 binding moieties. In certain embodiments, of Formula I-Ib the chelating moiety is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate), NODAGA (1,4,7-triazacyclononane-1-glutaric acid-4,7-acetic acid) (also known as NOTAGA), NODA Desferoxamine (1,4,7-triazacyclononane-1,4-diyl)diacetic acid DFO), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6]eicosane-1,8-diamine), AmBaSar (4-((8-amino-3,6,10,13,16,19-hexaazabicyclo [6.6.6] icosane-1-ylamino) methyl) benzoic acid), and 4,4′-((3,6,10,13,16,19-hexaazabicyclo[6.6.6]ico-sane-1,8-diylbis(aza-nediyl))bis(methylene))dibenzoic acid (BaBaSar). In certain embodiments of Formula I-Ib, the chelating moiety is selected from DOTAGA, DOTA, NOTA, NODAGA, and NODA.

In certain embodiments of Formula I-Ib, the chelating moiety is NODAGA. In certain embodiments of Formula I-Ib, R⁴ is -L-NODAGA. In certain embodiments of Formula I-Ib, R⁴ is -L-T, wherein L is a bond, such that R⁴ is NODAGA.

In certain embodiments of Formula I-Ib, T comprises a chelating moiety that does not comprise a radionuclide.

In certain embodiments, the compound of Formula I-lb is selected from:

| Compound | Structure | |
|---|---|---|
| NODAGA-1 | 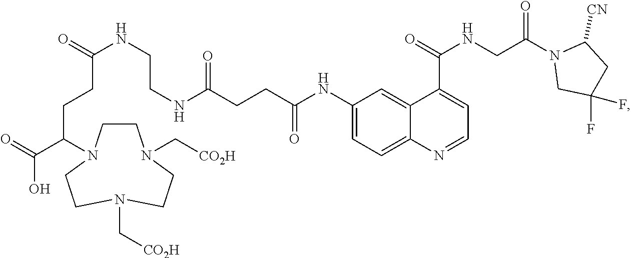 [SMILES: N#C[C@@H]1CC(F)(F)CN1C(=O)CNC(=O)c1ccnc2ccc(NC(=O)CCC(=O)NCCNC(=O)CCC(C(=O)O)N3CCN(CC(=O)O)CCN(CC(=O)O)CC3)cc12] | |
| NODAGA-2 | 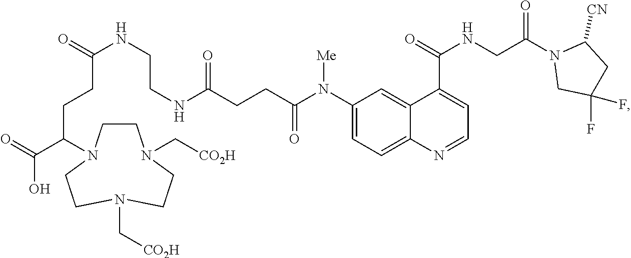 [SMILES: CN(C(=O)CCC(=O)NCCNC(=O)CCC(C(=O)O)N1CCN(CC(=O)O)CCN(CC(=O)O)CC1)c1ccc2nccc(C(=O)NCC(=O)N3CC(F)(F)C[C@H]3C#N)c2c1] | |
| NODAGA-3 | 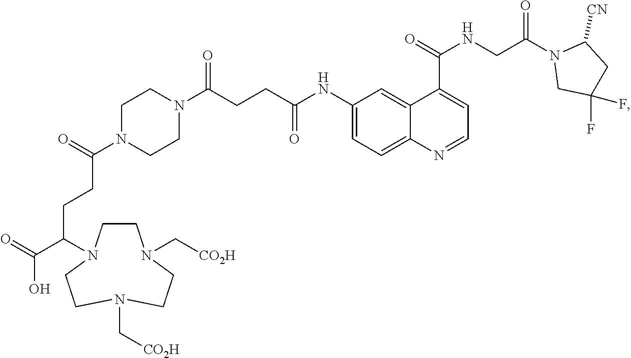 [SMILES: N#C[C@@H]1CC(F)(F)CN1C(=O)CNC(=O)c1ccnc2ccc(NC(=O)CCC(=O)N3CCN(C(=O)CCC(C(=O)O)N4CCN(CC(=O)O)CCN(CC(=O)O)CC4)CC3)cc12] | and |

| | |
|---|---|
| NODAGA-4 | 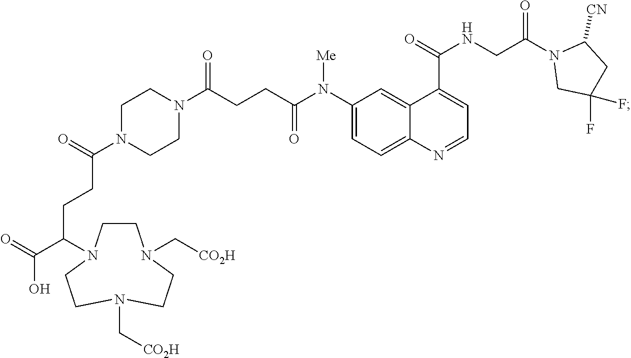 [SMILES: CN(C(=O)CCC(=O)N1CCN(C(=O)CCC(C(=O)O)N2CCN(CC(=O)O)CCN(CC(=O)O)CC2)CC1)c1ccc2nccc(C(=O)NCC(=O)N3CC(F)(F)C[C@H]3C#N)c2c1] |

or is a pharmaceutically acceptable salt thereof.

In certain embodiments of Formula I-Ib, T comprises a chelating moiety chelated to a radionuclide. In certain embodiments of Formula I-Ib, the radionuclide is selected from alpha radiation emitting isotopes, beta radiation emitting isotopes, gamma radiation emitting isotopes, Auger electron emitting isotopes, X-ray emitting isotopes, and fluorescence emitting isotopes. In certain embodiments of Formula I-Ib, the radionuclide is selected from ²²⁵Ac, ⁵¹Cr, ⁶⁶Ga, ⁶⁷Ga, ⁶⁸GA, [¹⁸F]AlF, ¹¹¹In, ^{113m}In, ^{52m}Mn, ^{99m}Tc, ¹⁸⁶Re, ¹⁸⁸Re, ¹³⁹La, ¹⁴⁰La, ¹⁷⁵Yb, ¹⁷⁹Yb, ¹⁵³Sm, ^{177m}Sn, ¹⁶⁶Ho, ⁸⁶Y, ⁸⁸Y, ⁹⁰Y, ¹⁴⁹Pm, ¹⁶⁵Dy, ¹⁶⁹Er, ¹⁷⁷Lu, ⁵²Fe, ⁴³Sc, ⁴⁴Sc, ⁴⁶Sc, ⁴⁷Sc, ¹⁴²Pr, ¹⁵⁷Gd, ¹⁵⁹Gd, ²¹²Bi, ²¹³Bi, ⁷²As, ⁷⁷As, ⁹⁷Ru, ¹⁰⁹Pd, ¹⁰⁵Rh, ^{101m}Rh, ¹¹⁹Sb, ¹⁹⁷Hg, ¹⁵¹Eu, ¹⁵³Eu, ¹⁶⁹Eu, ²⁰¹Tl, ¹⁴⁹Tb, ¹⁵²Tb, ¹⁵⁵Tb, ¹⁶¹Tb, ²⁰³Pb, ²¹²Pb, ¹⁵¹Pm, ¹⁵³Pm, ¹⁴²Pr, ¹⁴³Pr, ⁵⁵Co, ⁶⁰Cu, ⁶¹Cu, ⁶²Cu, ⁶⁴Cu, ⁶⁷Cu, ⁶²Zn, ¹⁸⁸Re, ¹⁹⁸Au, ¹⁹⁹Au, ²²⁷Th, ¹¹¹Ag, ¹⁹⁹Ag, ²¹¹At, ²²³Ra, ⁸⁸Zr, and ⁸⁹Zr.

In certain embodiments of Formula I-Ib, the radionuclide is selected from ⁶¹Cu, ⁶²Cu, ⁶⁴Cu, and ⁶⁷Cu. In certain embodiments of Formula I-Ib, the radionuclide is ⁶¹Cu. In certain embodiments of Formula I-Ib, the radionuclide is ⁶⁷Cu.

In certain embodiments of Formula I-Ib, R¹ is -L-NODAGA-*Cu, wherein *Cu is selected from ⁶¹Cu, ⁶²Cu, ⁶⁴Cu, and ⁶⁷Cu. In certain embodiments of Formula I-Ib, R is -L-T, wherein L is a bond, such that R is NODAGA-*Cu, wherein *Cu is selected frorn ⁶¹Cu, ⁶²Cu, ⁶⁴Cu, and ⁶⁷Cu.

In certain embodiments of Formula I-Ib, R¹ is selected from H and methyl; R⁴ is -L-T, wherein T comprises a chelating moiety chelated to a radionuclide, wherein the radionuclide is selected from ⁶¹Cu, ⁶⁴Cu, and ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the radionuclide is ⁶¹Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the radionuclide is ⁶⁴Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the radionuclide is ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is selected from DOTAGA, DOTA, NOTA, NODAGA, and NODA; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTAGA; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTA; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NOTA; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODAGA; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODA; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is selected from DOTAGA, DOTA, NOTA, NODAGA, and NODA, wherein the radionuclide is selected from ⁶¹Cu, ⁶⁴Cu, and ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTAGA and the radionuclide is selected from ⁶¹Cu, ⁶⁴Cu, and ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTAGA and the radionuclide is ⁶¹Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTAGA and the radionuclide is ⁶⁴Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTAGA and the radionuclide is ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTA and the radionuclide is selected from ⁶¹Cu, ⁶⁴Cu, and ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTA and the radionuclide is ⁶¹Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTA and the radionuclide is ⁶⁴Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is DOTA and the radionuclide is ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NOTA and the radionuclide is selected from ⁶¹Cu, ⁶⁴Cu, and ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NOTA and the radionuclide is ⁶¹Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NOTA and the radionuclide is ⁶⁴Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NOTA and the radionuclide is ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODAGA and the radionuclide is selected from ⁶¹Cu, ⁶⁴Cu, and ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODAGA and the radionuclide is ⁶¹Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODAGA and the radionuclide is ⁶⁴Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODAGA and the radionuclide is ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODA and the radionuclide is selected from ⁶¹Cu, ⁶⁴Cu, and ⁶⁷Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODA and the radionuclide is ⁶¹Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODA and the radionuclide is ⁶¹Cu; and n is an integer from 1 to 5.

In certain embodiments of Formula I-Ib, R¹ is selected from H and methyl; R⁴ is -L-T, wherein T comprises a chelating moiety chelated to a radionuclide, wherein the chelating moiety is NODA and the radionuclide is ⁶⁷Cu; and n is an integer from 1 to 5.

In certain embodiments, the compound of Formula I-Ib is selected from:

| Compound | Structure | |
|---|---|---|
| *Cu-NODAGA-1 | 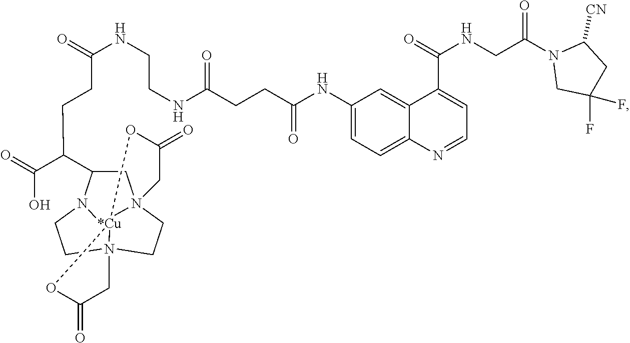 | |
| *Cu-NODAGA-2 | 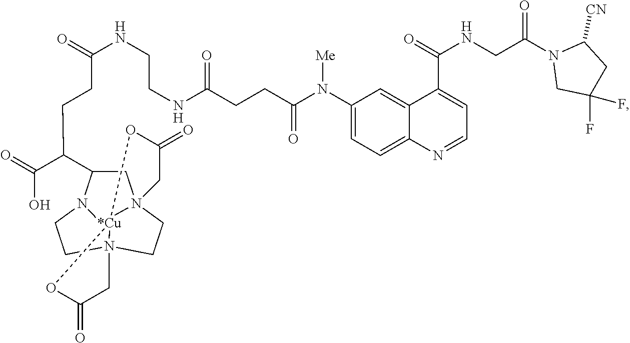 | |
| *Cu-NODAGA-3 | 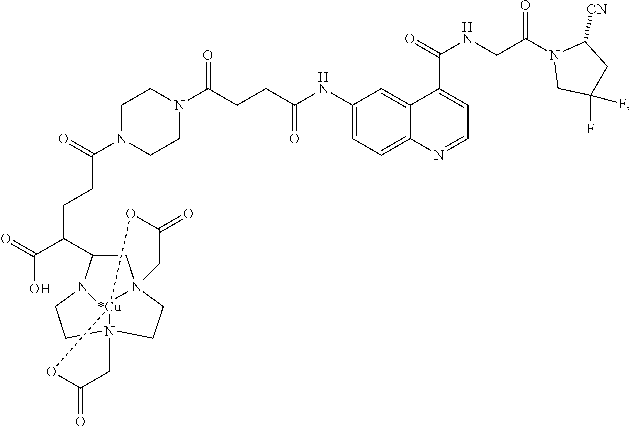 | and |

| | |
|---|---|
| *Cu-NODAGA-4 | 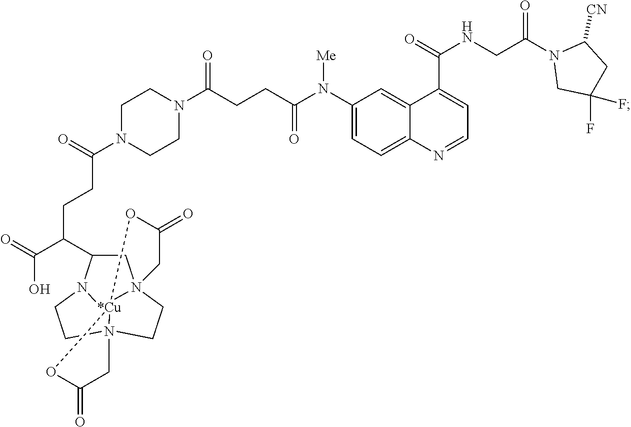 |

or is a pharmaceutically acceptable salt thereof, wherein *Cu is selected from ⁶¹Cu, ⁶²Cu, ⁶⁴Cu, and ⁶⁷Cu, particularly from ⁶¹Cu and ⁶⁷Cu.

In certain embodiments of Formula I-Ib, T comprises an imaging agent. In certain embodiments of Formula I-Ib, the imaging agent comprises a radionuclide. In certain embodiments of Formula I-Ib, the radionuclide is selected from ¹⁸F, ¹⁴C, ¹¹C, ¹³N, ³²P, ³⁵S, ¹²⁵I, ¹³¹I, ¹²⁴I, ¹²³I, and ¹⁵O.

In certain embodiments of Formula I-Ib, the imaging agent comprises a non-chelating radioactive moiety. In certain embodiments of Formula I-Ib, the non-chelating radioactive moiety is selected from [¹¹C]Cu-methionine (Met), [¹⁸F]F-2-fluoro-2-deoxyglucose (FDG), [¹⁸F]F-labeled C₆₋₁₀ aryl, and [¹⁸F]F-labeled C₅₋₉ heteroaryl.

In certain embodiments of Formula I-Ib, the imaging agent comprises a chelating moiety chelated to a radionuclide. In certain embodiments of Formula I-Ib, the chelating moiety comprises from 2 to 8 binding moieties. In certain embodiments of Formula I-Ib, the chelating moiety is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate), NODAGA (1,4,7-triazacyclononane-1-glutaric acid-4,7-acetic acid) (also known as NOTAGA), NODA Desferoxamine (1,4,7-triazacyclononane-1,4-diyl)diacetic acid DFO), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), and 4,4′-((3,6,10,13,16,19-hexaazabicyclo[6.6.6]ico-sane-1,8-diylbis(aza-nediyl))bis(methylene))dibenzoic acid (BaBaSar). In certain embodiments of Formula I-Ib, the chelating moiety is selected from DOTAGA, DOTA, NOTA, NODAGA, and NODA. In some embodiments of Formula I-Ib, the chelating moiety is NODAGA.

In certain embodiments of Formula I-Ib, the radionuclide chelated to the chelating moiety of the imaging agent is selected from alpha radiation emitting isotopes, beta radiation emitting isotopes, gamma radiation emitting isotopes, Auger electron emitting isotopes, X-ray emitting isotopes, and fluorescence emitting isotopes. In certain embodiments of Formula I-Ib, the radionuclide is selected from ²²⁵Ac, ⁵¹Cr, ⁶⁶Ga, ⁶⁷Ga, ⁶⁸GA, [¹⁸F]AlF, ¹¹¹In, ^{113m}In, ^{52m}Mn, ^{99m}Tc, ¹⁸⁶Re, ¹⁸⁸Re, ¹³⁹La, ¹⁴⁰La, ¹⁷⁵Yb, ¹⁷⁹Yb, ¹⁵³Sm, ^{177m}Sn, ¹⁶⁶Ho, ⁸⁶Y, ⁸⁸Y, ⁹⁰Y, ¹⁴⁹Pm, ¹⁶⁵Dy, ¹⁶⁹Er, ¹⁷⁷Lu, ⁵²Fe, ⁴³Sc, ⁴⁴Sc, ⁴⁶Sc, ⁴⁷Sc, ¹⁴²Pr, ¹⁵⁷Gd, ¹⁵⁹Gd, ²¹²Bi, ²¹³Bi, ⁷²As, ⁷⁷As, ⁹⁷Ru, ¹⁰⁹Pd, ¹⁰⁵Rh, ^{101m}Rh, ¹¹⁹Sb, ¹⁹⁷Hg, ¹⁵¹Eu, ¹⁵³Eu, ¹⁶⁹Eu, ²⁰¹Tl, ¹⁴⁹Tb, ¹⁵²Tb, ¹⁵⁵Tb, ¹⁶¹Tb, ²⁰³Pb, ²¹²Pb, ¹⁵¹Pm, ¹⁵³Pm, ¹⁴²Pr, ¹⁴³Pr, ⁵⁵Co, ⁶⁰Cu, ⁶¹Cu, ⁶²Cu, ⁶⁴Cu, ⁶⁷Cu, ⁶²Zn, ¹⁸⁸Re, ¹⁹⁸Au, ¹⁹⁹Au, ²²⁷Th, ¹¹¹Ag, ¹⁹⁹Ag, ²¹¹At, ²²³Ra, ⁸⁸Zr, and ⁸⁹Zr. In certain embodiments of Formula I-Ib, the radionuclide chelated within the chelating moiety of the imaging agent is ⁶¹Cu.

In certain embodiments of Formula I-Ib, T comprises an imaging agent comprising a chelating moiety chelated to a radionuclide. In certain embodiments of Formula I-Ib, T comprises ⁶¹Cu chelated to NODAGA (⁶¹Cu-NODAGA). In certain embodiments of Formula I-Ib, T is ⁶¹ chelated to NODAGA (⁶¹Cu-NODAGA).

In certain embodiments of Formula I-Ib, the imaging agent is a fluorescent dye. In certain embodiments, the fluorescent dye is selected from one of the following classes: xanthens, acridines, oxazines, cynines, styryl dyes, coumarines, porphines, metal-ligand-complexes, fluorescent proteins, nanocrystals, perylenes, boron-dipyrromethenes, and phtalocyanines.

In certain embodiments of Formula I-Ib, T comprises a drug.

In certain embodiments of Formula I-Ib, the drug comprises a chelating moiety chelated to a radionuclide. In certain embodiments, the chelating moiety comprises from 2 to 8 binding moieties. In certain embodiments, the chelating moiety is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate), NODAGA (1,4,7-triazacyclononane-1-glutaric acid-4,7-acetic acid) (also known as NOTAGA), NODA Desferoxamine (1,4,7-triazacyclononane-1,4-diyl)diacetic acid DFO), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), and 4,4′-((3,6,10,13,16,19-hexaazabicyclo[6.6.6]ico-sane-1,8-diylbis(aza-nediyl))bis(methylene))dibenzoic acid (BaBaSar). In certain embodiments, the chelating moiety is selected from DOTAGA, DOTA, NOTA, NODAGA, and NODA. In certain embodiments, the chelating moiety is NODAGA.

In certain embodiments of Formula I-Ib, the radionuclide chelated to the chelating moiety of the drug is selected from alpha radiation emitting isotopes and beta radiation emitting isotopes. In certain embodiments of Formula I-Ib, the radionuclide chelated to the chelating moiety of the drug is an alpha radiation emitting isotope. In certain embodiments of formula I-Ib, the radionuclide chelated to the chelating moiety of the drug is selected from ²²⁵Ac, ⁵¹Cr, ⁶⁶Ga, ⁶⁷Ga, ⁶⁸GA, [¹⁸F]AlF, ¹¹¹In, ^{113m}In, ^{52m}Mn, ^{99m}Tc, ¹⁸⁶Re, ¹⁸⁸Re, ¹³⁹La, ¹⁴⁰La, ¹⁷⁵Yb, ¹⁷⁹Yb, ¹⁵³Sm, ^{177m}Sn, ¹⁶⁶Ho, ⁸⁶Y, ⁸⁸Y, ⁹⁰Y, ¹⁴⁹Pm, ¹⁶⁵Dy, ¹⁶⁹Er, ¹⁷⁷Lu, ⁵²Fe, ⁴³Sc, ⁴⁴Sc, ⁴⁶Sc, ⁴⁷Sc, ¹⁴²Pr, ¹⁵⁷Gd, ¹⁵⁹Gd, ²¹²Bi, ²¹³Bi, ⁷²As, ⁷⁷As, ⁹⁷Ru, ¹⁰⁹Pd, ¹⁰⁵Rh, ^{101m}Rh, ¹¹⁹Sb, ¹⁹⁷Hg, ¹⁵¹Eu, ¹⁵³Eu, ¹⁶⁹Eu, ²⁰¹Tl, ¹⁴⁹Tb, ¹⁵²Tb, ¹⁵⁵Tb, ¹⁶¹Tb, ²⁰³Pb, ²¹²Pb, ¹⁵¹Pm, ¹⁵³Pm, ¹⁴²Pr, ¹⁴³Pr, ⁵⁵Co, ⁶⁰Cu, ⁶¹Cu, ⁶²Cu, ⁶⁴Cu, ⁶⁷Cu, ⁶²Zn, ¹⁸⁸Re, ¹⁹⁸Au, ¹⁹⁹Au, ²²⁷Th, ¹¹¹Ag, ¹⁹⁹Ag, ²¹¹At, ²²³Ra, ⁸⁸Zr, and ⁸⁹Zr. In certain embodiments of Formula I-Ib, the radionuclide chelated to the chelating moiety of the drug is ⁶⁷Cu.

In certain embodiments of Formula I-Ib, T comprises a drug comprising a chelating moiety chelated to a radionuclide. In certain embodiments of Formula I-Ib, T comprises ⁶⁷Cu chelated to NODAGA ([⁶⁷Cu]Cu-NODAGA). In certain embodiments of Formula I-Ib, T is ⁶⁷Cu chelated to NODAGA ([⁶⁷Cu]Cu-NODAGA).

In certain embodiments of Formula I-Ib, the drug is a cytotoxic agent. In certain embodiments of Formula I-Ib, cytotoxic agent selected from adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, methotrexate, busulfan, carboplatin, chlorambucil, cisplatin and other platinum compounds, taxanes, such as tamoxiphen, taxol, paclitaxel, paclitaxel derivatives, Taxoteret®, and the like, maytansines and analogs and derivatives thereof, cyclophosphamide, daunomycin, doxorubicin, rhizoxin, T2 toxin, plant alkaloids, prednisone, hydroxyurea, teniposide, mitomycins, discodermolides, microtubule inhibitors, epothilones, tubulysin, cyclopropyl benz[e]indolone, seco-cyclopropyl benz[e]indolone, O—Ac-seco-cyclopropyl benz[e]indolone, bleomycin and any other antibiotic, nitrogen mustards, nitrosureas, vincristine, vinblastine, and analogs and derivative thereof such as deacetylvinblastine monohydrazide, colchicine, colchicine derivatives, allocolchicine, thiocolchicine, trityl cysteine, Halicondrin B, dolastatins such as dolastatin 10, amanitins such as α-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, penicillins, cephalosporins, vancomycin, erythromycin, clindamycin, rifampin, chloramphenicol, aminoglycoside antibiotics, gentamicin, amphotericin B, acyclovir, trifluridine, ganciclovir, zidovudine, amantadine, and ribavirin.

In certain embodiments of Formula I-Ib, the drug is selected from peptides, oligopeptides, retro-inverso oligopeptides, proteins, protein analogs in which at least one non-peptide linkage replaces a peptide linkage, apoproteins, glycoproteins, enzymes, coenzymes, enzyme inhibitors, amino acids and their derivatives, receptors and other membrane proteins; antigens and antibodies thereto; haptens and antibodies thereto; hormones, lipids, phospholipids, liposomes; toxins; antibiotics; analgesics; bronchodilators; beta-blockers; antimicrobial agents; antihypertensive agents; cardiovascular agents including antiarrhythmics, cardiac glycosides, antianginals and vasodilators; central nervous system agents including stimulants, psychotropics, antimanics, and depressants; antiviral agents; antihistamines; cancer drugs including chemotherapeutic agents; tranquilizers; anti-depressants; H-2 antagonists; anticonvulsants; antinauseants; prostaglandins and prostaglandin analogs; muscle relaxants; anti-inflammatory substances; stimulants; decongestants; antiemetics; diuretics; antispasmodics; antiasthmatics; anti-Parkinson agents; expectorants; cough suppressants; mucolytics; and mineral and nutritional additives.

The present disclosure also provides further compounds, including when the compound is a compound of Formula II:

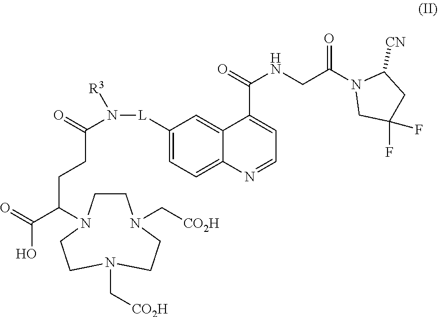

wherein:
R³ is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; or
R³, together with a moiety in L, form a C₂₋₉ heterocycle with the nitrogen atom(s) to which they are attached; and
L is a divalent linker, preferably up to 20 atoms in length;
   or is a pharmaceutically acceptable salt thereof.

In certain embodiments of Formula II, R³ is H. In certain embodiments of Formula II, R³ is C₁-C₁₀ alkyl. In certain embodiments of Formula II, R³ is C₁-C₆ alkyl. In certain embodiments of Formula II, R³ is selected from methyl, ethyl, propyl, isopropyl, butyl, isobutyl, pentyl and hexyl. In certain embodiments of Formula II, R³ is methyl.

In certain embodiments of Formula II, L is a divalent linker as described above for Formula I.

In certain embodiments of Formula II, L is up to 20 atoms in length (meaning up to 20 atoms are connected sequentially to form the backbone of L extending from each of L's two bonding sites to the rest of the compound). In certain embodiments of Formula II, L is up to 15 atoms in length. In certain embodiments of Formula II, L is up to 10 atoms in length. In certain embodiments of Formula II, L is up to 5 atoms in length.

In certain embodiments of Formula II, L comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula II, L comprises one or more —N(R²)— groups, wherein R², independently for each occurrence, is H or C₁₋₁₀ alkyl, e.g., methyl.

In certain embodiments of Formula II, L comprises one or more —C(═O)— groups.

In certain embodiments of Formula II, L comprises one or more C₁₋₁₀ alkylene groups.

In certain embodiments of Formula II, L comprises one or more —N(R²)— groups and one or more —C(═O)— groups. In certain embodiments of Formula II, L comprises one or more —N(R²-groups and one or more C₁₋₁₀ alkylene groups. In certain embodiments of Formula II, L comprises one or more —C(═O)— groups and one or more C₁₋₁₀ alkylene groups. In certain embodiments of Formula II, L comprises one or more —N(R²)— groups, one or more —C(═O)— groups, and one or more C₁₋₁₀ alkylene groups.

In certain embodiments of Formula II, R³, together with a moiety in L, form a C₂₋₉ heterocycle with the nitrogen atom(s) to which they are attached. In certain embodiments of Formula II, the C₂₋₉ heterocycle is a 5-, 6-, or 7-membered heterocycle. In certain embodiments of Formula II, the C₂₋₉ heterocycle is a 5-membered heterocycle selected from a pyrrolidine, pyrazolidine, and imidazoline. In certain embodiments of Formula II, the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane. In certain embodiments of Formula II, the C₂₋₉ heterocycle is a piperazine.

In certain embodiments of Formula II, R³ is H, L is up to 20 atoms in length and comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl. In certain embodiments of Formula II, R³ is H and L is up to 20 atoms in length and comprises one or more occurrences of groups selected from —N(R²)—, —O—, —C(═O)—, C₁₋₁₀ alkylene, and combinations thereof, wherein R², independently and or each occurrence, is selected from H and C₁₋₁₀ alkyl.

In certain embodiments of Formula II, R³ is H and L is up to 20 atoms in length and comprises one or more —N(R²)— groups, wherein R², independently for each occurrence, is H or C₁₋₁₀ alkyl, preferably methyl.

In certain embodiments of Formula II, R³ is H and L is up to 20 atoms in length and comprises one or more —C(═O)— groups.

In certain embodiments of Formula II, R³ is H and L is up to 20 atoms in length and comprises one or more C₁₋₁₀ alkylene groups.

In certain embodiments of Formula II, R³ is H and L is up to 20 atoms in length and comprises one or more —C(═O)— groups and one or more or more C₁₋₁₀ alkylene groups. In certain embodiments of Formula II, R³ is H and L is up to 20 atoms in length and comprises one or more —C(═O)— groups and one or more —N(R²)— groups, wherein R², independently for each occurrence, is H or C₁₋₁₀ alkyl, preferably methyl. In certain embodiments of Formula II, R³ is H and L is up to 20 atoms in length and comprises one or more C₁₋₁₀ alkylene groups and one or more —N(R²)— groups, wherein R², independently for each occurrence, is H or C₁₋₁₀ alkyl, preferably methyl. In certain embodiments of Formula II, R³ is H and L is up to 20 atoms in length and comprises one or more —C(═O)— groups, one or more C₁₋₁₀ alkylene groups, and one or more —N(R²)— groups, wherein R², independently for each occurrence, is H or C₁₋₁₀ alkyl, preferably methyl.

In certain embodiments of Formula II, R³, together with a moiety in L, form a C₂₋₉ heterocycle with the nitrogen atom(s) to which they are attached, wherein L is up to 20 atoms in length and comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula II, R³, together with a moiety in L, form a C₂₋₉ heterocycle with the nitrogen atom(s) to which they are attached, wherein L is up to 20 atoms in length and comprises one or more occurrences of groups selected from —N(R²)—, —O—, —C(═O)—, C₁₋₁₀ alkylene, and combinations thereof, wherein R², independently for each occurrence, is selected from H and C₁₋₁₀ alkyl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula II, R³, together with a moiety in L, form a C₂₋₉ heterocycle with the nitrogen atom(s) to which they are attached, wherein L is up to 20 atoms in length and comprises one or more occurrences of —N(R²)—, wherein R², independently for each occurrence, is H or C₁₋₁₀ alkyl.

In certain embodiments of Formula II, the compound is a compound of Formula IIa:

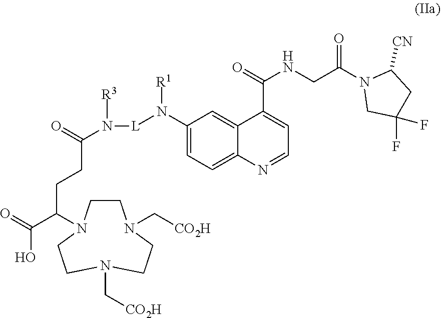

wherein:
R¹ is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; and
R³ and L are as described above for Formula II;
   or is a pharmaceutically acceptable salt thereof.

In certain embodiments of Formula IIa, R¹ is H. In certain embodiments of Formula IIa, R¹ is C₁-C₁₀ alkyl. In certain embodiments of Formula IIa, R¹ is C₁-C₆ alkyl. In certain embodiments of Formula IIa, R¹ is selected from methyl, ethyl, propyl, isopropyl, butyl, isobutyl, pentyl and hexyl. In certain embodiments of Formula IIa, R¹ is methyl.

In certain embodiments of Formula IIa, R³ is H. In certain embodiments of Formula IIa, R³ is C₁-C₁₀ alkyl. In certain embodiments of Formula IIa, R³ is C₁-C₆ alkyl. In certain embodiments of Formula IIa, R³ is selected from methyl, ethyl, propyl, isopropyl, butyl, isobutyl, pentyl and hexyl. In certain embodiments of Formula IIa, R³ is methyl.

In certain embodiments of Formula IIa, R³, together with a moiety in L, form a C₂₋₉ heterocycle with the nitrogen atom(s) to which they are attached. In certain embodiments of Formula IIa, the C₂₋₉ heterocycle is a 5-, 6-, or 7-membered heterocycle. In certain embodiments of Formula IIa, the C₂₋₉ heterocycle is a 5-membered heterocycle selected from a pyrrolidine, pyrazolidine, and imidazoline. In certain embodiments of Formula IIa, the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane. In certain embodiments of Formula IIa, the C₂₋₉ heterocycle is a piperazine.

In certain embodiments of Formula IIa, L is a divalent linker as described above for Formula I.

In certain embodiments of Formula IIa, L is up to 20 atoms in length. In certain embodiments of Formula IIa, L is up to 15 atoms in length. In certain embodiments of Formula IIa, L is up to 10 atoms in length. In certain embodiments of Formula IIa, L is up to 5 atoms in length.

In certain embodiments of Formula IIa, L comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula IIa, L comprises one or more —N(R²)—, wherein R², independently for each occurrence, is H or C₁₋₁₀ alkyl, e.g., methyl.

In certain embodiments of Formula IIa, L comprises one or more —C(═O)— groups.

In certain embodiments of Formula IIa, L comprises one or more C₁₋₁₀ alkylene groups.

In certain embodiments of Formula IIa, L comprises one or more —N(R²)— groups and one or more —C(═O)— groups. In certain embodiments of Formula IIa, L comprises one or more —N(R²-groups and one or more C₁₋₁₀ alkylene groups. In certain embodiments of Formula IIa, L comprises one or more —C(═O)— groups and one or more C₁₋₁₀ alkylene groups. In certain embodiments of Formula IIa, L comprises one or more —N(R²)— groups, one or more —C(═O)— groups, and one or more C₁₋₁₀ alkylene groups.

In certain embodiments of Formula IIa, R³, together with a moiety in L, form a C₂₋₉ heterocycle with the nitrogen atom(s) to which they are attached. In certain embodiments of Formula IIa, the C₂₋₉ heterocycle is a 5-, 6-, or 7-membered heterocycle. In certain embodiments of Formula IIa, the C₂₋₉ heterocycle is a 5-membered heterocycle selected from a pyrrolidine, pyrazolidine, and imidazoline. In certain embodiments of Formula IIa, the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane. In certain embodiments of Formula IIa, the C₂₋₉ heterocycle is a piperazine.

In certain embodiments of Formula IIa, R¹ is H or C₁₋₁₀ alkyl; R³ is H; and L is up to 20 atoms in length and comprises one or more occurrences of groups selected from —N(R²)—, —O—, —S—, —C(═NR₂)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl. In certain embodiments of Formula IIa, R¹ is H or C₁₋₁₀ alkyl; R³ is H; and L is up to 20 atoms in length and comprises one or more occurrences of groups selected from —N(R²)—, —O—, —C(═O)—, C₁₋₁₀ alkylene, and combinations thereof, wherein R², independently and or each occurrence, is selected from H and C₁₋₁₀ alkyl.

In certain embodiments of Formula IIa, R¹ is H or C₁₋₁₀ alkyl; R³ is H; and L is up to 20 atoms in length and comprises one or more —N(R²)— groups, wherein R², independently for each occurrence, is H or C₁₋₁₀ alkyl.

In certain embodiments of Formula IIa, R¹ is H or C₁₋₁₀ alkyl; R³ is H; and L is up to 20 atoms in length and comprises one or more —C(═O)— groups.

In certain embodiments of Formula IIa, R¹ is H or C₁₋₁₀ alkyl; R³ is H; and L is up to 20 atoms in length and comprises one or more or more C₁₋₁₀ alkylene groups.

In certain embodiments of Formula IIa, R¹ is H or C₁₋₁₀ alkyl; R³ is H; and L is up to 20 atoms in length and comprises one or more —C(═O)— groups and one or more C₁₋₁₀ alkylene groups.

In certain embodiments of Formula IIa, R¹ is H or C₁₋₁₀ alkyl; R³ is H; and L is up to 20 atoms in length and comprises one or more —C(═O)— groups and one or more —N(R²)— groups, wherein R², independently for each occurrence, is H or C₁₋₁₀ alkyl. In certain embodiments of Formula IIa, R¹ is H or C₁₋₁₀ alkyl; R³ is H; and L is up to 20 atoms in length and comprises one or more C₁₋₁₀ alkylene groups and one or more —N(R²)— groups, wherein R², independently for each occurrence, is H or C₁₋₁₀ alkyl. In certain embodiments of Formula IIa, R¹ is H or C₁₋₁₀ alkyl; R³ is H; and L is up to 20 atoms in length and comprises one or more —C(═O)— groups, one or more C₁₋₁₀ alkylene groups, and one or more —N(R²)— groups, wherein R², independently for each occurrence, is H or C₁₋₁₀ alkyl.

In certain embodiments of Formula IIa, R¹ is H or C₁₋₁₀ alkyl; and R³, together with a moiety in L, form a C₂₋₉ heterocycle with the nitrogen atom(s) to which they are attached; wherein L is up to 20 atoms in length and comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula IIa, R¹ is H or C₁₋₁₀ alkyl; R³, together with a moiety in L, form a C₂₋₉ heterocycle with the nitrogen atom(s) to which they are attached; wherein L is up to 20 atoms in length and comprises one or more occurrences of groups selected from —N(R²)—, —O—, —C(═O)—, C₁₋₁₀ alkylene, and combinations thereof, wherein R², independently for each occurrence, is selected from H and C₁₋₁₀ alkyl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula IIa, R¹ is H or C₁₋₁₀ alkyl; R³, together with a moiety in L, form a C₂₋₉ heterocycle with the nitrogen atom(s) to which they are attached; wherein L is up to 20 atoms in length and comprises one or more occurrences of-N(R²)—, wherein R², independently for each occurrence, is H or C₁₋₁₀ alkyl.

The present disclosure also provides yet further compounds, including when the compound is a compound of Formula III:

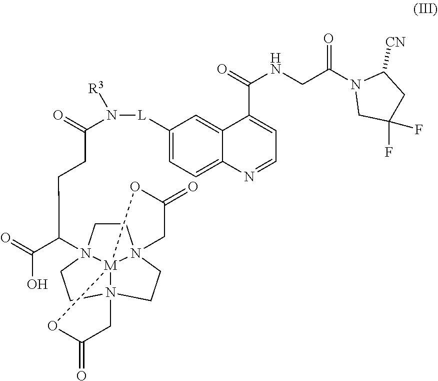

wherein:
R³ is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; or
R³, together with a moiety in L, form a C₂₋₉ heterocycle with the nitrogen atom(s) to which they are attached;
L is a divalent linker, preferably up to 20 atoms in length; and
M is selected from ²²⁵Ac, ⁵¹Cr, ⁶⁶Ga, ⁶⁷Ga, ⁶⁸GA, [¹⁸F]AlF, ¹¹¹In, ^{113m}In, ^{52m}Mn, ^{99m}Tc, ¹⁸⁶Re, ¹⁸⁸Re, ¹³⁹La, ¹⁴⁰La, ¹⁷⁵Yb, ¹⁷⁹Yb, ¹⁵³Sm, ^{177m}Sn, ¹⁶⁶Ho, ⁸⁶Y, ⁸⁸Y, ⁹⁰Y, ¹⁴⁹Pm, ¹⁶⁵Dy, ¹⁶⁹Er, ¹⁷⁷Lu, ⁵²Fe, ⁴³Sc, ⁴⁴Sc, ⁴⁶Sc, ⁴⁷Sc, ¹⁴²Pr, ¹⁵⁷Gd, ¹⁵⁹Gd, ²¹²Bi, ²¹³Bi, ⁷²As, ⁷⁷As, ⁹⁷Ru, ¹⁰⁹Pd, ¹⁰⁵Rh, ^{101m}Rh, ¹¹⁹Sb, ¹⁹⁷Hg, ¹⁵¹Eu, ¹⁵³Eu, ¹⁶⁹Eu, ²⁰¹Tl, ¹⁴⁹Tb, ¹⁵²Tb, ¹⁵⁵Tb, ¹⁶¹Tb, ²⁰³Pb, ²¹²Pb, ¹⁵¹Pm, ¹⁵³Pm, ¹⁴²Pr, ¹⁴³Pr, ⁵⁵Co, ⁶⁰Cu, ⁶¹Cu, ⁶²Cu, ⁶⁴Cu, ⁶⁷Cu, ⁶²Zn, ¹⁸⁸Re, ¹⁹⁸Au, ¹⁹⁹Au, ²²⁷Th, ¹¹¹Ag, ¹⁹⁹Ag, ²¹¹At, ²²³Ra, ⁸⁸Zr, and ⁸⁹Zr.
   or is a pharmaceutically acceptable salt thereof.

In certain embodiments of Formula III, R³ is H. In certain embodiments of Formula III, R³ is C₁-C₁₀ alkyl. In certain embodiments of Formula III, R³ is C₁-C₆ alkyl. In certain embodiments of Formula III, R³ is selected from methyl, ethyl, propyl, isopropyl, butyl, isobutyl, pentyl and hexyl. In certain embodiments of Formula III, R³ is methyl.

In certain embodiments of Formula III, L is a divalent linker as described above for Formula I.

In certain embodiments of Formula III, L is up to 20 atoms in length. In certain embodiments of Formula III, L is up to 15 atoms in length. In certain embodiments of Formula III, L is up to 10 atoms in length. In certain embodiments of Formula III, L is up to 5 atoms in length.

In certain embodiments of Formula III, L comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula III, L comprises one or more —N(R²)—, wherein R², independently for each occurrence, is H or C₁₋₁₀ alkyl, e.g., methyl.

In certain embodiments of Formula III, L comprises one or more —C(═O)— groups.

In certain embodiments of Formula III, L comprises one or more C₁₋₁₀ alkylene groups.

In certain embodiments of Formula III, L comprises one or more —N(R²)— groups and one or more —C(═O)— groups. In certain embodiments of Formula III, L comprises one or more —N(R²)— groups and one or more C₁₋₁₀ alkylene groups. In certain embodiments of Formula III, L comprises one or more —C(═O)— groups and one or more C₁₋₁₀ alkylene groups. In certain embodiments of Formula III, L comprises one or more —N(R²)— groups, one or more —C(═O)— groups, and one or more C₁₋₁₀ alkylene groups.

In certain embodiments of Formula III, R³, together with a moiety in L, form a C₂₋₉ heterocycle with the nitrogen atom(s) to which they are attached. In certain embodiments of Formula III, the C₂₋₉ heterocycle is a 5-, 6-, or 7-membered heterocycle. In certain embodiments of Formula III, the C₂₋₉ heterocycle is a 5-membered heterocycle selected from a pyrrolidine, pyrazolidine, and imidazoline. In certain embodiments of Formula III, the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane. In certain embodiments of Formula III, the C₂₋₉ heterocycle is a piperazine.

In certain embodiments of Formula III, M is selected from ⁶¹Cu, ⁶²Cu, ⁶⁴Cu, and ⁶⁷Cu.

In certain embodiments of Formula III, R³ is H; L is up to 20 atoms in length and comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; and M is selected from ⁶¹Cu, ⁶²Cu, ⁶⁴Cu, and ⁶⁷Cu. In certain embodiments of Formula III, R³ is H; L is up to 20 atoms in length and comprises one or more occurrences of groups selected from —N(R²)—, —O—, —C(═O)—, C₁₋₁₀ alkylene, and combinations thereof, wherein R², independently and or each occurrence, is selected from H and C₁₋₁₀ alkyl; and M is selected from ⁶¹Cu, ⁶²Cu, ⁶⁴Cu, and ⁶⁷Cu.

In certain embodiments of Formula III, R³ is H; L is up to 20 atoms in length and comprises one or more —N(R²)— groups, wherein R², independently for each occurrence, is H or C₁₋₁₀ alkyl; and M is selected from ⁶¹Cu, ⁶²Cu, ⁶⁴Cu, and ⁶⁷Cu.

In certain embodiments of Formula III, R³ is H, L is up to 20 atoms in length and comprises one or more —C(═O)— groups, and M is selected from ⁶¹Cu, ⁶²Cu, ⁶⁴Cu, and ⁶⁷Cu.

In certain embodiments of Formula III, R³ is H; L is up to 20 atoms in length and comprises one or more or more C₁₋₁₀ alkylene groups; and M is selected from ⁶¹Cu, ⁶²Cu, ⁶⁴Cu, and ⁶⁷Cu.

In certain embodiments of Formula III, R³ is H and L is up to 20 atoms in length and comprises one or more —C(═O)— groups and one or more or more C₁₋₁₀ alkylene groups. In certain embodiments of Formula III, R³ is H and L is up to 20 atoms in length and comprises one or more —C(═O)— groups and one or more —N(R²)— groups, wherein R², independently for each occurrence, is H or C₁₋₁₀ alkyl. In certain embodiments of Formula III, R³ is H and L is up to 20 atoms in length and comprises one or more C₁₋₁₀ alkylene groups and one or more —N(R²)— groups, wherein R², independently for each occurrence, is H or C₁₋₁₀ alkyl. In certain embodiments of Formula III, R³ is H and L is up to 20 atoms in length and comprises one or more —C(═O)— groups, one or more C₁₋₁₀ alkylene groups, and one or more —N(R²)— groups, wherein R², independently for each occurrence, is H or C₁₋₁₀ alkyl.

In certain embodiments of Formula III, R³, together with a moiety in L, form a C₂₋₉ heterocycle with the nitrogen atom(s) to which they are attached, wherein L is up to 20 atoms in length and comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl, and M is selected from ⁶¹Cu, ⁶²Cu, ⁶⁴Cu, and ⁶⁷Cu.

In certain embodiments of Formula III, R³, together with a moiety in L, form a C₂₋₉ heterocycle with the nitrogen atom(s) to which they are attached, wherein L is up to 20 atoms in length and comprises one or more occurrences of groups selected from —N(R²)—, —O—, —C(═O)—, C₁₋₁₀ alkylene, and combinations thereof, wherein R², independently for each occurrence, is selected from H and C₁₋₁₀ alkyl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl, and M is selected from ⁶¹Cu, ⁶²Cu, ⁶⁴Cu, and ⁶⁷Cu.

In certain embodiments of Formula III, R³, together with a moiety in L, form a C₂₋₉ heterocycle with the nitrogen atom(s) to which they are attached, wherein L is up to 20 atoms in length and comprises one or more occurrences of-N(R²)—, wherein R², independently for each occurrence, is H or C₁₋₁₀ alkyl, and M is selected from ⁶¹Cu, ⁶²Cu, ⁶⁴Cu, and ⁶⁷Cu.

In certain embodiments of Formula III, the compound is a compound of Formula IIIa:

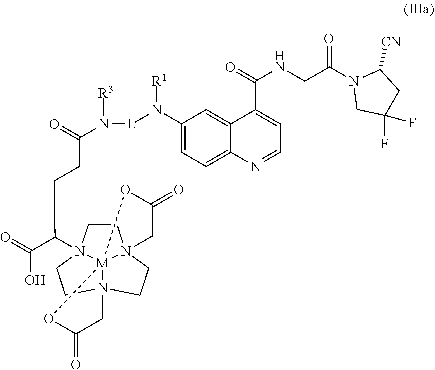

wherein:
R¹ is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; and
R³, L, and M are as described above for Formula III;
   or is a pharmaceutically acceptable salt thereof.

In certain embodiments of Formula IIIa, R¹ is H. In certain embodiments of Formula IIIa, R¹ is C₁-C₁₀ alkyl. In certain embodiments of Formula IIIa, R¹ is C₁-C₆ alkyl. In certain embodiments of Formula IIIa, R¹ is selected from methyl, ethyl, propyl, isopropyl, butyl, isobutyl, pentyl and hexyl. In certain embodiments of Formula IIIa, R¹ is methyl.

In certain embodiments of Formula IIIa, R³ is H. In certain embodiments of Formula IIIa, R³ is C₁-C₁₀ alkyl. In certain embodiments of Formula IIIa, R³ is C₁-C₆ alkyl. In certain embodiments of Formula IIIa, R³ is selected from methyl, ethyl, propyl, isopropyl, butyl, isobutyl, pentyl and hexyl. In certain embodiments of Formula IIIa, R³ is methyl.

In certain embodiments of Formula IIIa, R³, together with a moiety in L, form a C₂₋₉ heterocycle with the nitrogen atom(s) to which they are attached. In certain embodiments of Formula IIIa, the C₂₋₉ heterocycle is a 5-, 6-, or 7-membered heterocycle. In certain embodiments of Formula IIIa, the C₂₋₉ heterocycle is a 5-membered heterocycle selected from a pyrrolidine, pyrazolidine, and imidazoline. In certain embodiments of Formula IIIa, the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane. In certain embodiments of Formula IIIa, the C₂₋₉ heterocycle is a piperazine.

In certain embodiments of Formula IIIa, L is a divalent linker as described above for Formula I.

In certain embodiments of Formula IIIa, L is up to 20 atoms in length. In certain embodiments of Formula IIIa, L is up to 15 atoms in length. In certain embodiments of Formula IIIa, L is up to 10 atoms in length. In certain embodiments of Formula IIIa, L is up to 5 atoms in length.

In certain embodiments of Formula IIIa, L comprises one or more occurrences of groups selected from —N(R²)—, —O—, —S—, —C(═NR₂)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of Formula IIIa, L comprises one or more —N(R²)—, wherein R², independently for each occurrence, is H or C₁₋₁₀ alkyl, e.g., methyl.

In certain embodiments of Formula IIIa, L comprises one or more —C(═O)— groups.

In certain embodiments of Formula IIIa, L comprises one or more C₁₋₁₀ alkylene groups.

In certain embodiments of Formula IIIa, L comprises one or more —N(R²)— groups and one or more —C(═O)— groups. In certain embodiments of Formula IIIa, L comprises one or more —N(R²)— groups and one or more C₁₋₁₀ alkylene groups. In certain embodiments of Formula IIIa, L comprises one or more —C(═O)— groups and one or more C₁₋₁₀ alkylene groups. In certain embodiments of Formula IIIa, L comprises one or more —N(R²)— groups, one or more —C(═O)— groups, and one or more C₁₋₁₀ alkylene groups.

In certain embodiments of Formula IIIa, R³, together with a moiety in L, form a C₂₋₉ heterocycle with the nitrogen atom(s) to which they are attached. In certain embodiments of Formula IIIa, the C₂₋₉ heterocycle is a 5-, 6-, or 7-membered heterocycle. In certain embodiments of Formula IIIa, the C₂₋₉ heterocycle is a 5-membered heterocycle selected from a pyrrolidine, pyrazolidine, and imidazoline. In certain embodiments of Formula IIIa, the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane. In certain embodiments of Formula IIIa, the C₂₋₉ heterocycle is a piperazine.

In certain embodiments of Formula IIIa, M is selected from ⁶¹Cu, ⁶²Cu, ⁶⁴Cu, and ⁶⁷Cu.

In certain embodiments of Formula IIIa, R¹ is H or C₁₋₁₀ alkyl; R³ is H; L is up to 20 atoms in length and comprises one or more occurrences of groups selected from —N(R²)—, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR₁₋₁₀ COR₁₋₁₀, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; and M is selected from ⁶¹Cu, ⁶²Cu, ⁶⁴Cu, and ⁶⁷Cu. In certain embodiments of Formula IIIa, R¹ is H or C₁₋₁₀ alkyl; R³ is H; L is up to 20 atoms in length and comprises one or more occurrences of groups selected from —N(R²)—, —O—, —C(═O)—, C₁₋₁₀ alkylene, and combinations thereof, wherein R², independently and or each occurrence, is selected from H and C₁₋₁₀ alkyl; and M is selected from ⁶¹Cu, ⁶²Cu, ⁶⁴Cu, and ⁶⁷Cu.

In certain embodiments of Formula IIIa, R¹ is H or C₁₋₁₀ alkyl; R³ is H; L is up to 20 atoms in length and comprises one or more —N(R²)— groups, wherein R², independently for each occurrence, is H or C₁₋₁₀ alkyl; and M is selected from ⁶¹Cu, ⁶²Cu, ⁶⁴Cu, and ⁶⁷Cu.

In certain embodiments of Formula IIIa, R¹ is H or C₁₋₁₀ alkyl; R³ is H; and L is up to 20 atoms in length and comprises one or more —C(═O)— groups; and M is selected from ⁶¹Cu, ⁶²Cu, ⁶⁴Cu, and ⁶⁷Cu.

In certain embodiments of Formula IIIa, R¹ is H or C₁₋₁₀ alkyl; R³ is H; L is up to 20 atoms in length and comprises one or more or more C₁₋₁₀ alkylene groups; and M is selected from ⁶¹Cu, ⁶²Cu, ⁶⁴Cu, and ⁶⁷Cu.

In certain embodiments of Formula IIIa, R¹ is H or C₁₋₁₀ alkyl; R³ is H; and L is up to 20 atoms in length and comprises one or more —C(═O)— groups and one or more C₁₋₁₀ alkylene groups. In certain embodiments of Formula IIIa, R¹ is H or C₁₋₁₀ alkyl; R³ is H; and L is up to 20 atoms in length and comprises one or more —C(═O)— groups and one or more —N(R²)— groups, wherein R², independently for each occurrence, is H or C₁₋₁₀ alkyl. In certain embodiments of Formula IIIa, R¹ is H or C₁₋₁₀ alkyl; R³ is H; and L is up to 20 atoms in length and comprises one or more C₁₋₁₀ alkylene groups and one or more —N(R²)— groups, wherein R², independently for each occurrence, is H or C₁₋₁₀ alkyl. In certain embodiments of Formula IIIa, R¹ is H or C₁₋₁₀ alkyl; R³ is H; and L is up to 20 atoms in length and comprises one or more —C(═O)— groups, one or more C₁₋₁₀ alkylene groups, and one or more —N(R²)— groups, wherein R², independently for each occurrence, is H or C₁₋₁₀ alkyl.

In certain embodiments of Formula IIIa, R¹ is H or C₁₋₁₀ alkyl; R³, together with a moiety in L, form a C₂₋₉ heterocycle with the nitrogen atom(s) to which they are attached, wherein L is up to 20 atoms in length and comprises one or more occurrences of groups selected from —N(R²)_, —O—, —S—, —C(═NR²)—, —C(═O)—, C₁₋₁₀ alkylene, C₂₋₁₀ alkenylene, C₃₋₁₀ alkynylene, C₄₋₁₀ cycloalkylene, C₆₋₁₀ arylene, and combinations thereof, wherein R², independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; and M is selected from ⁶¹Cu, ⁶²Cu, ⁶⁴Cu, and ⁶⁷Cu.

In certain embodiments of Formula IIIa, R¹ is H or C₁₋₁₀ alkyl; R³, together with a moiety in L, form a C₂₋₉ heterocycle with the nitrogen atom(s) to which they are attached, wherein L is up to 20 atoms in length and comprises one or more occurrences of groups selected from —N(R²)—, —O—, —C(═O)—, C₁₋₁₀ alkylene, and combinations thereof, wherein R², independently for each occurrence, is selected from H and C₁₋₁₀ alkyl, optionally substituted by one or more substituents selected from —OH, —OR′, ═O, ═S, —SH, —SR′, —NH₂, —NHR′, —N(R′)₂, —NHCOR′, NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl; and M is selected from ⁶¹Cu, ⁶²Cu, ⁶⁴Cu, and ⁶⁷Cu.

In certain embodiments of Formula IIIa, R¹ is H or C₁₋₁₀ alkyl; R³, together with a moiety in L, form a C₂₋₉ heterocycle with the nitrogen atom(s) to which they are attached, wherein L is up to 20 atoms in length and comprises one or more occurrences of-N(R²)—, wherein R², independently for each occurrence, is H or C₁₋₁₀ alkyl; and M is selected from ⁶¹Cu, ⁶²Cu, ⁶⁴Cu, and ⁶⁷Cu.

### 3. Compositions

In another aspect, the present disclosure provides compositions (e.g., pharmaceutical compositions) comprising one or more of the compounds of Formula I-III described hereinabove and one or more pharmaceutically acceptable excipients.

Pharmaceutical compositions can be prepared in a manner well known in the pharmaceutical art. The excipient(s) must be acceptable in the sense of being compatible with the other ingredients of the composition and not deleterious to the recipient thereof. In accordance with another aspect of the disclosure there is also provided a process for the preparation of a pharmaceutical composition including a provided compound or pharmaceutically acceptable salt thereof, with one or more pharmaceutically acceptable excipients. The pharmaceutical composition can be for use in the diagnosis, treatment and/or prophylaxis of any of the conditions described herein.

Generally, a provided pharmaceutical composition is administered in an effective amount. The amount administered will typically be determined by a physician, in the light of the relevant circumstances, including the condition to be treated (e.g., a therapeutically effective amount) or image to be generated (e.g., diagnostically effective amount), the chosen route of administration, the pharmaceutical composition administered, the age, weight, and response of the individual patient, the severity of the patient's symptoms, and the like.

Pharmaceutical compositions may be presented in unit dose forms containing a predetermined amount of active ingredient per unit dose. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions.

Preferred unit dosage compositions are those containing a daily dose or sub-dose, or an appropriate fraction thereof, of an active ingredient. Such unit doses may therefore be administered once or more than once a day. Such pharmaceutical compositions may be prepared by any of the methods well known in the pharmacy art.

Pharmaceutical compositions may be adapted for administration by any appropriate route, for example by the oral (including buccal or sublingual), rectal, inhaled, intranasal, topical (including buccal, sublingual, or transdermal), vaginal or parenteral (including subcutaneous, intramuscular, intravenous, or intradermal) route. Such compositions may be prepared by any method known in the art of pharmacy, for example by bringing into association the active ingredient with the carrier(s) or excipient(s).

In certain embodiments, the pharmaceutical composition comprises a preservative. In certain embodiments, suitable preservatives may include, for example, methylparaben, propylparaben, sodium benzoate, and benzalkonium chloride.

In certain embodiments, the pharmaceutical composition comprises a buffering agent. In certain embodiments, suitable buffering agents may include, for example, citric acid, sodium citrate, phosphoric acid, potassium phosphate, and various other acids and salts.

In certain embodiments, the pharmaceutical composition is administered parenterally (e.g., subcutaneous, intravenous, intraarterial, intramuscular, intradermal, intraperitoneal, intrathecal, or intraocular).

The parenteral pharmaceutical compositions can be presented in unit-dose or multi-dose sealed containers, such as ampoules or vials, and can be stored in a freeze-dried (lyophilized) condition requiring only the addition of a sterile liquid excipient, for example, water for injections, immediately prior to use.

In certain embodiments, injectable pharmaceutical compositions are provided herein. The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Toissel, 4th ed, pages 622-630 (1986)).

In certain embodiments, pharmaceutical compositions according to the present disclosure are characterized by one or more of the activity and purity characteristics as described below.

Radioactivity

The term “radioactivity” (also referred to as activity, or total activity) is a physical quantity defined as the number of radioactive transformations per second that occur in a particular radionuclide. The unit of radioactivity used herein is the becquerel (symbol Bq), which is defined equivalent to reciprocal seconds (1/seconds or s⁻¹).

Molar Activity

The term “molar activity” is defined as the amount of radioactivity (e.g., number of nuclear disintegrations per second) per unit mole of radiolabeled compound, and is expressed in Bq/mol, e.g., GBq/mol and is used where the molecular weight of the labelled material is known.

In certain embodiments, the composition has a molar activity of 1 to 280 MBq/nmol, e.g., 5 to 265 MBq/nmol, 10 to 250 MBq/nmol, 15 to 235 MBq/nmol, 20 to 220 MBq/nmol, 25 to 205 MBq/nmol, 30 to 190 MBq/nmol, 35 to 175 MBq/nmol, 40 to 160 MBq/nmol, 45 to 150 MBq/nmol, 50 to 135 MBq/nmol, 55 to 120 MBq/nmol, 1 to 50 MBq/nmol, 2 to 48 MBq/nmol, 4 to 46 MBq/nmol, 6 to 44 MBq/nmol, 8 to 42 MBq/nmol, 10 to 40 MBq/nmol, 12 to 38 MBq/nmol, 14 to 36 MBq/nmol, 16 to 34 MBq/nmol, 18 to 32 MBq/nmol, 20 to 30 MBq/nmol, or 22 to 28 MBq/nmol. In certain embodiments, the composition has a molar activity of 24 MBq/nmol±3 MBq/nmol.

In certain embodiments, the composition has a molar activity of ≥35 MBq/nmol, ≥40 MBq/nmol, ≥45 MBq/nmol, ≥50 MBq/nmol, ≥55 MBq/nmol, ≥60 MBq/nmol, ≥65 MBq/nmol, ≥70 MBq/nmol, ≥75 MBq/nmol, ≥80 MBq/nmol, ≥85 MBq/nmol, ≥90 MBq/nmol, ≥95 MBq/nmol, ≥100 MBq/nmol, ≥105 MBq/nmol, ≥110 MBq/nmol, ≥115 MBq/nmol, ≥120 MBq/nmol, ≥125 MBq/nmol, ≥130 MBq/nmol, ≥135 MBq/nmol, ≥140 MBq/nmol, ≥145 MBq/nmol, ≥150 MBq/nmol, ≥155 MBq/nmol, ≥160 MBq/nmol, ≥165 MBq/nmol, ≥170 MBq/nmol, ≥175 MBq/nmol, ≥180 MBq/nmol, ≥185 MBq/nmol, ≥190 MBq/nmol, ≥195 MBq/nmol, or ≥200 MBq/nmol.

In certain embodiments, the composition has a molar activity of 1 to 250 MBq/nmol, for example, 1 to 200 MBq/nmol, 1 to 150 MBq/nmol, 1 to 100 MBq/nmol, 1 to 50 MBq/nmol, 50 to 250 MBq/nmol, 50 to 200 MBq/nmol, 50 to 150 MBq/nmol, 50 to 100 MBq/nmol, 100 to 250 MBq/nmol, 100 to 150 MBq/nmol, 150 to 250 MBq/nmol, 150 to 200 MBq/nmol, or 200 to 250 MBq/nmol. In certain embodiments, the composition is characterized by molar activity of 1 to 150 MBq/nmol.

In certain embodiments, the composition has a molar activity of ≥90 MBq/nmol, ≥88 MBq/nmol, ≥86 MBq/nmol, ≥84 MBq/nmol, ≥82 MBq/nmol, ≥80 MBq/nmol, ≥78 MBq/nmol, ≥76 MBq/nmol, ≥74 MBq/nmol, ≥72 MBq/nmol, ≥70 MBq/nmol, ≥68 MBq/nmol, ≥66 MBq/nmol, ≥64 MBq/nmol, ≥62 MBq/nmol, ≥60 MBq/nmol, ≥58 MBq/nmol, ≥56 MBq/nmol, ≥54 MBq/nmol, ≥52 MBq/nmol, ≥50 MBq/nmol, ≥48 MBq/nmol, ≥46 MBq/nmol, ≥44 MBq/nmol, or ≥42 MBq/nmol.

In certain embodiments, the composition has a molar activity of ≥3 MBq/nmol, ≥4 MBq/nmol, ≥5 MBq/nmol, ≥6 MBq/nmol, ≥7 MBq/nmol, ≥8 MBq/nmol, ≥9 MBq/nmol, ≥10 MBq/nmol, ≥11 MBq/nmol, ≥12 MBq/nmol, ≥13 MBq/nmol, ≥14 MBq/nmol, ≥15 MBq/nmol, ≥16 MBq/nmol, ≥17 MBq/nmol, ≥18 MBq/nmol, or ≥19 MBq/nmol.

In certain embodiments, the composition has a molar activity of ≥3 MBq/nmol, ≥5 MBq/nmol, ≥10 MBq/nmol, ≥15 MBq/nmol, ≥20 MBq/nmol, ≥25 MBq/nmol, ≥30 MBq/nmol, ≥35 MBq/nmol, ≥40 MBq/nmol, ≥45 MBq/nmol, ≥50 MBq/nmol, ≥55 MBq/nmol, ≥60 MBq/nmol, ≥65 MBq/nmol, ≥70 MBq/nmol, ≥75 MBq/nmol, ≥80 MBq/nmol, ≥85 MBq/nmol, ≥90 MBq/nmol, ≥95 MBq/nmol, ≥100 MBq/nmol, ≥105 MBq/nmol, ≥110 MBq/nmol, ≥115 MBq/nmol, ≥120 MBq/nmol, ≥125 MBq/nmol, 130 MBq/nmol, 135 MBq/nmol, 140 MBq/nmol, 145 MBq/nmol, 150 MBq/nmol, 155 MBq/nmol, ≥160 MBq/nmol, ≥165 MBq/nmol, ≥170 MBq/nmol, ≥175 MBq/nmol, ≥180 MBq/nmol, ≥185 MBq/nmol, ≥190 MBq/nmol, ≥195 MBq/nmol, ≥200 MBq/nmol, ≥205 MBq/nmol, ≥210 MBq/nmol, ≥215 MBq/nmol, 220≥MBq/nmol, ≥225 MBq/nmol, ≥230 MBq/nmol, ≥235 MBq/nmol, ≥240 MBq/nmol, ≥245 MBq/nmol, ≥250 MBq/nmol, ≥255 MBq/nmol, ≥260 MBq/nmol, ≥265 MBq/nmol, ≥270 MBq/nmol, ≥275 MBq/nmol, or ≥280 MBq/nmol. In certain embodiments, the composition has a molar activity of ≥24 MBq/nmol.

In certain embodiments, the composition has a molar activity of 1 to 250 MBq/nmol, for example, 1 to 200 MBq/nmol, 1 to 150 MBq/nmol, 1 to 100 MBq/nmol, 1 to 50 MBq/nmol, 50 to 250 MBq/nmol, 50 to 200 MBq/nmol, 50 to 150 MBq/nmol, 50 to 100 MBq/nmol, 100 to 250 MBq/nmol, 100 to 150 MBq/nmol, 150 to 250 MBq/nmol, 150 to 200 MBq/nmol, or 200 to 250 MBq/nmol.

Activity Concentration

Activity concentration is the total amount of radioactivity per unit volume. In certain embodiments, activity concentration is expressed in Bq/L or magnitudes thereof (e.g., MBq/mL).

In certain embodiments, a composition provided is characterized by an activity concentration of ≥8 MBq/mL. In certain embodiments, a composition provided herein is characterized by an activity concentration of 8 to 10 MBq/mL, 10 to 20 MBq/mL, 20 to 30 MBq/mL, 30 to 40 MBq/mL, 40 to 50 MBq/mL, 50 to 60 MBq/mL, 60 to 70 MBq/mL, 70 to 80 MBq/mL, 80 to 90 MBq/mL, 90 to 100 MBq/mL, 100 to 110 MBq/mL, 110 to 120 MBq/mL, 120 to 130 MBq/mL, 130 to 140 MBq/mL, 140 to 150 MBq/mL, 150 to 160 MBq/mL, 160 to 170 MBq/mL, 170 to 180 MBq/mL, 180 to 190 MBq/mL, 190 to 200 MBq/mL, 200 to 210 MBq/mL, 210 to 220 MBq/mL, 220 to 230 MBq/mL, 230 to 240 MBq/mL, 240 to 250 MBq/mL, 250 to 260 MBq/mL, 260 to 270 MBq/mL, 270 to 280 MBq/mL, 280 to 290 MBq/mL, 290 to 300 MBq/mL, 300 to 310 MBq/mL, 310 to 320 MBq/mL, 320 to 330 MBq/mL, 330 to 340 MBq/mL, 340 to 350 MBq/mL, 350 to 360 MBq/mL, 360 to 370 MBq/mL, 370 to 380 MBq/mL, 380 to 390 MBq/mL, 390 to 400 MBq/mL, 400 to 410 MBq/mL, 410 to 420 MBq/mL, 420 to 430 MBq/mL, 430 to 440 MBq/mL, 440 to 450 MBq/mL, 450 to 460 MBq/mL, 460 to 470 MBq/mL, 470 to 480 MBq/mL, 480 to 490 MBq/mL, 490 to 500 MBq/mL, 500 to 510 MBq/mL, 510 to 520 MBq/mL, 520 to 530 MBq/mL, 530 to 540 MBq/mL, 540 to 550 MBq/mL, 550 to 560 MBq/mL, 560 to 570 MBq/mL, 570 to 580 MBq/mL, 580 to 590 MBq/mL, 590 to 600 MBq/mL, 600 to 610 MBq/mL, 610 to 620 MBq/mL, 620 to 630 MBq/mL, 630 to 640 MBq/mL, 640 to 650 MBq/mL, 650 to 660 MBq/mL, 660 to 670 MBq/mL, 670 to 680 MBq/mL, 680 to 690 MBq/mL, 690 to 700 MBq/mL, 700 to 710 MBq/mL, 710 to 720 MBq/mL, 720 to 730 MBq/mL, 730 to 740 MBq/mL, 740 to 750 MBq/mL, 750 to 760 MBq/mL, 760 to 770 MBq/mL, 770 to 780 MBq/mL, 780 to 790 MBq/mL, 790 to 800 MBq/mL, 800 to 810 MBq/mL, 810 to 820 MBq/mL, 820 to 830 MBq/mL, 830 to 840 MBq/mL, 840 to 850 MBq/mL, 850 to 860 MBq/mL, 860 to 870 MBq/mL, 870 to 880 MBq/mL, 880 to 890 MBq/mL, 890 to 900 MBq/mL, 900 to 910 MBq/mL, 910 to 920 MBq/mL, 920 to 930 MBq/mL, 930 to 940 MBq/mL, 940 to 950 MBq/mL, 950 to 960 MBq/mL, 960 to 970 MBq/mL, 970 to 980 MBq/mL, 980 to 990 MBq/mL, or 990 to 1000 MBq/mL.

In certain embodiments, a composition provided has an activity concentration of ≥8 MBq/mL. In certain embodiments, a composition has an activity concentration of 5 to 500 MBq/mL, 20 to 480 MBq/mL, 40 to 460 MBq/mL, 60 to 440 MBq/mL, 80 to 420 MBq/mL, 100 to 400 MBq/mL, 120 to 380 MBq/mL, 140 to 360 MBq/mL, 160 to 340 MBq/mL, 180 to 320 MBq/mL, or 200 to 300 MBq/mL.

In certain embodiments, a composition has an activity concentration of ≥3 MBq/mL, ≥4 MBq/mL, ≥5 MBq/mL, ≥6 MBq/mL, ≥7 MBq/mL, ≥8 MBq/mL, ≥9 MBq/mL, ≥10 MBq/mL, ≥12 MBq/mL, ≥15 MBq/mL, ≥20 MBq/mL, ≥25 MBq/mL, ≥30 MBq/mL, ≥35 MBq/mL, 40 MBq/mL, ≥45 MBq/mL, ≥50 MBq/mL, ≥55 MBq/mL, ≥60 MBq/mL, ≥65 MBq/mL, ≥70 MBq/mL, ≥75 MBq/mL, ≥80 MBq/mL, ≥85 MBq/mL, ≥90 MBq/mL, ≥95 MBq/mL, ≥100 MBq/mL, ≥105 MBq/mL, ≥110 MBq/mL, ≥115 MBq/mL, ≥120 MBq/mL, ≥125 MBq/mL, 130 MBq/mL, 135 MBq/mL, 140 MBq/mL, 145 MBq/mL, 150 MBq/mL, 155 MBq/mL, ≥160 MBq/mL, ≥165 MBq/mL, ≥170 MBq/mL, ≥175 MBq/mL, ≥180 MBq/mL, ≥185 MBq/mL, ≥190 MBq/mL, ≥195 MBq/mL, ≥200 MBq/mL, ≥205 MBq/mL, ≥210 MBq/mL, ≥215 MBq/mL, 220≥MBq/mL, ≥225 MBq/mL, ≥230 MBq/mL, ≥235 MBq/mL, ≥240 MBq/mL, ≥245 MBq/mL, ≥250 MBq/mL, ≥255 MBq/mL, ≥260 MBq/mL, ≥265 MBq/mL, ≥270 MBq/mL, ≥275 MBq/mL, or ≥280 MBq/mL.

In certain embodiments, the activity concentration of the resulting pharmaceutical composition may be diluted (e.g., by a factor of 3 to 10), as long as the activity concentration is ≥8 MBq/mL. In certain embodiments, a composition has an activity concentration 8 to 20 MBq/mL, 9 to 19 MBq/mL, 10 to 18 MBq/mL, 11 to 19 MBq/mL, 12 to 18 MBq/mL, 13 to 15 MBq/mL, 14 to 15 MBq/mL, 8 to 14 MBq/mL, 8 to 13 MBq/mL, 8 to 12 MBq/mL, 8 to 11 MBq/mL, 8 to 10 MBq/mL, 8 to 9 MBq/mL, 9 to 14 MBq/mL, 10 to 13 MBq/mL, or 11 to 12 MBq/mL.

Radiochemical Purity

“Radiochemical purity,” as understood herein, is the ratio, given as a percent, of radioactivity from the desired radionuclide in the radiopharmaceutical composition (e.g., the desired radionuclide that is chelated in a radiotracer as described herein) to the total radioactivity of the composition that comprises the radiopharmaceutical. It is important to know that the majority of the radioactive isotope is attached to the tracer construct and is not free or attached to another chemical entity as these forms may have a different biodistribution. Radiochemical purity (RCP) measurements establish the content of impurities labelled with the same radionuclide used to prepare a radiopharmaceutical, but with a different chemical form. For most radiopharmaceuticals the lower limit of radiochemical purity is 95%, that is, at least 95% of the radioactive isotope must be attached to the ligand. Radiochemical purity determination can be carried out by a variety of chromatographic methods.

Radiochemical purity is determined according to methods well known to those of skill in the art, e.g., radio-HPLC, iTLC and/or γ-spectrometry. As is understood in the art, determination of radiochemical purity is not strictly quantitative, and it is calculated as the ratio between the peak area of the desired radiopharmaceutical and the overall area of all the detected peaks in the radiochromatogram (corrected for decay). The instrument used to determine radiochemical purity with HPLC (radio-HPLC) is a radiometric detector (radiodetector), which has an in-line detector connected in series with a UV or other physicochemical detector. The radiometric detector can be a Geiger-Müller probe, a scintillation detector, or a PIN diode. As compared with radio-HPLC it has the big advantage that all applied radioactivity is detected and there are no concerns with recovery.

In certain embodiments, the composition is characterized by a radiochemical purity of ≥90%. In certain embodiments, the composition is characterized by a radiochemical purity of ≥91%. In certain embodiments, the composition is characterized by a radiochemical purity of ≥95%, ≥96%, ≥97%, ≥98%, or ≥99%. In certain embodiments, the composition is characterized by a radiochemical purity of ≥90%. In certain embodiments, the composition is characterized by radiochemical purity of ≥95%. In certain embodiments, the composition is characterized by radiochemical purity of ≥96%. In certain embodiments, the composition is characterized by radiochemical purity of 98%.

In certain embodiments, the composition provided is characterized by a radiochemical purity of ≥94.0%, ≥94.5%, ≥95.0%, ≥95.5%, ≥96.0%, ≥96.5%, ≥97.0%, ≥97.5%, ≥98.0%, ≥98.5%, ≥99.0%, or ≥99.5%.

In certain embodiments, the composition provided is characterized by a radiochemical purity of ≥95.2%, ≥95.4%, ≥95.6%, ≥95.8%, ≥96%, ≥96.2%, ≥96.4%, ≥96.6%, ≥96.8%, ≥97%≥97.2%≥97.4%≥97.6%≥97.8%≥98%≥98.2%≥98.4%≥98.6%≥98.8%≥99%, ≥99.2%, ≥99.4%, ≥99.6%, or ≥99.8%.

Radionuclidic Purity

The term “radionuclidic purity” refers to the ratio, expressed as a percentage, of the radioactivity of the desired radionuclide to the total radioactivity of the sample, e.g., the starting material used to prepare a radiolabeled pharmaceutical. As reported herein, unless otherwise specified, radionuclidic purity is determined by high resolution gamma spectroscopy (e.g., high-purity germanium (HPGe) detector) on a sample after expiration, e.g. >8 hours or >3 weeks) and is then extrapolated (e.g., using the TENDLE-2019 database according to procedures well known in the art), and reported herein as the value at the end of synthesis (EoB+2 hours) of the radionuclide.

In certain embodiments, the composition is characterized by radionuclidic purity of the compound at end of synthesis≥85%, for example, of ≥86%, ≥87%, ≥88%, ≥89%, ≥90%, ≥91%, ≥92%, ≥93%, ≥94%, ≥95%, ≥96%, ≥97%, ≥98%, or of ≥99%.

In certain embodiments, the composition is characterized by radionuclidic purity of the compound at end of synthesis≥90.5%, e.g., ≥91%, ≥91.5%, ≥92%, ≥92.5%, ≥93%, ≥93.5%, ≥94%≥94.5%, 95% 95.5%, ≥96%≥96.5%≥97%≥97.5%≥98%≥98.5%≥99% or ≥99.5%.

In certain embodiments, the composition is characterized by a radionuclidic purity of ≥95.1%, e.g., ≥95.2%, ≥95.3%, ≥95.4%, ≥95.5%, ≥95.6%, ≥95.7%, ≥95.8%, ≥95.9%, ≥96%, ≥96.1%, ≥96.2%, ≥96.3%, ≥96.4%, ≥96.5%, ≥96.6%, ≥96.7%, ≥96.8%, ≥96.9%, ≥97%, ≥97.1%, ≥97.2%, ≥97.3%, ≥97.4%, ≥97.5%, ≥97.6%, ≥97.7%, ≥97.8%, ≥97.9%, ≥98%, ≥98.1%, ≥98.2%, ≥98.3%, ≥98.4%, ≥98.5%, ≥98.6%, ≥98.7%, ≥98.8%, ≥98.9%, ≥99%, ≥99.1%, ≥99.2%, ≥99.3%, ≥99.4%, ≥99.5%, ≥99.6%, ≥99.7%, ≥99.8%, or ≥99.9%.

In certain embodiments, the composition is characterized by radionuclidic purity of ≥97% (at end of synthesis). In certain embodiments, the composition is characterized by radionuclidic purity of ≥93%, ≥94%, ≥95%, ≥96%, ≥98%, or ≥99% (at end of synthesis).

### 4. Methods of Use

In one aspect, the present disclosure provides compounds and pharmaceutical compositions comprising the same for use in medicine, i.e., for use in treatment, imaging, diagnosing, companion diagnosing, etc. The present disclosure further provides the use of any compounds or pharmaceutical compositions described herein for targeted radiotherapy, which would be beneficial to diagnose and/or treat cancer.

In certain embodiments, the compounds or pharmaceutical compositions of the present disclosure are administered to a subject once a day, twice a day, daily, or every other day. In certain embodiments, the compounds or pharmaceutical compositions of the present disclosure are administered to a subject twice a week, once a week, every ten days, every two weeks, every three weeks, every four weeks, once a month, every six weeks, every eight weeks, every three months, every four months, every six months, every eight months, every nine months, or annually. The dosage and frequency (single or multiple doses) of compound or pharmaceutical composition administered can vary depending upon a variety of factors, including route of administration; size, age, sex, health, body weight, body mass index, and diet of the recipient; nature and extent of the symptoms of the disease being treated (e.g., the disease responsive treatment) and complications from any disease or treatment regimen. Other therapeutic regimens or agents can be used in conjunction with the methods and compounds of the invention.

For any provided compound or pharmaceutical composition, the effective amount (e.g., the diagnostically effective or therapeutically effective amount) can be initially determined from cell culture assays and/or animal testing. Target concentrations will be those concentrations of active compound(s) that are capable of diagnosing, monitoring, and/or treating cancer in a patient or subject.

Therapeutic efficacy of the compound may be determined from animal models. The dosage in humans can be adjusted during the clinical trials via dose escalation studies by monitoring safety and efficacy.

Dosages may be varied depending upon the requirements of the patient and the compound or pharmaceutical composition being employed. The dose administered to a patient, in the context of the present invention, should be sufficient to affect a beneficial therapeutic response in the patient over time. The size of the dose also will be determined by the existence, nature, and extent of any adverse side effects.

In one aspect, compounds provided herein display one or more improved pharmacokinetic (PK) properties (e.g., Cₘₐₓ, tₘₐₓ, Cₘᵢₙ, t_{1/2}, AUC, CL, bioavailability, etc.) when compared to a reference compound.

In some embodiments, a compound of the disclosure or a pharmaceutical composition comprising the same is provided as a unit dose.

4.4.1. Imaging and Diagnosis

In an aspect of the present disclosure, methods of generating an image of a subject (e.g., of a certain region or part of the subject's body) are provided, the method comprising administering to the subject a compound described herein comprising a radionuclide. In certain embodiments, the radionuclide is a metal radio nuclide. In certain embodiments, the radionuclide is selected from ⁶⁰cu, ⁶¹Cu, ⁶²Cu, ⁶⁴Cu, and ⁶⁷Cu. In certain embodiments, the radionuclide is ⁶¹Cu. In certain embodiments, the radionuclide is ⁶⁷Cu.

In certain embodiments, methods of generating one or more images of a subject are provided (e.g., of a certain region or part of the subject's body) comprising administering to the subject an effective amount of a compound comprising a radionuclide described herein, or a pharmaceutical composition comprising the same, and generating one or more images of at least a part of the subject's body. In certain embodiments, two or more images of a subject are generated, such as, for example, three or more images, four or more images, or five or more images. In certain embodiments, a diagnostically effective amount of the compound comprising a radionuclide or pharmaceutical composition comprising the same is administered to the subject, i.e., an amount sufficient to identify (visually or computationally) localization of the radionuclide within regions or parts of the subject's body. In some embodiments, the radionuclide is a metal radionuclide. In certain embodiments, the radionuclide is selected from ⁶⁰Cu, ⁶¹Cu, ⁶²Cu, ⁶⁴Cu, and ⁶⁷Cu. In some embodiments, the radionuclide is ⁶¹Cu.

In certain embodiments, the one or more images are generated using positron emission tomography (PET). In certain embodiments, the one or more images are generated using PET-computer tomography (PET-CT). In certain embodiments, the one or more images are generated using single-photon emission computerized tomography (SPECT).

In certain embodiments, the image is generated using PET or PET-CT, wherein the radionuclide is ⁶¹Cu. In certain embodiments, the image is generated using SPECT wherein the radionuclide is ⁶¹Cu or ⁶⁷Cu.

In certain embodiments, after the one or more images are generated, the method further comprises determining the presence or absence of a disease in a subject based on the presence or absence of localization of the radionuclide in the one or more images of the subject's body.

In certain embodiments, the disease is cancer. In certain embodiments, the cancer is selected from breast cancer (e.g., triple-negative breast cancer), pancreatic cancer, small intestine cancer, colon cancer, gastric cancer, rectal cancer, lung cancer (e.g., non-small cell lung cancer), head and neck cancer, ovarian cancer, hepatocellular carcinoma, epithelial cancer, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer.

In certain embodiments, the disease is selected from cardiovascular diseases, liver fibrosis and cirrhosis, arthritic disorders (e.g., rheumatoid arthritis), IgG4-related disease, pulmonary fibrosis and interstitial lung disease, Crohn's disease, tuberculosis, sarcoidosis, and periprosthetic joint infections.

In another aspect of the present disclosure, a method of detecting a disease in a subject is provided, the method comprising administering to a subject an effective amount of a compound comprising a radionuclide described herein or pharmaceutical composition comprising the same; detecting the localization of the radionuclide in the subject using, e.g., PET, PET-CT, or SPECT; and determining the presence or absence of the disease based on the presence or absence of localization. In some embodiments, the radionuclide is a metal radionuclide. In certain embodiments, the radionuclide is selected from ⁶⁰Cu, ⁶¹Cu, ⁶²Cu, ⁶⁴Cu, and ⁶⁷Cu. In some embodiments, the radionuclide is ⁶¹Cu.

In certain embodiments, the disease to be detected is any disease which overexpresses FAP, e.g., cancers, inflammatory diseases, infectious diseases, and immune diseases.

In certain embodiments, the disease is cancer. In certain embodiments, the cancer is selected from breast cancer (e.g., triple-negative breast cancer), pancreatic cancer, small intestine cancer, colon cancer, gastric cancer, rectal cancer, lung cancer (e.g., non-small cell lung cancer), head and neck cancer, ovarian cancer, hepatocellular carcinoma, epithelial cancer, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer.

In certain embodiments, the disease is selected from cardiovascular diseases, liver fibrosis and cirrhosis, arthritic disorders (e.g., rheumatoid arthritis), IgG4-related disease, pulmonary fibrosis and interstitial lung disease, Crohn's disease, tuberculosis, sarcoidosis, and periprosthetic joint infections.

In another aspect of the present disclosure, a method of monitoring the effect of cancer treatment on a subject afflicted with cancer is provided. The method comprises administering to a subject an effective amount of a compound comprising a radionuclide described herein or a pharmaceutical composition comprising the same; detecting localization of the radionuclide in the subject using, e.g., PET, PET-CT, or SPECT; and determining the effects of the cancer treatment.

In certain embodiments, the compound comprising a radionuclide or pharmaceutical composition comprising the same is administered to the subject and localization is observed at multiple time points, i.e., at an earlier time point (e.g., before cancer treatment begins (t=0)) and at a later time point, e.g., 2 weeks after commencing treatment, 3 weeks after commencing treatment, 1 month after commencing treatment, 2 months after commencing treatment, 3 months after commencing treatment, 4 months after commencing treatment, 5 months after commencing treatment, or 6 or more months after commencing treatment. In certain but not all embodiments, the cancer treatment is determined to be beneficial (i.e., a positive effect) if less localization is observed at the later time point compared to the earlier time point. In certain but not all embodiments, the cancer treatment is determined to not be beneficial (i.e., a negative effect) if more localization is observed at the later time point compared to the earlier time point. In certain but not all embodiments, the cancer treatment is determined to not have an effect if there is no difference in localization at the later time point compared to the earlier time point.

4.4.2. Therapy

In an aspect of the present disclosure, a method of treating a disease in a patient afflicted with a disease is provided, the method comprising administering to the patient an effective amount of compound or pharmaceutical composition described herein.

In certain embodiments, the compound administered is of Formula I, wherein T comprises a drug that is not a radionuclide and does not contain a radionuclide. Such embodiments are useful for treating inflammatory diseases, infectious diseases, and immune diseases. In certain embodiments, the disease is selected from cardiovascular diseases, liver fibrosis and cirrhosis, arthritic disorders (e.g., rheumatoid arthritis), IgG4-related disease, pulmonary fibrosis and interstitial lung disease, Crohn's disease, tuberculosis, sarcoidosis, and periprosthetic joint infections.

In certain embodiments, the compound administered is of Formula I, wherein T comprises a drug that is cytotoxic. In certain embodiments, the compound comprises a radionuclide selected from ⁶⁴Cu and ⁶⁷Cu. Such embodiments are useful in treating cancers, e.g., breast cancer (e.g., triple-negative breast cancer), pancreatic cancer, small intestine cancer, colon cancer, gastric cancer, rectal cancer, lung cancer (e.g., non-small cell lung cancer), head and neck cancer, ovarian cancer, hepatocellular carcinoma, epithelial cancer, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer.

4.4.3. Theranostics

In an aspect of the present disclosure, a theranostic method comprises the use of a pair of *Cu radiotracers (“theranostic pair”), as provided herein, for both imaging/diagnosis of a disease and for treating the disease in the same patient, wherein the theranostic pair of radiotracers differ only in the radionuclide, i.e., they are different radioisotopes. In certain embodiments, the theranostic pair comprises a γ or positron emitting radionuclide in the radiotracer for imaging/diagnosis (e.g., with PET, PET-CT, or SPECT) and a R emitting radionuclide in the radiotracer for therapy.

In certain embodiments, the theranostic pair comprises ⁶¹Cu (for imaging/diagnosis) and ⁶⁷Cu (for therapy). In certain embodiments, this is referred to as a ^{61/67}Cu theranostic pair.

Certain embodiments of the theranostic method comprise the administration of a diagnostic form of the radiotracer (e.g., wherein *Cu is ⁶¹Cu for PET or wherein *Cu is ⁶⁷Cu for SPECT), enabling expression of the therapeutic target to be visualized in vivo with a companion imaging method before switching to the radiolabeled therapeutic counterpart, e.g., wherein *Cu is ⁶⁴Cu or ⁶⁷Cu.

In certain embodiments, a theranostic method comprises:
(a) administering to a subject an effective amount of a compound comprising a ⁶¹Cu radionuclide described herein or a pharmaceutical composition comprising the same;
(b) generating one or more images of the subject (e.g., of a certain region or part of the subject's body); and
(c) administering to the subject an effective amount of a compound comprising a ⁶⁷Cu radionuclide described herein or a pharmaceutical composition comprising the same, wherein the compounds of step (a) and (c) differ only in radioisotopic identity.

In certain embodiments, the amount of compound comprising a ⁶¹Cu radionuclide described herein or pharmaceutical composition comprising the same administered in step (a) is effective to generate one or more images of subject (i.e., a “detectably effective amount”). In certain embodiments, the amount of compound comprising a ⁶¹Cu radionuclide described herein or pharmaceutical composition comprising the same administered in step (a) is effective to diagnose the presence or absence of a disease (i.e., a “diagnostically effective amount”).

In certain embodiments, the method further comprises determining, via the one or more images of the subject, the presence or absence of a disease in the subject based on the presence or absence of localization of the ⁶¹Cu radionuclide in the subject's body. In instances where the subject is not determined to have a disease, step (c) in the method is not performed.

In certain embodiments, the amount of compound comprising a ⁶⁷Cu radionuclide described herein or a pharmaceutical composition comprising the same administered in step (c) is effective to treat the disease in the subject (i.e., a “therapeutically effective amount”).

In certain embodiments, a theranostic method comprises:
(a) generating one or more images of a subject (e.g., of a certain region or part of the subject's body) comprising administering to the subject an effective amount of a compound comprising a ⁶¹Cu radionuclide described herein or a pharmaceutical composition comprising the same;
(b) determining, via the one or more images of the subject, the presence or absence of a disease in the subject based on the presence or absence of localization of the ⁶¹Cu radionuclide in the subject's body; and
(c) administering to the subject, when the presence of a disease in the subject is determined, an effective amount of a compound comprising a ⁶⁷Cu radionuclide described herein, or a pharmaceutical composition comprising the same, wherein the compounds in step (a) and (c) differ only in the radionuclide identity.

### 5. ENUMERATED EMBODIMENTS

Embodiment 1. A compound, wherein the compound is of Formula I:

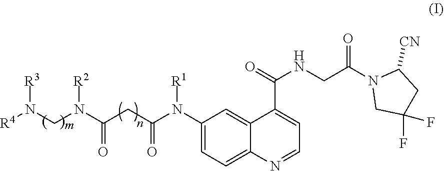

wherein:
R¹ is R^{a};
R² and R³ are each R^{a} or together form a C₂₋₉ heterocycle with the nitrogen atoms to which they are attached;
R⁴ is H, an amine protecting group, or -L-T;
R^{a}, independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl;
L is a bond or a divalent linker;
T comprises (a) a chelating moiety suitable for chelating a radionuclide, (b) an imaging agent, or (c) a drug;
n is an integer from 1 to 20; and
m is an integer from 1 to 20;
   or is a pharmaceutically acceptable salt thereof.
   Embodiment 2. The compound of embodiment 1, wherein R¹ is methyl or H.
   Embodiment 3. The compound of embodiment 1 or 2, wherein R² is H and R³ is H.
   Embodiment 4. The compound of embodiment 1 or 2, wherein R² and R³ together form a C₂₋₉ heterocycle with the nitrogen atoms to which they are attached.
   Embodiment 5. The compound of embodiment 4, wherein the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane.
   Embodiment 6. The compound of any one of embodiments 1-5, wherein R⁴ is H.
   Embodiment 7. The compound of any one of embodiments 1-5, wherein R⁴ is an amine protecting group.
   Embodiment 8. The compound of any one of embodiments 1-5, wherein R⁴ is -L-T.
   Embodiment 9. The compound of embodiment 8, wherein L is a divalent linker selected from an acid-labile linker, a hydrolysis-labile linker, an enzymatically cleavable linker, a reduction labile linker, a self-immolative linker, and a non-cleavable linker.
   Embodiment 10. The compound of embodiment 8 or 9, wherein T comprises a chelating moiety suitable for chelating a radionuclide.
   Embodiment 11. The compound of embodiment 10, wherein the chelating moiety is chelated to a radionuclide, and the radionuclide is selected from alpha radiation emitting isotopes, beta radiation emitting isotopes, gamma radiation emitting isotopes, Auger electron emitting isotopes, X-ray emitting isotopes, and fluorescence emitting isotopes.
   Embodiment 12. The compound of embodiment 11, wherein the radionuclide is selected from ²²⁵Ac, ⁵¹Cr, ⁶⁶Ga, ⁶⁷Ga, ⁶⁸GA, [¹⁸F]AlF, ¹¹¹In, ^{113m}In, ^{52m}Mn, ^{99m}Tc, ¹⁸⁶Re, ¹⁸⁸Re, ¹³⁹La, ¹⁴⁰La, ¹⁷⁵Yb, ¹⁷⁹Yb, ¹⁵³Sm, ^{177m}Sn, ¹⁶⁶Ho, ⁸⁶Y, ⁸⁸Y, ⁹⁰Y, ¹⁴⁹Pm, ¹⁶⁵Dy, ¹⁶⁹Er, ¹⁷⁷Lu, ⁵²Fe, ⁴³Sc, ⁴⁴Sc, ⁴⁶Sc, ⁴⁷Sc, ¹⁴²Pr, ¹⁵⁷Gd, ¹⁵⁹Gd, ²¹²Bi, ²¹³Bi, ⁷²As, ⁷⁷As, ⁹⁷Ru, ¹⁰⁹Pd, ¹⁰⁵Rh, ^{101m}Rh, ¹¹⁹Sb, ¹⁹⁷Hg, ¹⁵¹Eu, ¹⁵³Eu, ¹⁶⁹Eu, ²⁰¹Tl, ¹⁴⁹Tb, ¹⁵²Tb, ¹⁵⁵Tb, ¹⁶¹Tb, ²⁰³Pb, ²¹²Pb, ¹⁵¹Pm, ¹⁵³Pm, ¹⁴²Pr, ¹⁴³Pr, ⁵⁵Co, ⁶⁰Cu, ⁶¹Cu, ⁶²Cu, ⁶⁴Cu, ⁶⁷Cu, ⁶²Zn, ¹⁸⁸Re, ¹⁹⁸Au, ¹⁹⁹Au, ²²⁷Th, ¹¹¹Ag, ¹⁹⁹Ag, ²¹¹At, ²²³Ra, ⁸⁸Zr, and ⁸⁹Zr.
   Embodiment 13. The compound of embodiment 12, wherein the radionuclide is selected from ⁶¹Cu, ⁶⁴Cu, and ⁶⁷Cu.
   Embodiment 14. The compound of any one of embodiments 10-13, wherein the chelating moiety is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate), NODAGA (1,4,7-triazacyclononane-1-glutaric acid-4,7-acetic acid) (also known as NOTAGA), NODA Deferoxamine (1,4,7-triazacyclononane-1,4-diyl)diacetic acid DFO), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), and 4,4′-((3,6,10,13,16,19-hexaazabicyclo[6.6.6]ico-sane-1,8-diylbis(aza-nediyl))bis(methylene))dibenzoic acid (BaBaSar).
   Embodiment 15. The compound of embodiment 8 or 9, wherein T comprises an imaging agent, wherein the imaging agent comprises a radionuclide or a fluorescent dye.
   Embodiment 16. The compound of embodiment 8 or 9, wherein T comprises a drug, wherein the drug comprises a chelating moiety chelated to a radionuclide.
   Embodiment 17. The compound of embodiment 1, wherein the compound is of Formula Ia

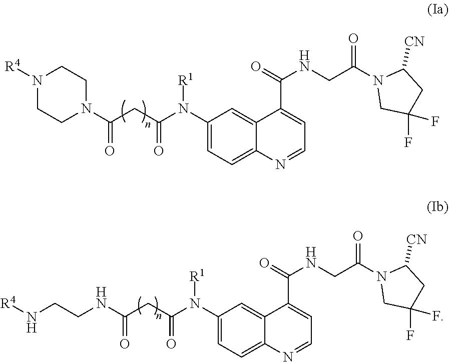

Embodiment 18. The compound of embodiment 17, wherein R¹ is H or methyl.
Embodiment 19. The compound of embodiment 17 or 18, wherein R⁴ is -L-T.
Embodiment 20. The compound of any one of embodiments 17-19, wherein: T comprises a chelating moiety chelated to a radionuclide, wherein the chelating moiety is selected from DOTAGA, DOTA, NOTA, NODAGA, and NODA; and the radionuclide is selected from ⁶¹Cu, ⁶⁴Cu, and ⁶⁷Cu.
Embodiment 21. The compound of embodiment 1, wherein the compound is selected from:

| Compound | Structure | |
|---|---|---|
| 1 | 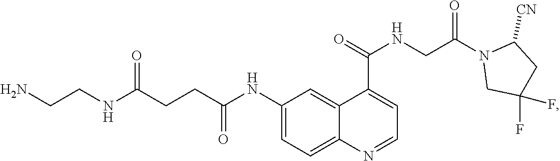 [SMILES: N#C[C@@H]1CC(F)(F)CN1C(=O)CNC(=O)c1ccnc2ccc(NC(=O)CCC(=O)NCCN)cc12] | |
| 2 | 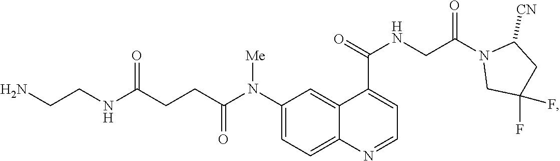 [SMILES: CN(C(=O)CCC(=O)NCCN)c1ccc2nccc(C(=O)NCC(=O)N3CC(F)(F)C[C@H]3C#N)c2c1] | |
| 3 | 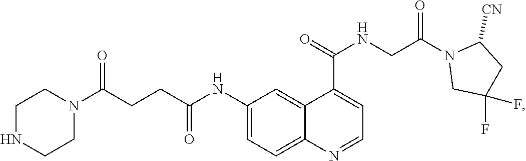 [SMILES: N#C[C@@H]1CC(F)(F)CN1C(=O)CNC(=O)c1ccnc2ccc(NC(=O)CCC(=O)N3CCNCC3)cc12] | |
| 4 | 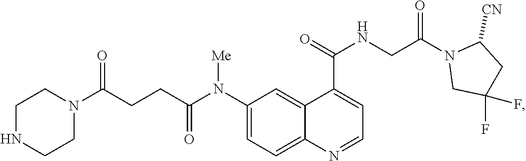 [SMILES: CN(C(=O)CCC(=O)N1CCNCC1)c1ccc2nccc(C(=O)NCC(=O)N3CC(F)(F)C[C@H]3C#N)c2c1] | |
| NODAGA-1 | 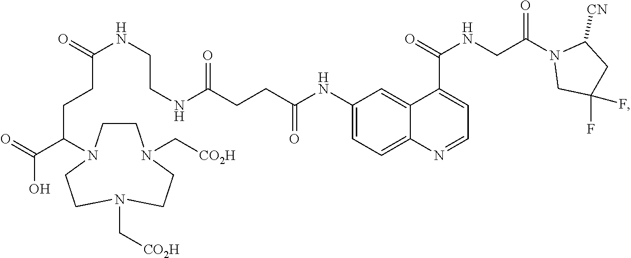 [SMILES: N#C[C@@H]1CC(F)(F)CN1C(=O)CNC(=O)c1ccnc2ccc(NC(=O)CCC(=O)NCCNC(=O)CCC(C(=O)O)N3CCN(CC(=O)O)CCN(CC(=O)O)CC3)cc12] | |
| NODAGA-2 | 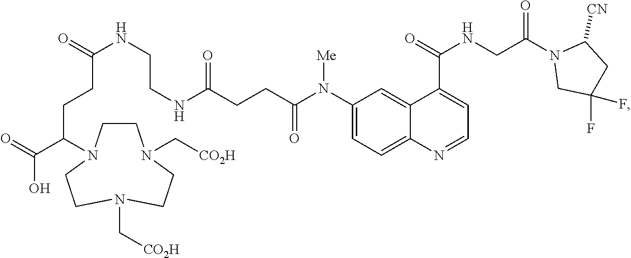 [SMILES: CN(C(=O)CCC(=O)NCCNC(=O)CCC(C(=O)O)N1CCN(CC(=O)O)CCN(CC(=O)O)CC1)c1ccc2nccc(C(=O)NCC(=O)N3CC(F)(F)C[C@H]3C#N)c2c1] | |
| NODAGA-3 | 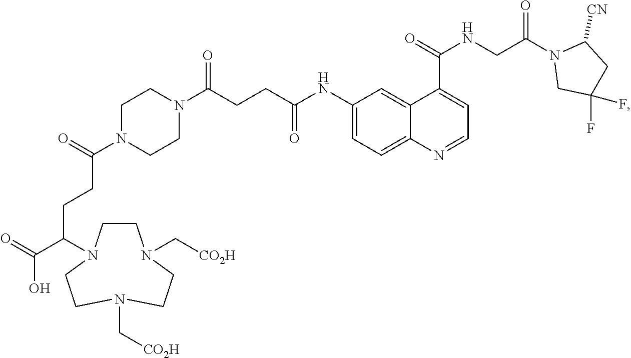 [SMILES: N#C[C@@H]1CC(F)(F)CN1C(=O)CNC(=O)c1ccnc2ccc(NC(=O)CCC(=O)N3CCN(C(=O)CCC(C(=O)O)N4CCN(CC(=O)O)CCN(CC(=O)O)CC4)CC3)cc12] | |
| NODAGA-4 | 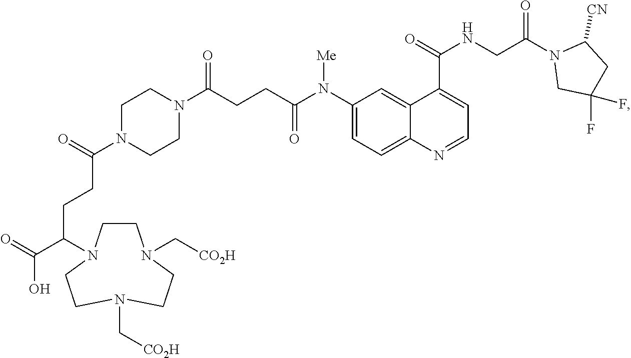 [SMILES: CN(C(=O)CCC(=O)N1CCN(C(=O)CCC(C(=O)O)N2CCN(CC(=O)O)CCN(CC(=O)O)CC2)CC1)c1ccc2nccc(C(=O)NCC(=O)N3CC(F)(F)C[C@H]3C#N)c2c1] | |
| *Cu-NODAGA-1 | 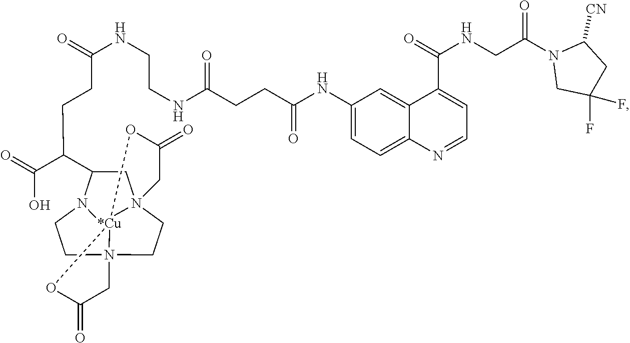 | |
| *Cu-NODAGA-2 | 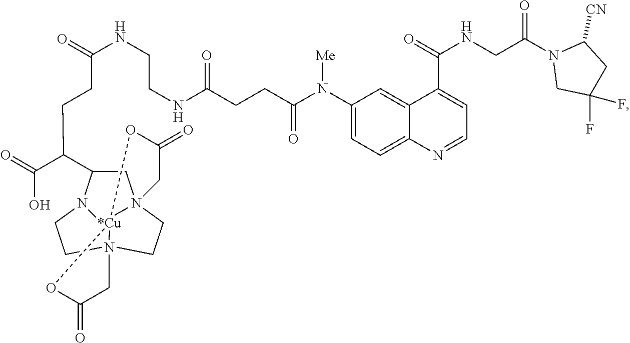 | |
| *Cu-NODAGA-3 | 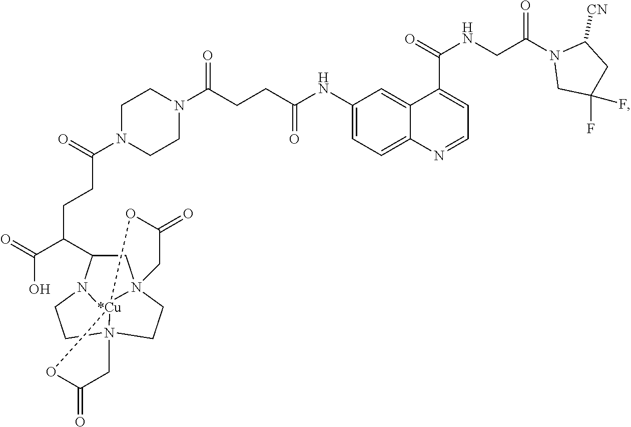 | and |

| | |
|---|---|
| *Cu-NODAGA-4 | 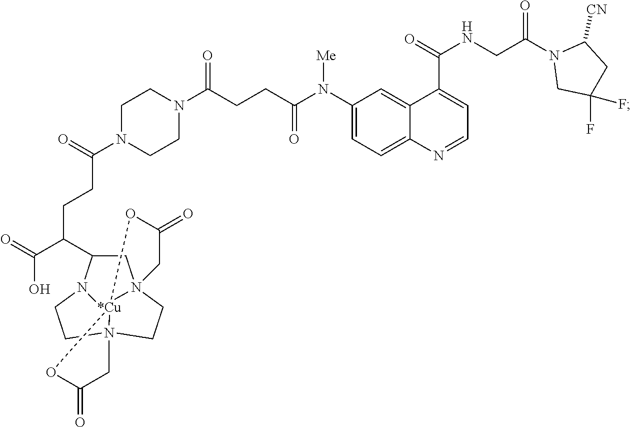 |

or is a pharmaceutically acceptable salt thereof, wherein *Cu is selected from ⁶¹Cu, ⁶²Cu, ⁶⁴Cu, and ⁶⁷Cu, particularly ⁶¹Cu and ⁶⁷Cu.
   Embodiment 22. A pharmaceutical composition comprising a compound of any one of embodiments 1-21 and a pharmaceutically acceptable excipient.
   Embodiment 23. The pharmaceutical composition of embodiment 22, wherein R⁴ is -L-T, the composition is characterized by one or more of: (i) molar activity of ≥3 MBq/nmol, (ii) radiochemical purity≥91%, (iii) activity concentration of ≥8 MBq/mL, and (iv) by radionuclidic purity of the compound at end of synthesis (EoB plus 2 hours) of 95%.
   Embodiment 24. A method of generating one or more images of a subject comprising:
administering to a subject an effective amount of a compound of embodiment 13, wherein the radionuclide is ⁶¹Cu; and
generating one or more images of at least a part of the subject's body.
   Embodiment 25. The method of embodiment 24, wherein the image is generated using positron emission tomography (PET), PET-computer tomography (PET-CT), or single-photon emission computerized tomography (SPECT).
   Embodiment 26. A method of treating a disease in a patient in need thereof, comprising administering to the patient an effective amount of a compound according to any one of embodiments 1-14, or 16-20.
   Embodiment 27. The method of embodiment 26, wherein the disease is selected from cancers, inflammatory diseases, infectious diseases, and immune diseases.
   Embodiment 28. A theranostic method comprising:
(a) administering to a subject an effective amount of a first compound of embodiment 13, wherein the radionuclide is ⁶¹Cu, or a pharmaceutical composition comprising an effective amount of a first compound of embodiment 13, wherein the radionuclide is ⁶¹Cu;
(b) generating one or more images of the subject; and
(c) administering to the subject an effective amount of a second compound of embodiment 13, wherein the radionuclide is ⁶⁷Cu, or a pharmaceutical composition comprising an effective amount of a second compound of embodiment 13, wherein the radionuclide is ⁶⁷Cu, wherein the first and second compounds of steps (a) and (c) differ only in radioisotopic identity.
   Embodiment 29. The method of embodiment 28, wherein:
(a) the first compound is ⁶¹[Cu]Cu-NODAGA-1 and the second compound is ⁶⁷[Cu]Cu-NODAGA-1;
(b) the first compound is ⁶¹[Cu]Cu-NODAGA-2 and the second compound is ⁶⁷[Cu]Cu-NODAGA-2;
(c) the first compound is ⁶¹[Cu]Cu-NODAGA-3 and the second compound is ⁶⁷[Cu]Cu-NODAGA-3; or
(d) the first compound is ⁶¹[Cu]Cu-NODAGA-4 and the second compound is ⁶⁷[Cu]Cu-NODAGA-4.
   Embodiment 30. The method of embodiment 28 or 29, further comprising determining, via the one or more images of the subject, the presence or absence of a disease in the subject based on the presence or absence of localization of the ⁶¹Cu radionuclide of the first compound in the subject's body.

### 6. EXAMPLES

### Example 1: Synthesis of FAP Inhibitors

### 1.1: Synthesis of (S)—N1-(2-aminoethyl)-N4-(4-((2-(2-cyano-4,4-difluoropyrrolidin-1-yl)-2-oxoethyl)carbamoyl)quinolin-6-yl)succinimide (1)

### Step 1: (S)-6-amino-N-(2-(2-cyano-4,4-difluoropyrrolidin-1-yl)-2-oxoethyl)quinoline-4-carboxamide (A)

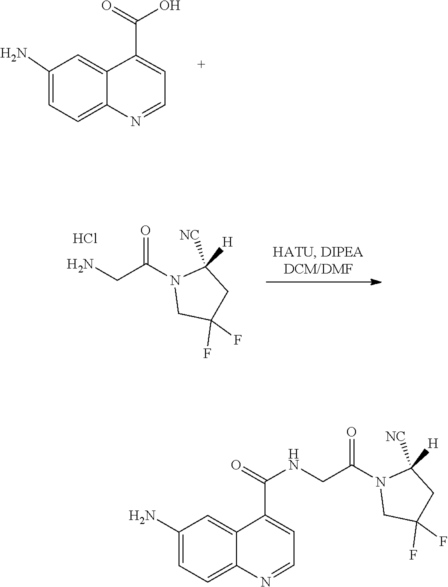 [SMILES: Nc1ccc2nccc(C(=O)O)c2c1 ; Cl ; N#C[C@@H]1CC(F)(F)CN1C(=O)CN ; N#C[C@@H]1CC(F)(F)CN1C(=O)CNC(=O)c1ccnc2ccc(N)cc12]

The two precursors (purchased from AstaTech) were dissolved together with HATU in DMF and then DCM was added. DIPEA was added dropwise and the reaction was monitored via LC/MS. The reaction was complete after less than 1 h. The crude product was concentrated, diluted with Water/ACN 85:15 and directly purified via HPLC (LCMS-2020 Shimadzu system equipped with a Gemini C-6 Phenyl column (10×250 mm, 5 μm particle size). The gradient used was 5-80% solvent B in 15 min (A=H₂O [0.1% TFA], B=ACN [0.1% TFA]) at a flow rate of 5.0 mL/min) to provide A as a pure red powder (38 mg, 84% yield).

### Step 2: Synthesis of (S)-4-((4-((2-(2-cyano-4,4-difluoropyrrolidin-1-yl)-2-oxoethyl)carbamoyl)quinolin-6-yl)amino)-4-oxobutanoic Acid (B)

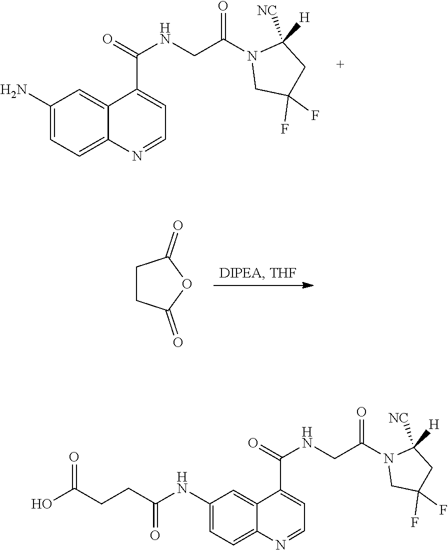 [SMILES: N#C[C@@H]1CC(F)(F)CN1C(=O)CNC(=O)c1ccnc2ccc(N)cc12 ; O=C1CCC(=O)O1 ; N#C[C@@H]1CC(F)(F)CN1C(=O)CNC(=O)c1ccnc2ccc(NC(=O)CCC(=O)O)cc12]

(S)-6-amino-N-(2-(2-cyano-4,4-difluoropyrrolidin-1-yl)-2-oxoethyl)quinoline-4-carboxamide (A) and succinic anhydride were dissolved in THF. DIPEA was added dropwise and the reaction was mixed overnight and checked via LC/MS. The crude product was directly purified via HPLC (LCMS-2020 Shimadzu system equipped with a Gemini C-6 Phenyl column (10×250 mm, 5 μm particle size). The gradient used was 5-80% solvent B in 8 min (A=H₂O [0.1% TFA], B=ACN [0.1% TFA]) at a flow rate of 5.0 mL/min) to afford B as a yellow powder (32.7 mg, 68% yield).

### Step 3: (S)—N1-(2-aminoethyl)-N4-(4-((2-(2-cyano-4,4-difluoropyrrolidin-1-yl)-2-oxoethyl)carbamoyl)quinolin-6-yl)succinimide (1)

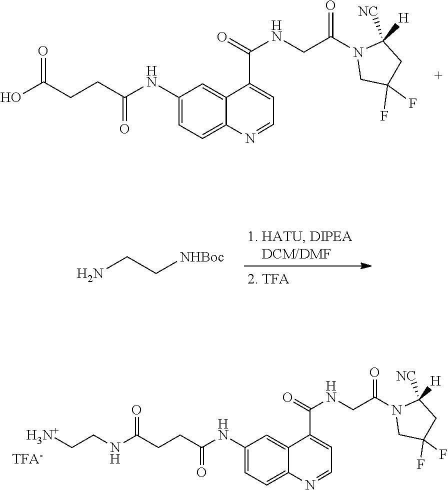 [SMILES: N#C[C@@H]1CC(F)(F)CN1C(=O)CNC(=O)c1ccnc2ccc(NC(=O)CCC(=O)O)cc12 ; CC(C)(C)OC(=O)NCCN ; N#C[C@@H]1CC(F)(F)CN1C(=O)CNC(=O)c1ccnc2ccc(NC(=O)CCC(=O)NCC[NH3+])cc12]

S)-4-((4-((2-(2-cyano-4,4-difluoropyrrolidin-1-yl)-2-oxoethyl)carbamoyl)quinolin-6-yl)amino)-4-oxobutanoic acid (B), HATU and the amine were dissolved in DCM and DMF. DIPEA was added dropwise and the reaction was mixed and checked via LC/MS. After completion, TIPS was added and TFA was added dropwise: first, the DIPEA was quenched. The deprotection step took over in 2 days. The crude material was used without further purification.

### 1.2: Synthesis of (S)—N1-(2-aminoethyl)-N4-(4-((2-(2-cyano-4,4-difluoropyrrolidin-1-yl)-2-oxoethyl)carbamoyl)quinolin-6-yl)-N4-methylsuccinamide (2)

Compound 2 was prepared as shown in Scheme 1:

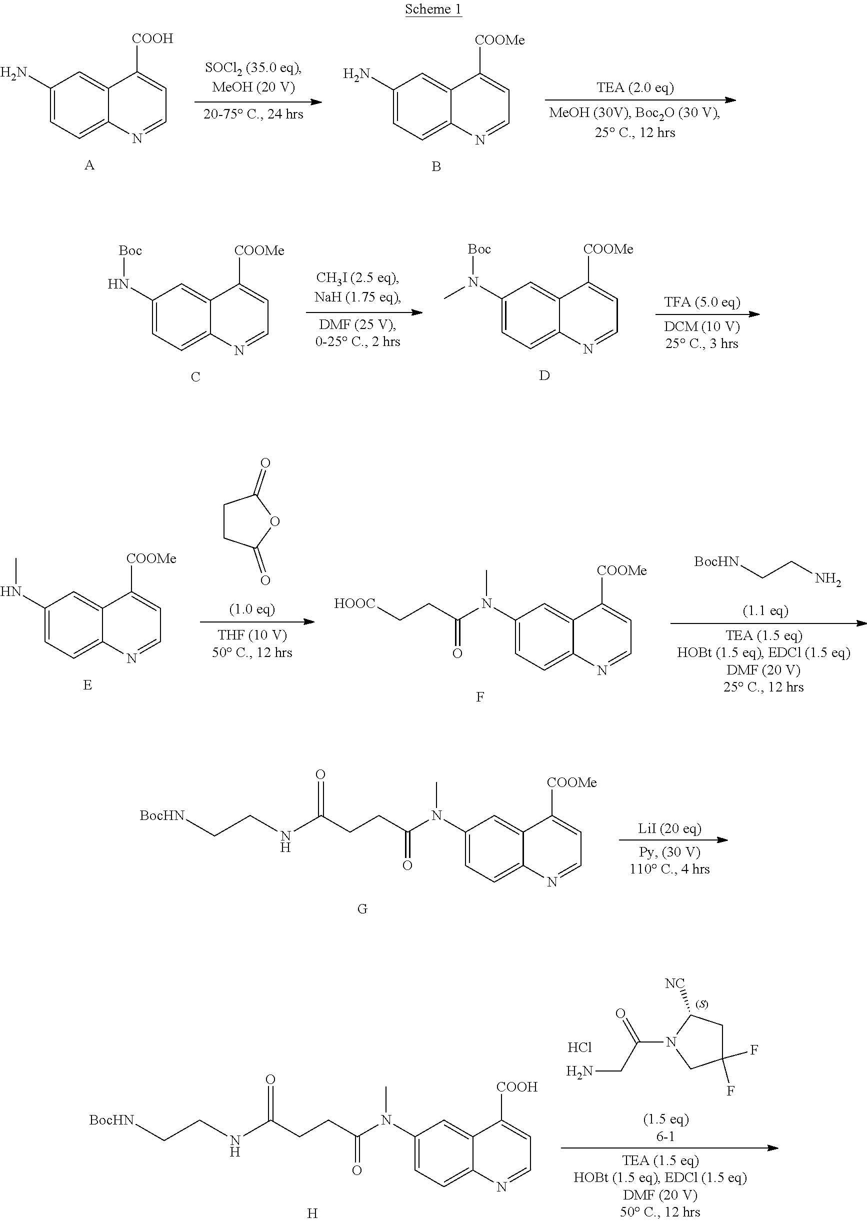

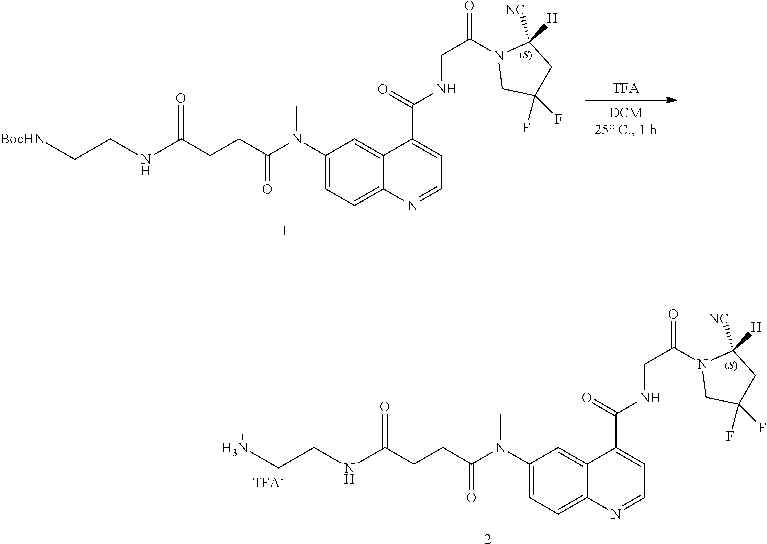

Step 1: To a mixture of compound A (4.17 g, 22.2 mmol) in MeOH (84.0 mL) was added SOCl₂ (26.4 g, 222 mmol, 16.1 mL) in one portion at 0-5° C. under N₂. The reaction was stirred at 0-5° C. for 0.5 h. The mixture was heated to 75° C. and stirred for 12 hrs. The mixture was added SOCl₂ (26.4 g, 222 mmol, 16.1 mL) and stirred for 12 hrs at 75° C. The mixture was added SOCl₂ (26.4 g, 222 mmol, 16.1 mL) and stirred for 12 hrs at 75° C. The mixture was added SOCl₂ (13.2 g, 111 mmol, 8.04 mL) and stirred for 12 hrs at 75° C. LC-MS showed one main peak with desired mass was detected. The mixture was concentrated in vacuum. The crude product was triturated with MeCN (300 mL) at 20° C. for 1 hr to afford compound B (7.05 g, crude) as a brown solid. ¹H NMR: (400 MHz, DMSO-d₆) δ 8.81 (d, J=4.8 Hz, 1H), 8.27 (d, J=8.8 Hz, 1H), 8.10 (d, J=4.8 Hz, 1H), 7.82 (s, 1H), 7.67 (d, J=8.0 Hz, 1H), 3.98 (s, 3H). LC-MS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, product: RT=1.262 min).

Step 2: To a solution of B (7.02 g, 34.7 mmol) in MeOH (100 mL), Boc₂O (100 mL) was added TEA (7.03 g, 69.4 mmol), the mixture was stirred at 25° C. for 12 hrs. LCMS showed compound B consumed and one peak of desired MS was detected. The mixture was concentrated in vacuum. The residue was purified by column chromatography (SiO₂, Petroleum ether/Ethyl acetate=100/1 to 1/1, compound C Rf=0.35) to obtain compound C (4.36 g, 41.5% yield) as a brown solid. ¹H NMR: (400 MHz, CDCl₃) δ 8.89 (d, J=4.4 Hz, 1H), 8.78 (d, J=2.4 Hz, 1H), 8.11 (d, J=9.2 Hz, 1H), 7.96-7.89 (m, 2H), 6.83 (s, 1H), 4.04 (s, 3H), 1.57 (s, 9H).

Step 3: To a solution of compound C (3.36 g, 11.1 mmol) in DMF (84.0 mL) was added NaH (778 mg, 19.5 mmol, 60% purity) in portions at 0° C., the mixture was stirred at 25° C. for 20 mins. Mel (3.94 g, 27.8 mmol) was added to the reaction mixture at 25° C. and stirred at 25° C. for 2 hrs. LCMS (ET60385-17-P1A3, Product RT=0.562 min) showed compound C consumed and one peak of desired MS was detected. The reaction mixture was cooled to 0° C. and quenched with brine (80.0 mL), extracted with EtOAc (3×100 mL). The organic layer was dried over sodium sulfate, filtered and concentrated in vacuum to obtain compound D (4.78 g, crude) as a brown solid.

Step 4: To a solution of compound D (4.78 g, 15.1 mmol) in DCM (50.0 mL) was added dropwise TFA (8.61 g, 75.5 mmol), the mixture was stirred at 25° C. for 12 hrs. LCMS showed compound D consumed and one peak of desired MS was detected. The reaction mixture was quenched with saturated NaHCO₃ (50.0 mL), extracted with DCM (3×40.0 mL). The organic layer was dried over sodium sulfate, filtered and concentrated in vacuum. The residue was purified by column chromatography (SiO₂, Petroleum ether/Ethyl acetate=100/1 to 1/1, product Rf=0.40) to obtain compound E (2.51 g, 76.8% yield) as a brown solid. ¹H NMR: ET60385-19-P1A1 (400 MHz, CDCl₃) δ 8.67 (d, J=4.4 Hz, 1H), 7.94 (d, J=9.2 Hz, 1H), 7.85 (d, J=4.4 Hz, 1H), 7.80 (d, J=2.4 Hz, 1H), 7.17-7.14 (m, 1H), 4.02 (s, 3H), 3.01 (s, 3H).

Step 5: To a solution of compound E (500 mg, 2.31 mmol) in THE (4.00 mL) was added tetrahydrofuran-2,5-dione (231 mg, 2.31 mmol), the reaction mixture was stirred at 50° C. for 12 hrs. LCMS showed compound E consumed and one peak of desired MS was detected. The mixture was concentrated in vacuum to obtain compound F (716 mg, crude) as a brown solid. ¹H NMR: ET60385-43-P1A1 (400 MHz, CDCl₃) δ 9.10 (d, J=4.0 Hz, 1H), 8.77 (d, J=2.4 Hz, 1H), 8.28 (d, J=8.8 Hz, 1H), 8.03 (d, J=4.0 Hz, 1H), 7.66-7.64 (m, 1H), 4.06 (s, 3H), 3.42 (s, 3H), 2.69-2.66 (m, 2H), 2.51-2.50 (m, 2H).

Step 6: To a solution of compound F (716 mg, 2.26 mmol) in DMF (7.00 mL) was added TEA (343 mg, 3.40 mmol), HOBt (458 mg, 3.40 mmol), EDCI (650 mg, 3.40 mmol) and tert-butyl N-(2-aminoethyl)carbamate (398 mg, 2.49 mmol), the reaction mixture was stirred at 25° C. for 12 hrs. LCMS showed compound F consumed and one peak of desired MS was detected. The reaction mixture was quenched with saturated NaHCO₃ (15.0 mL), extracted with DCM (25.0 mL×3) washed with brine (15.0 mL). The organic layer was dried over sodium sulfate, filtered and concentrated in vacuum to obtain compound G (1.33 g, crude) as a brown solid.

Step 7: To a solution of compound G (1.33 g, 2.90 mmol) in Py. (20.0 mL) was added LiI (7.86 g, 58.6 mmol), the mixture was stirred at 110° C. for 4 hrs. LCMS showed compound G consumed and one peak of desired MS was detected. The mixture was concentrated in vacuum. The residue was purified by prep-HPLC (column: Welch Xtimate C18 250*100 mm #10 um; mobile phase: [water (NH₄HCO₃)-ACN]; B %: 1%-30%, 20 min) to obtain compound H (647 mg, 50.1% yield) as an off-white solid.

Step 8: To a solution of compound H (617 mg, 1.39 mmol) in DMF (6.00 mL) was added DIEA (717 mg, 5.55 mmol), HATU (791 mg, 2.08 mmol) and compound 6-1 (587 mg, 2.08 mmol, 80% purity, HCl), the mixture was stirred at 25° C. for 1 hr. LCMS showed compound H consumed and one peak of desired MS was detected. The reaction mixture was quenched with saturated NaHCO₃ (15.0 mL), extracted with DCM (25.0 mL×3) washed with brine (15.0 mL). The organic layer was dried over sodium sulfate, filtered and concentrated in vacuum to obtain compound I (2.70 g, crude) as a brown solid.

Step 9: To a solution of compound I (2.70 g, 4.39 mmol) in DCM (10.0 mL) was added TFA (41.5 g, 364 mmol), the mixture was stirred at 25° C. for 1 hr. LCMS (ET60385-61-P1A4, Product RT=0.490 min) showed compound I consumed and one peak of desired MS was detected. The mixture was concentrated in vacuum. The residue was purified by prep-HPLC (column: Welch Xtimate C18 250*100 mm #10 um; mobile phase: [water (NH₄HCO₃)-ACN]; B %: 5%-35%, 20 min) to obtain compound 2 (260 mg, 11.1% yield, 97.3% purity) as a brown solid. LCMS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, Product RT=0.493 min).

### 1.3: Synthesis of (S)—N-(2-(2-cyano-4,4-difluoropyrrolidin-1-yl)-2-oxoethyl)-6-(4-oxo-4-(piperazin-1-yl)butanamido)quinoline-4-carboxamide (3)

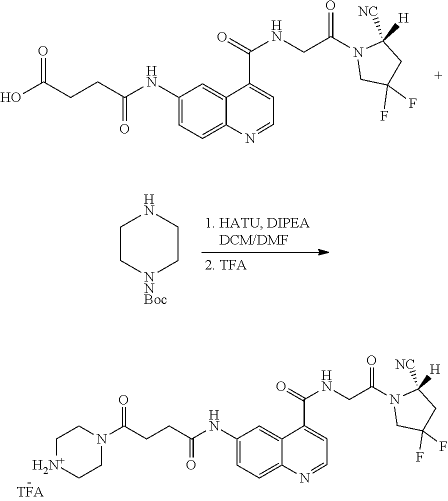

(S)-4-((4-((2-(2-cyano-4,4-difluoropyrrolidin-1-yl)-2-oxoethyl)carbamoyl)quinolin-6-yl)amino)-4-oxobutanoic acid, HATU and the amine were dissolved in DCM and DMF. DIPEA was added dropwise and the reaction was checked. When all the coupling occurred, the crude product was concentrated a bit and then TIPS was added. TFA was added dropwise and the mixture was checked via LC/MC until completion. Crude product (3) was used as such.

### 1.4: Synthesis of (S)—N-(2-(2-cyano-4,4-difluoropyrrolidin-1-yl)-2-oxoethyl)-6-(N-methyl-4-oxo-4-(piperazin-1-yl)butanamido)quinoline-4-carboxamide (4)

Compound 4 was prepared as shown in Scheme 2:

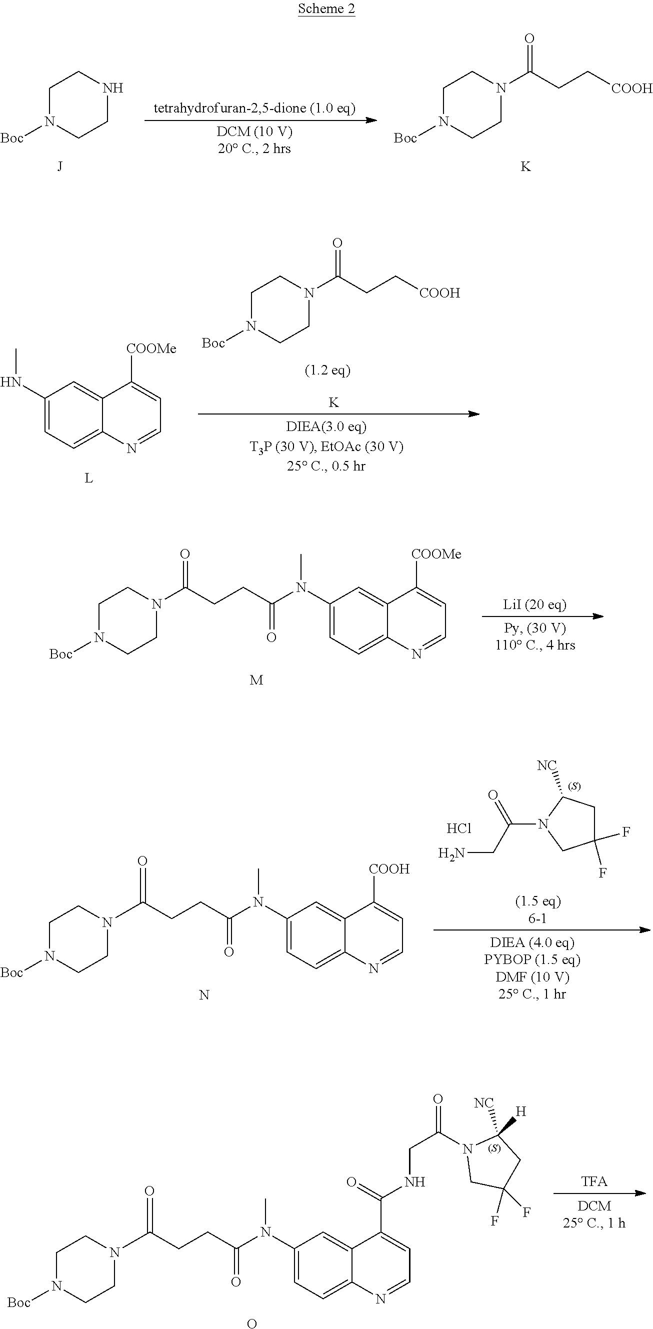

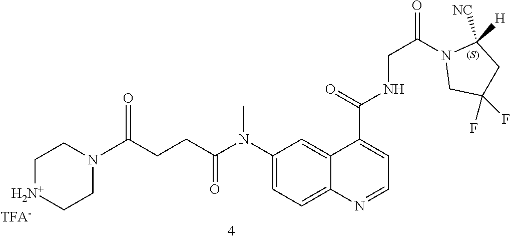

Step 1: To a mixture of compound J (10.0 g, 53.7 mmol) in DCM (70.0 mL) was added tetrahydrofuran-2, 5-dione (5.37 g, 53.7 mmol). The mixture was stirred for 2 hrs at 20° C. TLC (dichloromethane/methanol/AcOH=9/1/0.01, compound J Rf=0.0) showed the reaction was completed. The mixture was concentrated in vacuum. The residue was purified by silica gel chromatography (dichloromethane/methanol=100/1, 9/1) to afford compound K (4.75 g, 30.9% yield) as a white solid. ¹H NMR: (400 MHz, CDCl₃) δ 10.56-11.09 (m, 1H), 3.53-3.62 (m, 2H), 3.45 (s, 4H), 3.36-3.42 (m, 2H), 2.60-2.73 (m, 4H), 1.45 (s, 9H).

Step 2: To a solution of compound L (300 mg, 1.39 mmol) in EtOAc (10.0 mL) was added DIEA (537 mg, 4.16 mmol), compound K (476 mg, 1.66 mmol) and T₃P (11.2 g, 17.7 mmol, 50% purity), the reaction mixture was stirred at 25° C. for 0.5 hr. LCMS showed compound L consumed and one peak of desired MS was detected. Then reaction mixture is diluted with EtOAc (20.0 mL), washed with water (60.0 mL), saturated NaHCO₃ (60.0 mL), and brine (20.0 mL). The organic phase is dried over Na₂SO₄ and concentrated in vacuum to obtain compound M (716 mg, crude) as brown oil.

Step 3: To a solution of compound M (716 mg, 1.48 mmol) in Py. (20.0 mL) was added LiI (3.96 g, 29.5 mmol), the mixture was stirred at 110° C. for 4 hrs. LCMS showed compound M consumed and one peak of desired MS was detected. The mixture was concentrated in vacuum. The residue was purified by prep-HPLC (column: Welch Xtimate C18 250*100 mm #10 um; mobile phase: [water (NH₄HCO₃)-ACN]; B %: 1%-30%, 20 min) to obtain compound N (460 mg, 64.4% yield, 97.4% purity) as an off-white solid. LCMS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, Product RT=0.596 min)

Step 4: To a solution of compound N (460 mg, 977 umol) in DMF (5.00 mL) was added DIEA (505 mg, 3.91 mmol), PYBOP (763 mg, 1.47 mmol) and compound 6-1 (330 mg, 1.47 mmol, HCl), the mixture was stirred at 25° C. for 1 hr. LCMS showed one peak of desired MS was detected. The reaction mixture was quenched with saturated NaHCO₃ (15.0 mL), extracted with DCM (25.0 mL×3) washed with brine (15.0 mL). The organic layer was dried over Na₂SO₄, filtered and concentrated in vacuum to obtain compound O (2.10 g, crude) as brown oil.

Step 5: To a solution of compound O (2.10 g, 3.27 mmol) in DCM (10.0 mL) was added TFA (15.4 g, 135 mmol), the mixture was stirred at 25° C. for 1 hr. LCMS showed compound O consumed and one peak of desired MS was detected. The mixture was concentrated in vacuum. The residue was purified by prep-HPLC (column: Welch Xtimate C18 250*70 mm #10 um; mobile phase: [water (NH₄HCO₃)-ACN]; B %: 0%-40%, 20 min) to obtain compound 4 (196 mg, 11.0% yield) as an off-white solid.

### 1.5: Synthesis of FAPI-46

FAPI-46 was prepared as shown in Scheme 3:

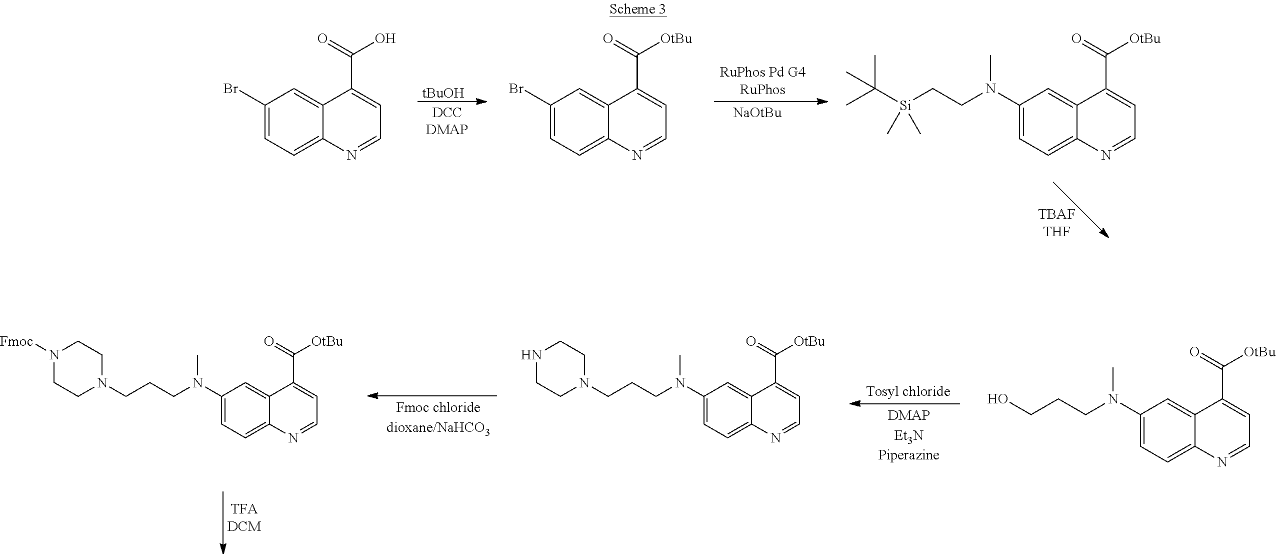

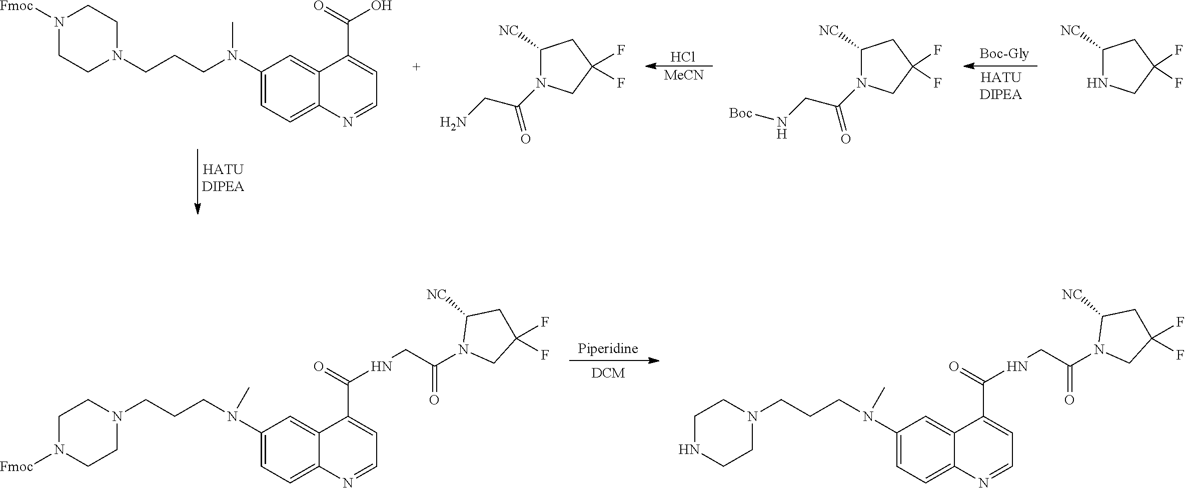

FAPI-46 can also be prepared according to the method described in WO 2019/154886A1.

### Example 2: Synthesis of FAPI-NODAGA Conjugates

### 2.1: Synthesis of 2,2′-(7-((R)-1-carboxy-4-((2-(4-((4-((2-((S)-2-cyano-4,4-difluoropyrrolidin-1-yl)-2-oxoethyl)carbamoyl)quinolin-6-yl)amino)-4-oxobutanamido)ethyl)amino)-4-oxobutyl)-1,4,7-trIazonane-1,4-diyl)dIacetic Acid ((R)-NODAGA-1)

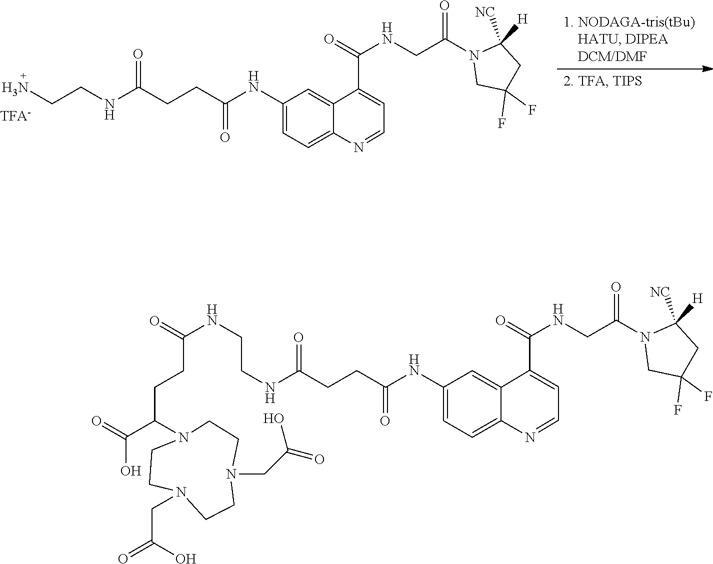 [SMILES: N#C[C@@H]1CC(F)(F)CN1C(=O)CNC(=O)c1ccnc2ccc(NC(=O)CCC(=O)NCCN)cc12 ; N#C[C@@H]1CC(F)(F)CN1C(=O)CNC(=O)c1ccnc2ccc(NC(=O)CCC(=O)NCCNC(=O)CCC(C(=O)O)N3CCN(CC(=O)O)CCN(CC(=O)O)CC3)cc12]

To the (S)—N1-(2-aminoethyl)-N4-(4-((2-(2-cyano-4,4-difluoropyrrolidin-1-yl)-2-oxoethyl)carbamoyl)quinolin-6-yl)succinimide (1) crude solution, DIPEA was added dropwise to neutralize TFA. Then, HATU and NODAGA-Tris(tBu) were added dropwise as DMSO solution (150 μL). The reaction was complete after a few minutes. The crude product was concentrated and purified via HPLC. To the pure material, DCM, TIPS and TFA were added and the reaction was left for 1 day until completion and purified via HPLC to obtain 15.8 mg of (R)-NODAGA-1 as a pale yellow powder (Yield: 51%).

### 2.2: Synthesis of 2,2′-(7-((R)-1-carboxy-4-((2-(4-((4-((2-((S)-2-cyano-4,4-difluoropyrrolidin-1-yl)-2-oxoethyl)carbamoyl)quinolin-6-yl)(methyl)amino)-4-oxobutanamido)ethyl)amino)-4-oxobutyl)-1,4,7-triazonane-1,4-diyl)diacetic acid ((R)-NODAGA-2)

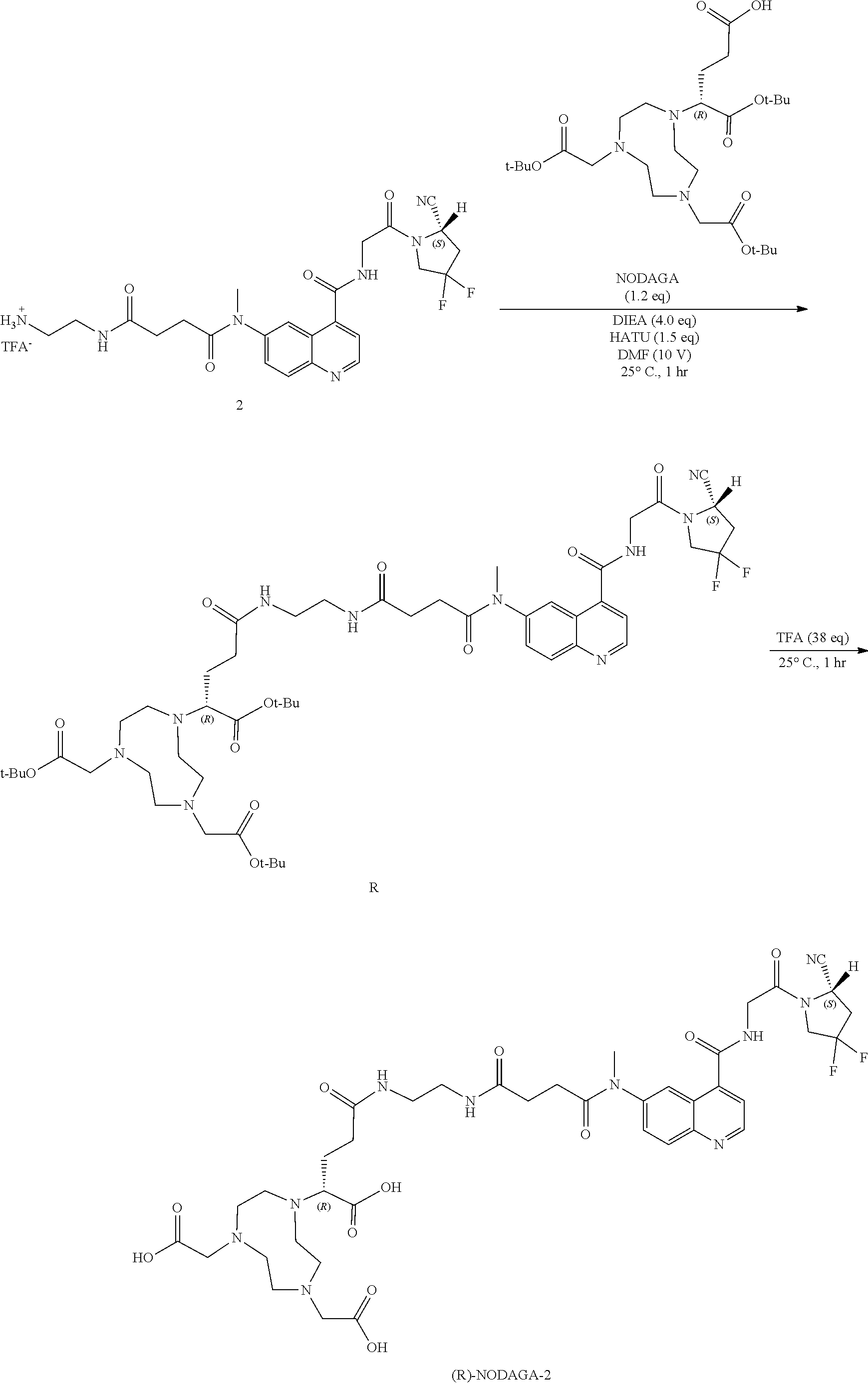

Step 1: To a solution of compound 2 (80.0 mg, 155 μmol) in DMF (1.00 mL) was added DIEA (80.2 mg, 620 μmol), HATU (121 mg, 232 μmol) and NODAGA-Tris(tBu) (101 mg, 186 μmol), the mixture was stirred at 25° C. for 1 hr. LCMS showed compound 2 consumed and one peak of desired MS was detected. The reaction mixture was quenched with saturated NaHCO₃ (4.00 mL), extracted with DCM (10.0 mL×3) washed with brine (10.0 mL). The organic layer was dried over sodium sulfate, filtered, and concentrated in vacuum to obtain R (310 mg, crude) was obtained as brown oil.

Step 2: To a solution of compound R (310 mg, 297 μmol) in TFA (1.29 g, 11.3 mmol) at 25° C., the mixture was stirred at 25° C. for 1 hr. LCMS showed compound R consumed and one peak of desired MS was detected. The mixture was concentrated in vacuum. The crude product on notebook page ET60385-73 (220 mg, crude) and ET60385-78 (206 mg, crude) was combined for further purification. The residue was purified by prep-HPLC (column: C18-1 150*30 mm*5 um; mobile phase:[water (TFA)-ACN]; B %: 5%-35%, 20 min) to obtained (R)-NODAGA-2 (10.01 mg, 3.30% yield, 96.9% purity, TFA) a brown solid. ¹H NMR: ET60385-73-P1A2 (400 MHz, D₂O) δ 9.14 (d, J=5.2 Hz, 1H), 8.32-9.30 (m, 2H), 8.02-7.98 (m, 2H), 5.18-5.14 (m, 1H), 4.38 (s, 2H), 4.33-4.24 (m, 1H), 4.20-4.10 (m, 1H), 3.76 (s, 4H), 3.51-3.31 (m, 4H), 3.25-3.12 (m, 12H), 3.03-2.87 (m, 6H), 2.49 (s, 3H), 2.30 (t, J=7.2 Hz, 2H), 2.03-1.85 (m, 1H). LCMS (ET60385-73-P1Z1, Product RT=1.610 min).

### 2.3: Synthesis of 2,2′-(7-((R)-1-carboxy-4-(4-(4-((4-((2-((S)-2-cyano-4,4-difluoropyrrolidin-1-yl)-2-oxoethyl)carbamoyl)quinolin-6-yl)amino)-4-oxobutanoyl)piperazin-1-yl)-4-oxobutyl)-1,4,7-triazonane-1,4-diyl)diacetic acid ((R)-NODAGA-3)

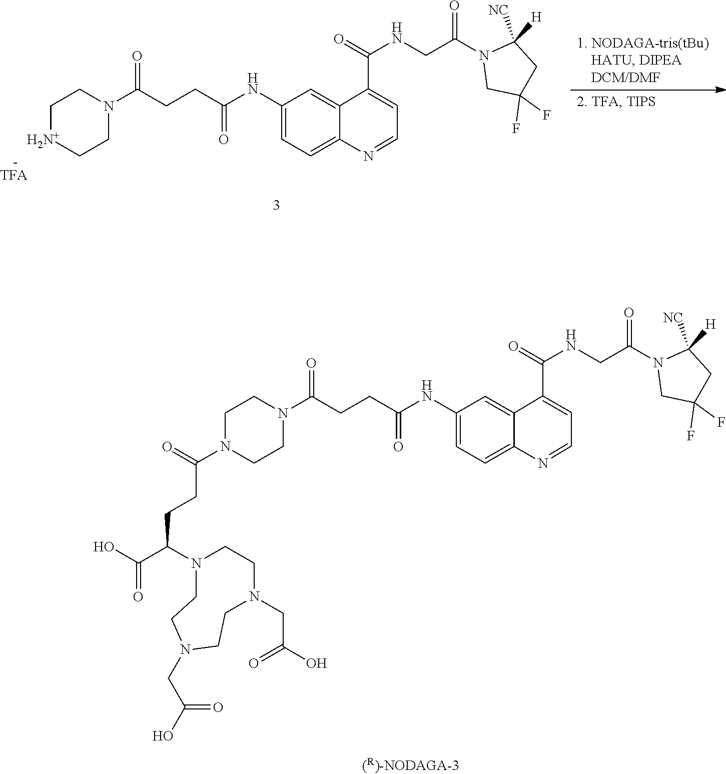 [SMILES: N#C[C@@H]1CC(F)(F)CN1C(=O)CNC(=O)c1ccnc2ccc(NC(=O)CCC(=O)N3CC[NH2+]CC3)cc12 ; N#C[C@@H]1CC(F)(F)CN1C(=O)CNC(=O)c1ccnc2ccc(NC(=O)CCC(=O)N3CCN(C(=O)CC[C@H](C(=O)O)N4CCN(CC(=O)O)CCN(CC(=O)O)CC4)CC3)cc12]

To the (S)—N-(2-(2-cyano-4,4-difluoropyrrolidin-1-yl)-2-oxoethyl)-6-(4-oxo-4-(piperazin-1-yl)butanamido)quinoline-4-carboxamide crude solution, DIPEA was added dropwise to neutralize TFA. Then, HaTU and NODAGA-Tris(TBu) were added dropwise as DMSO solution (150 μL). The reaction was complete after a few minutes. The crude product was concentrated and purified via HPLC. To the pure material, DCM, TIPS and TFA were added and the reaction was left for 1 day until completion and purified via HPLC to obtain 15.8 mg of (R)-NODAGA-3 as a pale yellow powder (Yield: 26%).

### 2.4: Synthesis of 2,2′-(7-((R)-1-carboxy-4-(4-(4-((4-((2-((S)-2-cyano-4,4-difluoropyrrolidin-1-yl)-2-oxoethyl)carbamoyl)quinolin-6-yl)(methyl)amino)-4-oxobutanoyl)piperazin-1-yl)-4-oxobutyl)-1,4,7-triazonane-1,4-diyl)diacetic acid ((R)-NODAGA-4)

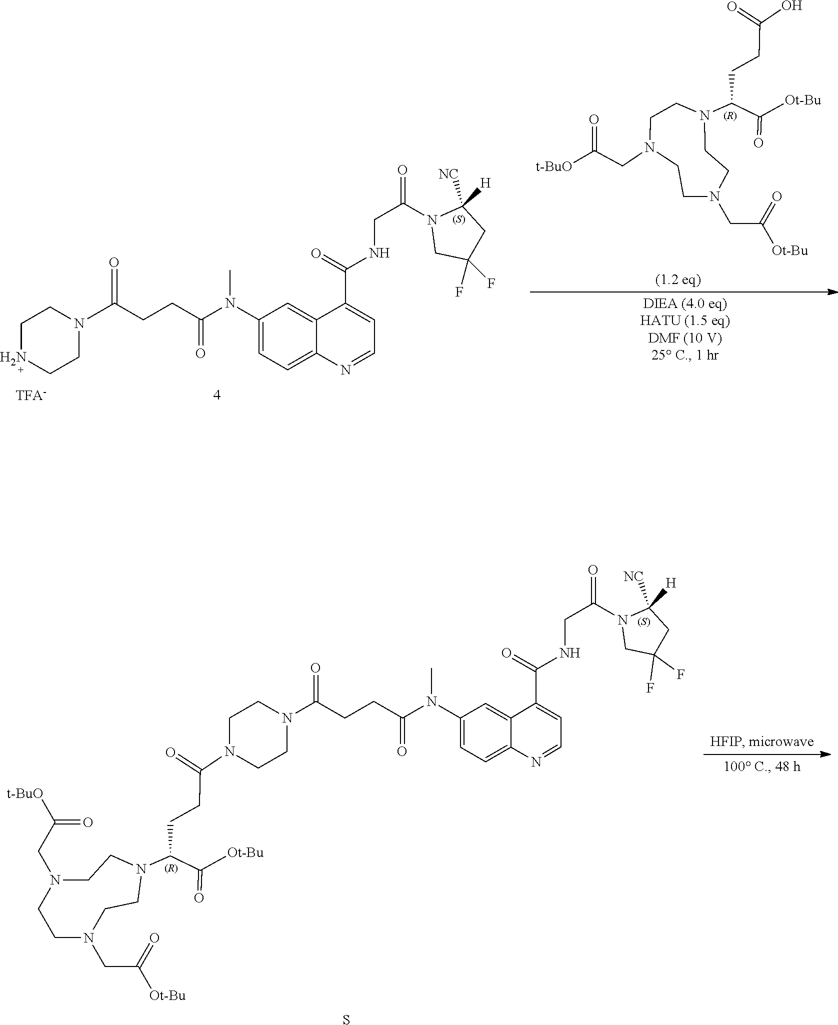

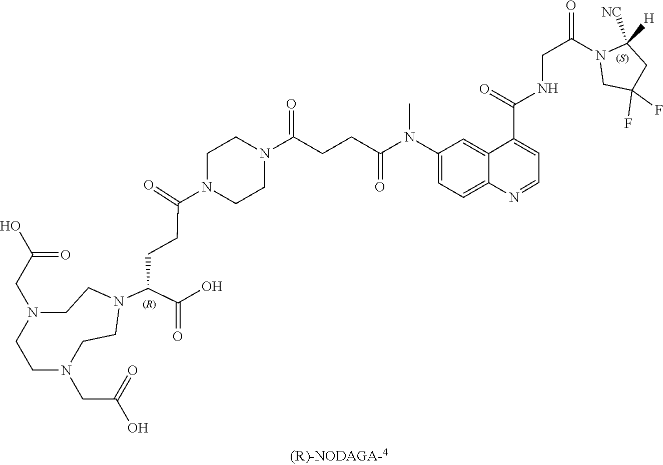

Step 1: To a solution of compound 4 (40.0 mg, 73.8 μmol) in DMF (0.50 mL) was added DIEA (9.55 mg, 73.8 μmol), HATU (57.6 mg, 110 μmol), and NODAGA-Tris(tBu) (48.1 mg, 88.6 μmol). The mixture was stirred at 25° C. for 1 hr. LCMS showed one peak of desired MS was detected. The mixture was concentrated in vacuum. The residue was purified by prep-HPLC (column: Waters Xbridge Prep OBD C18 150*40 mm*10 um; mobile phase: [water (NH₄HCO₃)-ACN]; B %: 50%-90%, 8 min) to obtain compound S (28.0 mg, 35.5% yield) as a white solid.

Step 2: Compound S (28.0 mg, 26.2 μmol) was taken up into a microwave tube in HFIP (4.41 mg, 26.2 μmol). The sealed tube was heated at 100° C. for 48 hrs under microwave. LCMS showed compound S consumed and one peak of desired MS was detected. The mixture was concentrated in vacuum. The residue was purified by prep-HPLC (column: Phenomenex Luna C_{18 75*30} mm*3 um; mobile phase: [water (TFA)-ACN]; B %: 5%-30%, 8 min) to obtain (R)-NODAGA-4 (9.01 mg, 36.9% yield, 96.6% purity, TFA) as an off-white solid. ¹H NMR: (400 MHz, D₂O) δ 9.10 (d, J=4.8 Hz, 1H), 8.31-8.27 (m, 2H), 8.00-7.97 (m, 2H), 5.15-5.12 (m, 1H), 4.35 (s, 2H), 4.26-4.22 (m, 1H), 4.17-4.15 (m, 1H), 3.75 (s, 4H), 3.60-3.50 (m, 9H), 3.22-3.09 (m, 18H), 2.67-2.58 (m, 6H), 2.07-1.96 (m, 2H). LCMS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, Product RT=1.640 min)

### 2.5: Synthesis of 2,2′-((R)-7-(1-carboxy-4-(4-(3-((4-((2-((S)-2-cyano-4,4-difluoropyrrolidin-1-yl)-2-oxoethyl)carbamoyl)quinolin-6-yl)(methyl)amino)propyl)piperazin-1-yl)-4-oxobutyl)-1,4,7-triazonane-1,4-diyl)diacetic acid (NODAGA-FAPI-46)

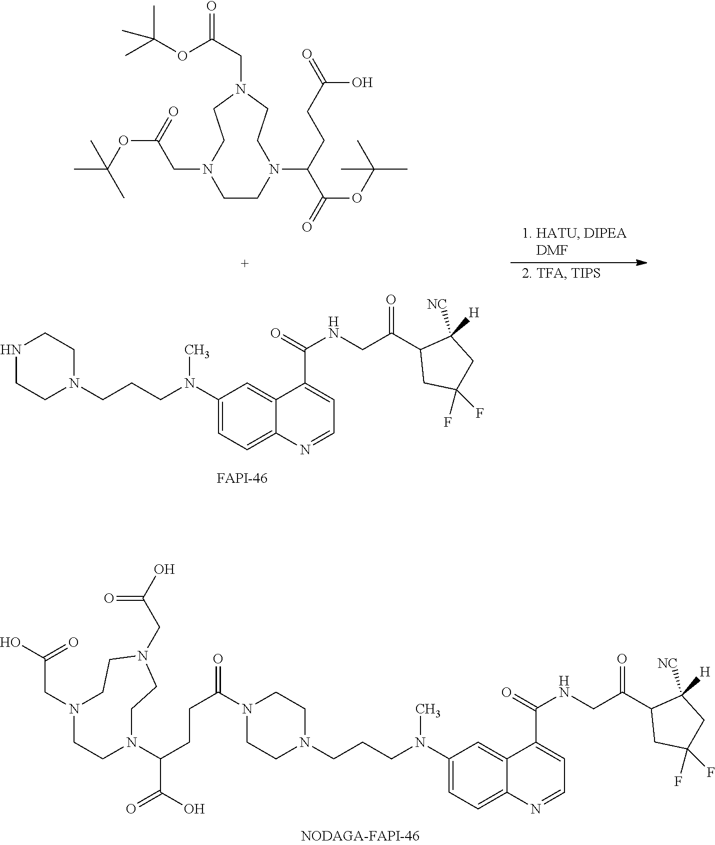 [SMILES: CC(C)(C)OC(=O)CN1CCN(CC(=O)OC(C)(C)C)CCN(C(CCC(=O)O)C(=O)OC(C)(C)C)CC1 ; CN(CCCN1CCNCC1)c1ccc2nccc(C(=O)NCC(=O)C3CC(F)(F)C[C@H]3C#N)c2c1 ; CN(CCCN1CCN(C(=O)CCC(C(=O)O)N2CCN(CC(=O)O)CCN(CC(=O)O)CC2)CC1)c1ccc2nccc(C(=O)NCC(=O)C3CC(F)(F)C[C@H]3C#N)c2c1]

(S)—N-(2-(2-cyano-4,4-difluoropyrrolidin-1-yl)-2-oxoethyl)-6-(4-oxo-4-(piperazin-1-yl)butanamido)quinoline-4-carboxamide, (R)-NODAGA(tris)tBu and HATU were dissolved in DCM+100 μL of DMF. DIPEA was added dropwise and the reaction was stirred for 2 h until completion (checked via LC/MS, method 15 to 80% in ACN). When no starting material was left and only a peak related to the product mass was observable (m/z=1025), TIPS and TFA (600 μL) were added. After 48 h, the reaction was complete. The crude was purified via HPLC (10-65% CAN in 15 min, rt=9.5) to afford 6.8 mg of a red powder (Yield: 36%).

### Example 3: Cold Labelling

^{nat}Cu-NODAGA-1 and ^{nat}Cu-NODAGA-3

The ^{nat}Cu complexes were prepared by incubating each conjugate with 1.5-fold excess of ^{nat}CuCl₂×2 H₂O in ammonium acetate buffer, 0.5 M, pH 8 at 95° C. for 15 min. Uncomplexed ^{nat}Cu ions were eliminated by SepPak C-18 purification. The ^{nat}Cu-complexes were eluted with methanol, evaporated to dryness, re-dissolved in water and lyophilized. The purity of all complexes was confirmed by liquid chromatography and mass spectrometry (LC-MS). Table 1 presents the retention time (t_{R}), and the obtained mass (mass-to-charge ratio, m/z) of the ion [M+2H]²⁺ in comparison to the theoretical mass, confirming the identity of the formed ^{nat}Cu-complexed conjugates. The analysis was performed on a LC-MS (Shimadzu LC2020) system using Gemini C6 Phenyl 5 μm, 250×4.6 mm column and a gradient of 15-80% acetonitrile (0.1% TFA)/water (0.1% TFA) in 15 min, at a flow rate of 2 mL/min. LC-MS chromatogram data are provided in Table 1A.

TABLE 1A
Analytical for ^{nat}Cu conjugates

| ^{nat}Cu-complexed conjugates | m/z calculated | m/z measured | t_{R} (min) |
|---|---|---|---|
| NODAGA-1 | 858.9 | 859 | 6.28 |
| ^{nat}Cu-NODAGA-1 | 920.2 | 920.3 | 9.77 |
| NODAGA-3 | 884.9 | 885 | 9.97 |
| ^{nat}Cu-NODAGA-3 | 946.9 | 946.5 | 9.94 |

m/z = mass-to-charge ratio of the ion [M + H]⁺;
t_{R} = retention time

^{nat}Cu-NODAGA-2 and ^{nat}Cu-NODAGA-4

The ^{nat}Cu complexes were prepared by incubating 1-1.5 mg of each conjugate with a 1.5-fold excess of CuCl₂ in 125-300 μL of ammonium acetate (0.5 M, pH 8). A pH check was performed in order to guarantee the necessary conditions for the reaction (pH≥5). The reaction mixture was incubated for 10 min at room temperature. Free metal ions were eliminated via HPLC (Shimadzu SCL-40, Phenomenex Jupiter Proteo C12 (90 Å, 250×4.6 mm) column using the gradient 15-80% B in 8 min (A=H₂O [0.1% TFA], B=ACN [0.1% TFA]) with a flow rate of 5 ml/min).

TABLE 1B
LC/MS, HPLC profile of the described compounds.

| Compound | m/z calculated | m/z measured | tR (min) |
|---|---|---|---|
| NODAGA-2 | 872.88 | 873 | 10.62 |
| ^{nat}Cu-NODAGA-2 | 934.41 | 934 | 9.44 |
| NODAGA-4 | 898.92 | 899 | 10.73 |
| ^{nat}Cu-NODAGA-4 | 960.45 | 960 | 10.46 |

m/z = mass-to-charge ratio of the ion [M + H]⁺;
t_{R} = retention time

### Example 4: High Purity Copper-61 (⁶¹Cu)

1. Methods of Making a Coin

Preparation of Buffer Solution

Ammonium Chloride (4.6 g, Aldrich: 326372, Trace Select) was weighed into a clean (no metal) Falcon Tube (50 mL), and the previously cleaned magnetic stirring bar was added. 6 mL of Trace Select water (Honeywell 95305) was added in one aliquot to flush walls of the Falcon in case any salt stuck to the Falcon tube walls. 1 mL of ammonium hydroxide 28% (Sigma 338818) was added with a 1000 μL pipette with a respective pipette tip, 8× times. The lid of the Falcon was closed, and the Falcon was, in turns, vortexed (1-2 minutes) (immersion in an ultra-sonic bath was a possible alternative for 1-2 minutes) and shaken, until all salt was dissolved. The Falcon tube can also be warmed (e.g., by rolling between hands) to improve solubility, temperature (e.g., around 23° C., preferably between 23-25° C.). After complete dissolution of the salt, the pH acceptance criteria, pH range 9.28-9.62, was verified by pH measurement of the solution at RT, e.g., with and electronic pH meter. The Falcon tube was closed with parafilm and stored at room temperature. Prior to use, any solid salt formation was redissolved.

Preparation of Nickel Nitrate Plating Solution

A 50 mL glass beaker was washed with nitric acid (Trace Select) followed by water (Trace Select). In a fume hood, the beaker was dried by placing it on a heating plate set to 150° C. To the beaker was added 210 mg of natural (isotopic distribution) nickel (powder, Sigma-Aldrich≤50 μm, 99.7% trace metals basis, essentially free from any impurities, except iron. The copper impurity amounts to <0.3 ppm.) were weighed into the beaker and 4 mL of 65% nitric acid were added using a pipette. The beaker was placed back on the active heating plate and the stirring was set to 300 rpm. Proper ventilation of the fume hood was confirmed (evolution of NO₂). During the dissolution, the solution turned green. The solution was reduced by evaporation to a volume of 600 μL and taken from the heating plate to cool down to room temperature. The remaining solution was transferred to a 50 mL metal-free Falcon tube. The glass beaker was rinsed with a total of 2.8 mL of Trace Select water, in steps of 0.8 mL, 1 mL, and 1 mL, where each step was transferred to the Falcon tube before the adding the next washing fraction. Buffer solution (4 mL), 11 mL of Trace Select water, and 3 mL of ammonium hydroxide 28% (Sigma 338818) were added to the Falcon tube. The pH of the solution was measured and adjusted to the required pH by adding ammonium hydroxide 28% (Aldrich 338818) using sterile B-Braun syringes.

Examples of Suitable Starting Material to Prepare ⁶⁰Ni And ⁶¹Ni Electroplating Solutions

The following Tables 2-4 are example lots of ⁶⁰Ni and ⁶¹Ni (certificate as provided by Isoflex, USA, March 2018):

TABLE 2

| | | | | | | | | | | | |
|---|---|---|---|---|---|---|---|---|---|---|---|
| Isotope | ⁶¹Ni | | | | | | | | | | |
| Enrichment | 86.20% | | | | | | | | | | |
| Form | Metal | | | | | | | | | | |
| | ingot/powder | | | | | | | | | | |
| Certificate | 6275 | | | | | | | | | | |
| Isotopic distribution | Isotope | Ni-58 | Ni-60 | Ni-61 | Ni-62 | Ni-64 | | | | | |
| | Content (%) | 1.17 | 0.8 | 86.2 | 11.7 | 0.14 | | | | | |
| Chemical admixtures | Element | Al | Bi | Ca | Cd | Co | Cr | Cu | Fe | K | Mg |
| | Content | 10 | <10 | 20 | 10 | <10 | <10 | 20 | 40 | <10 | <50 |
| | (ppm) | | | | | | | | | | |
| | Element | Mo | Mn | Na | Pb | Si | Sn | Zn | | | |
| | Content | <8 | <50 | <10 | <10 | 20 | 30 | 50 | | | |
| | (ppm) | | | | | | | | | | |

TABLE 3

| | | | | | | | | | | | |
|---|---|---|---|---|---|---|---|---|---|---|---|
| Isotope | ⁶¹Ni | | | | | | | | | | |
| Enrichment | 99.39% | | | | | | | | | | |
| Form | Metal | | | | | | | | | | |
| | powder | | | | | | | | | | |
| Certificate | TBD/not | | | | | | | | | | |
| | specified | | | | | | | | | | |
| Isotopic | Isotope | Ni-58 | Ni-60 | Ni-61 | Ni-62 | Ni-64 | | | | | |
| distribution | Content (%) | 0.01 | 0.29 | 99.39 | 0.29 | 0.02 | | | | | |
| Chemical | Element | Al | Co | Cr | Cu | Fe | Mg | Mn | Pb | Si | Ti |
| admixtures | Content | 12 | <10 | <10 | 14 | <10 | <10 | <10 | <10 | <10 | <10 |
| | (ppm) | | | | | | | | | | |
| | Element | Zn | C | S | | | | | | | |
| | Content | <10 | 157 | <10 | | | | | | | |
| | (ppm) | | | | | | | | | | |

TABLE 4

| | | | | | | | | | | | |
|---|---|---|---|---|---|---|---|---|---|---|---|
| Isotope | ⁶¹Ni | | | | | | | | | | |
| Enrichment | 99.31% | | | | | | | | | | |
| Form | metal | | | | | | | | | | |
| | powder | | | | | | | | | | |
| Certificate | TBD/not | | | | | | | | | | |
| | specified | | | | | | | | | | |
| Isotopic | Isotope | Ni-58 | Ni-60 | Ni-61 | Ni-62 | Ni-64 | | | | | |
| distribution | Content (%) | 0.21 | 99.31 | 0.46 | 0.015 | 0.005 | | | | | |
| Chemical | Element | Al | Co | Cr | Cu | Fe | Mg | Mn | Pb | Si | Ti |
| admixtures | Content | <10 | 70 | 20 | 25 | <10 | <10 | <10 | <10 | 15 | <10 |
| | (ppm) | | | | | | | | | | |
| | Element | Zn | C | S | P | | | | | | |
| | Content | 15 | 114 | 20 | 30 | | | | | | |
| | (ppm) | | | | | | | | | | |

The samples of natural nickel from Sigma-Aldrich were essentially free from any impurities, except iron. The copper impurity amounts to <0.3 ppm. Certificates of analysis are described below. Additional suitable sources of natural Ni include:
Nickel powder, <50 μm, 99.7% trace metals basis
Nickel rod, diam. 6.35 mm, =99.99% trace metals basis
Nickel foil, thickness 0.5 mm, 99.98% trace metals

Preparation of Zinc Nitrate Plating Solution

A 50 mL glass beaker was washed with nitric acid (Trace Select) followed by water (Trace Select). In a fume hood, the beaker was dried by placing it on a heating plate set to 150° C. 210 mg of natural (isotopic distribution) zinc (zinc powder, Sigma-Aldrich≤10 μm, >98%) was weighed into the beaker and 4 mL of 65% nitric acid was added using a pipette. The beaker was placed back on the active heating plate and the stirring was set to 300 rpm. Proper ventilation of the fume hood was confirmed (evolution of NO₂). During the dissolution, the solution turned green. The solution was reduced by evaporation to a volume of ≈600 μL and taken from the heating plate to cool down to room temperature. The remaining solution was transferred to a 50 mL metal-free Falcon tube. The glass beaker was rinsed with a total of 2.8 mL of Trace Select water, in steps of 0.8 mL, 1 mL, and 1 mL, where each step was transferred to the Falcon tube before the adding the next washing fraction. 4 mL of the buffer solution (prepared above), 11 mL of Trace Select water, and 3 mL of ammonium hydroxide 28% (Sigma 338818) were added to the Falcon tube. The pH of the solution was measured and adjusted to the required pH by adding ammonium hydroxide 28% (Aldrich 338818) using sterile B-Braun syringes.

Electroplating the Backing Surface

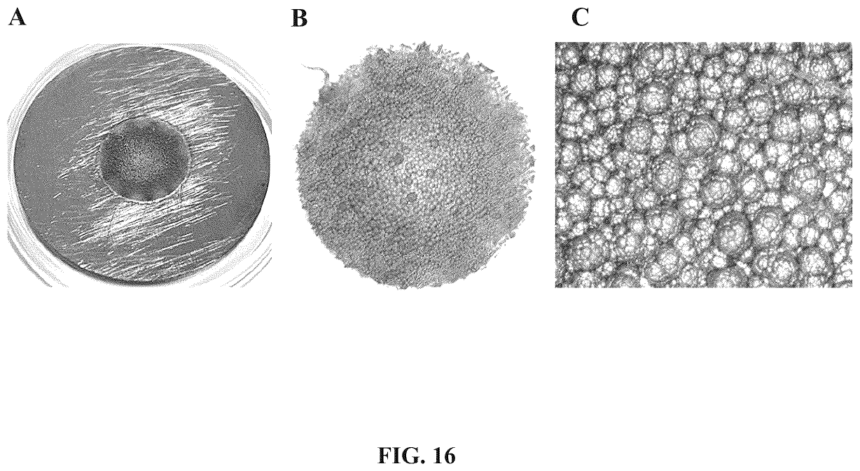

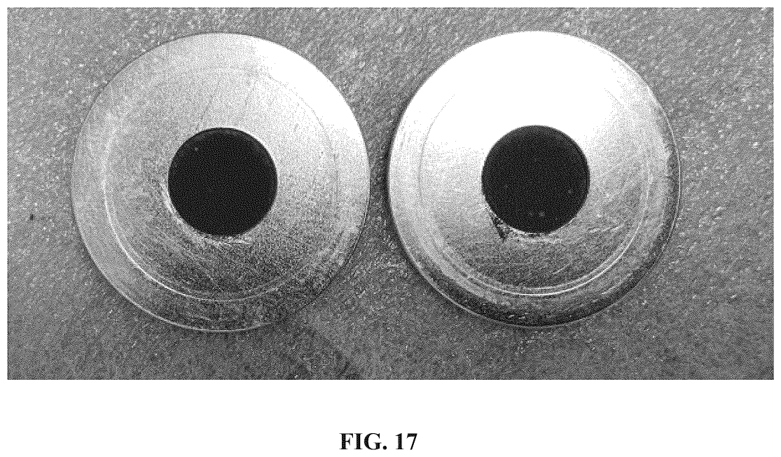

A disc shaped niobium backing was obtained from high purity Nb as described herein and (28 mm×1.0 mm) was cleaned with ethanol (high-purity) and inserted in a Comecer Electroplating Unit V21204. A platinum wire anode was positioned so that the distance relative to the coin surface was between about 1 and 3 mm, adjusted by a polymer spacer. The coin mass was determined to be 5.25 grams. Niobium backing (22 mm×1.0 mm weighs 3.3 g). The plating solution was charged to the electrolyte container and attached to the apparatus. The voltage was set to 4.5V. The current reading after 5 min stabilization was 180 μA. The duty cycle for pump was set to 45%. The plating liquid turned from blue to transparent, slow decrease of current to 160 μA was observed over the period of 120 minutes. The plating process was stopped. The coin was taken out of the electrolytic cell and its weight was measured. The coin also underwent microscopic evaluation using a DINOLite digital microscope to observe the crystal structure and homogeneity of the surface (FIG. 16). The coin (FIG. 17) was stored in a metal-free Falcon tube under a nitrogen atmosphere.

Upon completion of electroplating, the coin underwent a microscopic evaluation using a DINOLite digital microscope to observe the crystal structure and homogeneity of the surface. As can be seen in FIG. 16 (panels A-C), a homogenous target coating having durable adhesion was obtained.

2. General Guidelines for High-Purity [⁶¹Cu]Cl₂ Production

The purpose of this example was to enable the bulk production of Copper-61 (⁶¹Cu) from the deuteron irradiation of natural nickel and/or enriched ⁶⁰Ni. This effort was a proof of concept, and, therefore, there were no benchmarked specifications for ⁶¹Cu. However, we optimize target performance, target geometry/material use, irradiation parameters, and chemical processing methods to produce [⁶¹Cu]CuCl₂ following enriched ⁶⁰Ni irradiation, or, scaled accordingly for ^{nat}Ni irradiation. There were no pharmacopoeia specifications for radio-copper explicitly, however, test QC methods include assessment of radionuclidic purity and apparent molar activity (to demonstrate usability of the extracted [⁶¹Cu]CuCl₂).

This example considers use of two different types of targets, natural nickel (^{nat}Ni) targets and highly enriched Nickel-60 (⁶⁰Ni) targets both of which were suitable for deuteron bombardment. However, ^{nat}Ni was cost-effective and available in high-purity while ⁶⁰Ni was costly and requires efficiency measures. If even higher yields were desired, target preparation efforts may be directly translated into the proton-based ⁶¹Ni(p,n)⁶¹Cu route, however, given the cost of enriched ⁶¹Ni (c.a. $25 USD/mg), such an approach imposes the need for target recycling.

The set of guidelines below enables all types of targets in the production of ⁶¹Cu, including the production of high-purity [⁶¹Cu]CuCl₂ from the Nb coins with a Zn or Ni (any isotopic enrichment) coating electroplated thereon as provided herein. Specific details are also provided for deuteron and proton irradiations, respectively. This protocol was followed to generate the [⁶¹Cu]Cl₂ compositions evaluated herein.

| Target Backing | Flat coin - disc-shaped. The dimensions of the target backing form are: | |
|---|---|---|
| Geometry | Coin backing: | |
| | Diameter Ø = 20 − 30 ± 0.1 mm | |
| | Thickness H = 1.5 mm | |
| | Target Ni layer or coating | |
| | a. Diameter 13 mm (deuteron) or 10 mm (proton) | |
| | b. Mass 70-100 mg, e.g, around 100 ± 40 mg (deuteron) or | |
| | around 50 ± 20 mg (proton) | |
| | c. Thickness (H) full density (d = 8.9) | |
| | i. Hₘᵢₙ = 0.1 mm; Hₘₐₓ = 0.14 mm corresponding to | |
| | 70-100 mg deposited | |
| | Tolerances/finishes unless otherwise stated are as follows: | |
| | Surface finish: Ra 1.6 | |
| | General tolerance: ISO 2768-m | |
| | Sharp edges and corners according to ISO 13 715 | |
| Target Backing | Optional - Surface treated with abrasion by pink corundum grindstone - | |
| Surface | free of impurities | |
| Target Backing | Niobium foil, 99.8% (metals basis), 1.0 mm (0.04 in) thick, annealed, | |
| Material | Stock No.: 10257 | |
| | Lot No.: C15P07 | |
| | Element | ppm |
| | Carbon | 24 |
| | Hydrogen | 1 |
| | Molybdenum | 2 |
| | Nickel | 4 |
| | Silicon | 1 |
| | Titanium | 2 |
| | Zirconium | 3 |
| | Iron | 1 |
| | Hafnium | 2 |
| | Nitrogen | 14 |
| | Oxygen | 56 |
| | Tantalum | 785 |
| | Tungsten | 4 |
| Target Backing | Niobium 99.9% typical certificate of analysis results, Goodfellows | |
| Material | Product nr. 931-627-20 | |
| | Element | ppm |
| | B | <10 ppm |
| | Ni | <5 ppm |
| | O | 100 ppm |
| | Si | 100 ppm |
| | Zr | <10 ppm |
| | Ta | 500 ppm |
| | H | <10 ppm |
| | W | <100 ppm |
| | C | 25 ppm |
| | N | 20 ppm |
| | Fe | 30 ppm |
| | Cu | <5 ppm |
| | Mo | 10 ppm |
| | Ti | <10 ppm |

| | |
|---|---|
| Transfer system | As the target can be automatically transferred to/from the cyclotron by |
| compatibility | means of a pneumatic target transfer system, it was critical that the |
| | deposited Ni was robust to direct air flow and abrupt mechanical |
| | movements. |
| | In certain embodiments, the target coating remains adhered to the backing |
| | during pneumatic transfer both to and from the cyclotron. Such a |
| | pneumatic system was typically fed by a compressed air connection of ~6-7 |
| | bar, and at minimum, 360 SLPM flow. Such a system was “push-push”, and |
| | therefore, compressed air was typically blown on both the front and rear |
| | sides of the coin, respectively, depending on the direction of transfer. The |
| | coin came to an abrupt stop as it reached the target station or hotcell. |
| | In certain embodiments, suitable tests that indicate target durability include |
| | the following, whereby the total mass loss for all tests combined should be |
| | negligible (e.g. <1 mg): Visual inspection, gentle knocking/tapping on a |
| | countertop on top of white paper to check for loosening of target coating |
| | grains, gently rubbing an acid-washed Teflon spatula against the deposited |
| | target coating and checking for loosening of target coating grains, and/or |
| | placing and gently pressing down on a piece of Scotch tape against the |
| | target coating. |
| | If there was access to the cyclotron apparatus, it was recommended to |
| | transfer the coin back/forth multiple times and ensure target coating |
| | stability (i.e., no mass loss). Such a test may be performed with a degrader |
| | in place. |
| Method of | Electrodeposition from bath with a significantly high pH (e.g., 9.9-10.8) |
| Production | |
| Target Metal | To withstand the deposited beam power, the target metal was preferably |
| Form | metallic nickel (not, e.g., nickel oxide). |
| | Depending on the means of target preparation (e.g., electroplating), the raw |
| | nickel starting material need not necessarily be metallic. However, methods |
| | used for preparing ^{nat}Ni targets should ultimately be directly translatable to |
| | preparation of ⁶⁰Ni or ⁶¹Ni targets. At present, it was understood that |
| | enriched Ni was typically in the form of a salt. |
| Target Additives | The use of binders must not necessary be avoided if they are absent of the |
| | final metallic coin and if an assessment on a case-by-case basis to |
| | understand potential impact to product quality has been done (e.g. ICP-MS |
| | on the binder material). |
| | Any reagents used for target preparation (e.g., electroplating reagents) must |
| | be of the highest quality, in particular, with regards to trace metals. |
| Metal Content | Preferably, the highest grades of reagents should be used, to avoid trace |
| | metals contamination of the target coating, as more than a tenth of a |
| | microgram per 100 mg of target metal (that is, 1 ppm of the target metal) is |
| | already a significant contamination that may render the coin unusable for |
| | production of high-purity radionuclides. In the case of the production of |
| | radiocopper it is not accepted to add more than 0.1 ppm of cold Cu as this |
| | would reduce the purity of the prepared radionuclide composition. |
| | Preferably, max level of impurities allowed to be added by the process to |
| | the initial nickel: |
| | Copper (Cu): 0.1 ppm |
| | High affinity metals (Ga, Lu, Pb, Y): 0.1 ppm |
| | Zinc and cobalt (Zn, Co): 0.3 ppm |
| | Transition and other metals (Cd, Cr, Al, Mn, Mo, Sn, Ti, V . . .): |
| | 1 ppm on a case by case |
| | Iron (Fe): 10 ppm |
| | Family I and II (K, Ba, Mg, Be . . .): 1000 ppm |
| | The metal coins were analyzed on a batch per batch basis by dissolution in |
| | nitric acid to assess the metal contamination within the coin that were not |
| | found in the starting nickel metal and thus originate from the process. |
| | The amount suggested above were a good, albeit not strict, guide since |
| | chemical purification following irradiation will, in turn, further remove |
| | some of these impurities. The ultimate specification on this front will |
| | therefore be an iterative process as the Cu/Ni separation chemistry is |
| | refined. However, the process shall not significantly add impurities that |
| | were not in the originating pure nickel material. |
| | Cold Cu should be minimized in the deposited Ni since this will follow the |
| | chemistry of any ⁶¹Cu and cannot be separated post-irradiation. Any such |
| | cold Cu will directly compete with ⁶¹Cu during radiolabeling. Methods of |
| | removing Cu from the dissolved target metal are well known. |
| Density of Target | To withstand the deposited beam power, the Ni target should be of |
| | reasonably high volumetric density (e.g., approximately ≥90% or, ≥8.0 |
| | g/cm³). |
| Power Rating | The power rating for the target, including the combined deposited Ni and |
| | plate should be: |
| | ≥420 W (deuterons) |
| | ≥820 W (protons) |
| Loading Mass of | The loading mass vs. the deposited mass of Ni (i.e., deposition efficiency) |
| Target | relates not to technical specifications, but rather, to cost. In the case of ^{nat}Ni |
| | deposition, loading efficiency did not have a significant impact on the cost |
| | of ⁶¹Cu. However, losses should be minimized in considering the translation |
| | to enriched ^{6x}Ni. For ⁶⁰Ni, losses should be maintained below ~10%, and |
| | for ⁶¹Ni, below ~1%. Some techniques such as magnetron sputtering are |
| | thus not possible for enriched nickel but are satisfactory for ^{nat}Ni. |
| Mass/thickness | For deuterons (i.e., ^{nat}Ni or ⁶⁰Ni), the thickness should be appropriate for |
| of Nickel | stopping the deuterons, with a maximum 10% variability in material |
| | deposition. Such thicknesses equate to: |
| | ≥100 μm (assuming 100% density) |
| | ≥70 mg or ≥89 mg/cm² (assuming 10 mm diameter) |
| | For protons (i.e., ⁶¹Ni), one may wish to selectively limit the deposited |
| | material to optimize the balance between material cost, yield, and backing |
| | material activation. With a maximum 10% variability in material |
| | deposition, four examples are noted below. |
| | ⁶¹Ni Scenario #1 (11→9 MeV) |
| | 78 μm (assuming 100% density) |
| | 55 mg or 69 mg/cm² (assuming 10 mm diameter well) |
| | ⁶¹Ni Scenario #1 (12→8 MeV) |
| | 155 μm (assuming 100% density) |
| | 108 mg or 138 mg/cm² (assuming 10 mm diameter well) |
| | ⁶¹Ni Scenario #1 (13→7 MeV) |
| | 233 μm (assuming 100% density) |
| | 163 mg or 208 mg/cm² (assuming 10 mm diameter well) |
| | ⁶¹Ni Scenario #1 (13→4 MeV) |
| | 309 μm (assuming 100% density) |
| | 216 mg or 275 mg/cm² (assuming 10 mm diameter well) |
| Isotopic | The ^{6x}Cu radioisotopes which will be coproduced during production of ⁶¹Cu |
| enrichment | (t ½ = 3.339 h) include: |
| | ⁵⁷Cu (t ½ = 0.196 s) ⁵⁸Cu (t ½ = 3.204 s) |
| | ⁵⁹Cu (t ½ = 81.5 s) ⁶⁰Cu (t ½ = 23.7 m) |
| | ⁶²Cu (t ½ = 9.673 m) ⁶⁴Cu (t ½ = 12.701 h) |
| | From a practical handling point of view, all but ⁶⁰Cu and ⁶⁴Cu are likely to |
| | decay prior to use. Only ⁶⁴Cu will have any impact on the possible shelf-life |
| | of ⁶¹Cu. |
| | In addition to the production of Cu radioisotopes, other radionuclides (e.g., |
| | Co and Ni) may also be produced, the ratio of which will depend on the |
| | isotopic composition, and whether undergoing deuteron or proton irradiation. |
| | As these byproducts are chemically different from copper, such radionuclides |
| | may be removed during ⁶¹Cu purification/processing. For example, The ⁶¹Cu |
| | was purified from metal and radiometal impurities via a GE Healthcare |
| | FASTlab 2 module through a tributyl phosphate resin cartridge and a tertiary- |
| | amine-based weak ionic exchange resin containing long-chained alcohols. |
| Any other | Niobium is preferred over silver for its better resistance to corrosion, its low |
| requirements | amount of activation on irradiation and for its high melting temperature that |
| | permits the deposit of nickel by other processes such as melting or heat |
| | sintering. However, silver possesses a higher thermal conductivity and may |
| | be suitable for certain embodiments. |
| | For target backing manufacture, the following sheet of niobium is suitable |
| | for laser cutting: |
| | http://www.Goodfellow.com |
| | NB000400 Niobium Foil, Size: 150 × 150 mm Thickness: 1.5 mm, |
| | Purity: 99.9%, Temper: Annealed, Quality: LT |
| | From one sheet up to 25 target backings can be manufactured. |

Purification and Characterization of [⁶¹Cu]CuCl₂ and Waste Streams

The solid target irradiated material was dissolved in a total volume of 7 mL of 6 M HCl with the addition of 30% hydrogen peroxide via a dissolution chamber. Separation and purification were accomplished using a cassette-based FASTlab platform using a TBP (tributylphosphate-based) resin (1 mL) (particle size 50-100 μm; pre-packed, Triskem) then a weakly basic (tertiary amine; TK201) resin (2 mL) (particle size 50-100 μm; pre-packed, Triskem), each of which were pre-conditioned with H₂O (7 mL) and HCl (10M, 7 mL). The cassette reagent vials were prepared using concentrated HCl (Optima Grade, Fischer Scientific), NaCl (ACS, Fischer Scientific) and milli-Q water (Millipore system, 18 M2-cm resistivity). 6M HCl (2×4.2 mL), 5M NaCl in 0.05 M HCl (4.2 mL). The subsequent ⁶¹Cu was then purified with two subsequent ion exchange resins in a FASTlab synthesis unit.
1) The acid-adjusted dissolution solution (approx. 7 mL) was loaded over both columns in series and directed into a “Ni collection fraction”. The TBP resin acted as a guard column as it quantitatively retained Fe³⁺ ions, while the Cu²⁺ and Co²⁺ complexes were quantitatively retained on the tertiary amine (TK201) resin.
2) Both columns were washed with 6M HCl (4 mL) to maximize Ni recovery for future recycling.
3) The TK201 column was washed with 4.5M HCl (5.5 mL) to elute the majority of cobalt salts.
4) The TK201 column was washed with 5M NaCl in 0.05M HCl (4 mL) to decrease residual acid on the resin and further remove any residual cobalt salts.
5) The TK201 column was washed with of 0.05M HCl (3 mL) to quantitatively elute the [⁶¹Cu]CuCl₂.

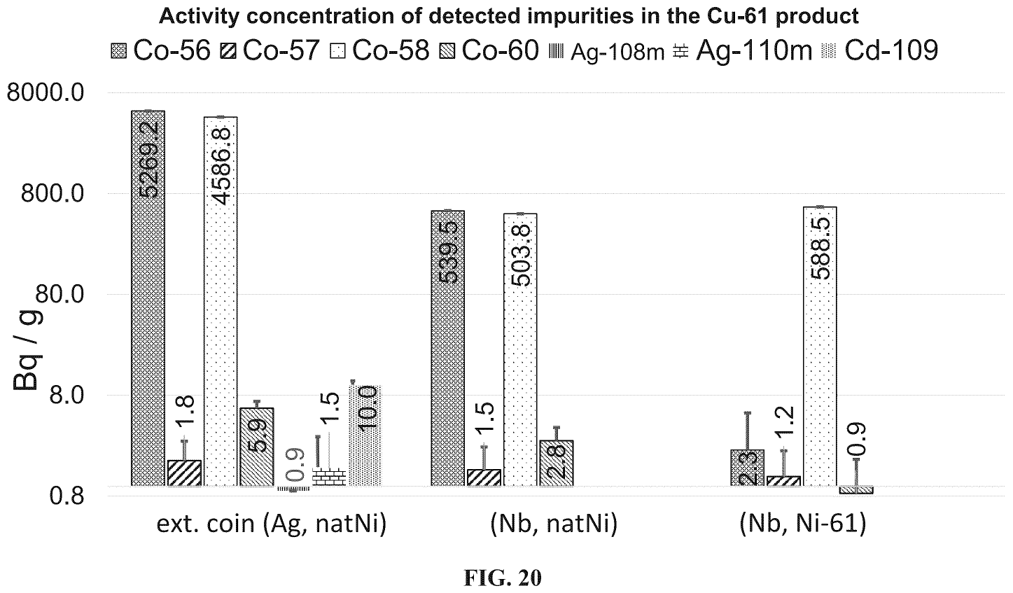

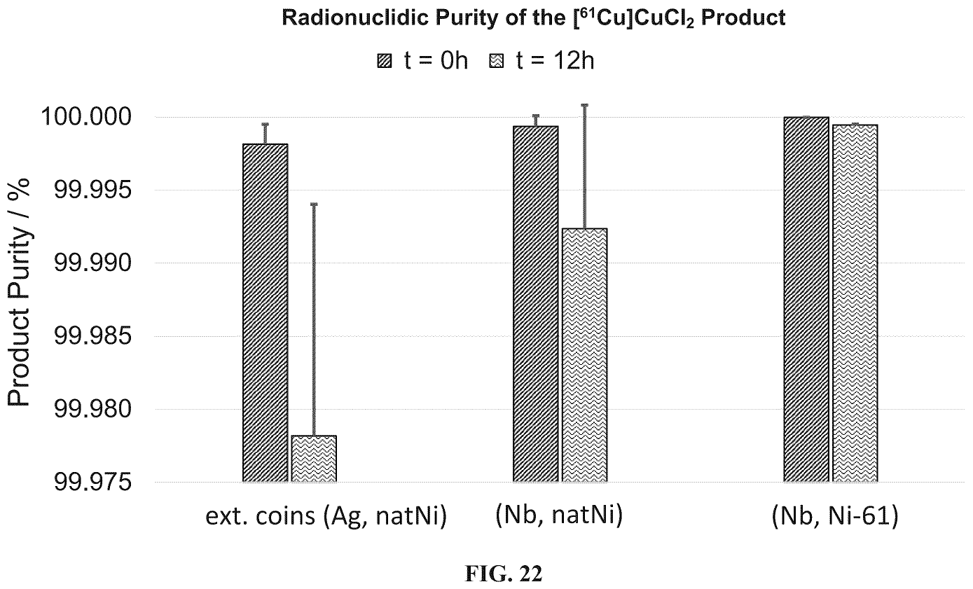

The resulting [⁶¹Cu]CuCl₂ solution of the plated material had an average activity of 1.0-4.5 GBq (FIG. 20). This activity was measured using a dose calibrator from Comecer and its radionuclidic purity by a gamma spectrometer at PSI in Switzerland (FIG. 22).

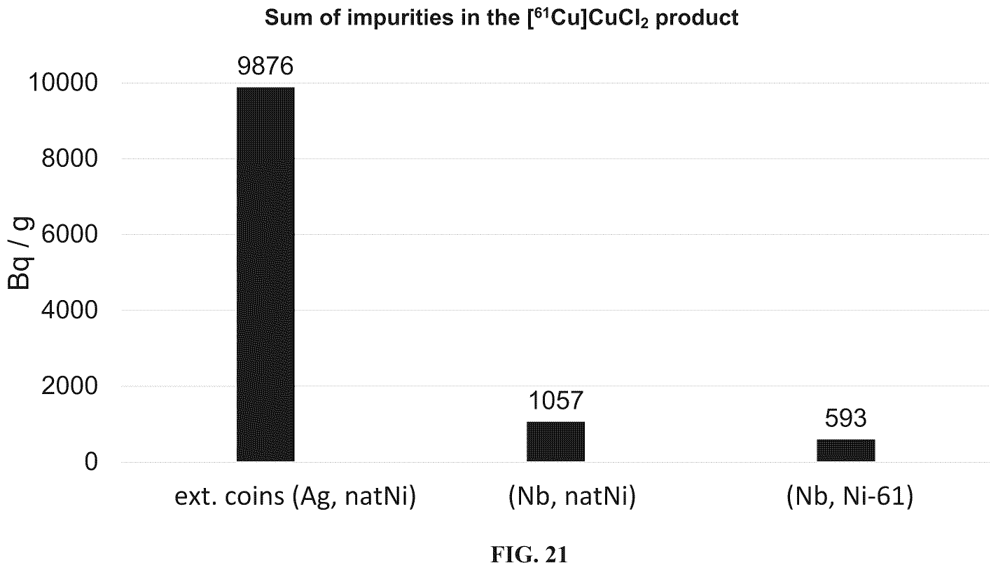

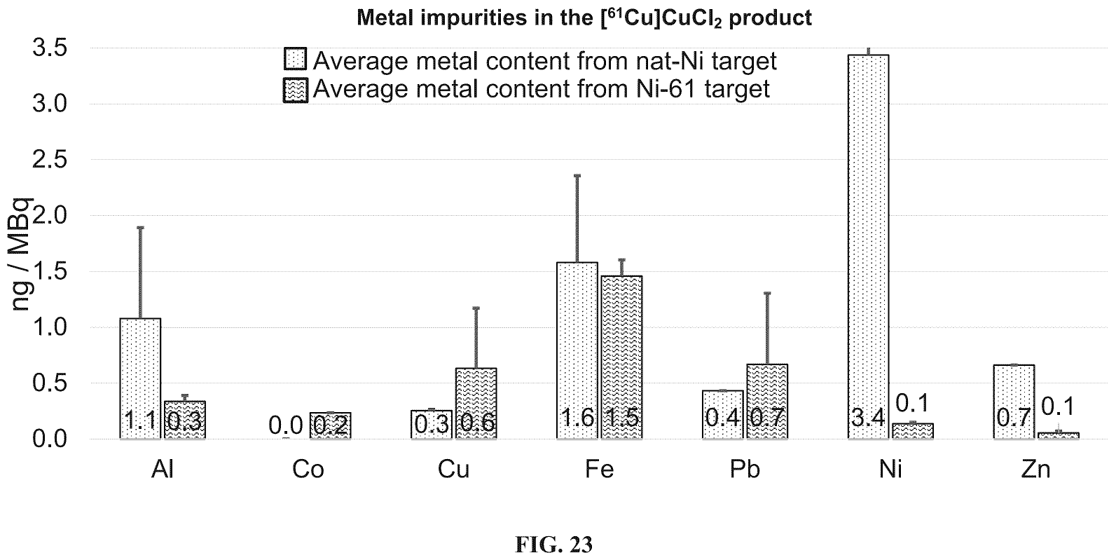

Gamma spectrometry measurements were performed to identify any radionuclidic impurities, particularly long-lived radionuclides. These results indicate an 89.3% and 94% reduction in impurities for ^{nat}Ni and ⁶¹Ni on niobium backing materials with respect to silver backing materials when utilizing the methods disclosed herein (FIG. 20 and FIG. 21). ICP-MS measurements were performed on the product of cold dissolutions by Labor Veritas in Switzerland to monitor elemental impurities present in product according to ICH-Q3D (FIG. 23). All detected impurities were within regulated ICH-Q3D concentrations (see ICH-Q3D Guidelines, pg 25).

The plating of highly enriched ⁶¹Ni was also enabled with the same plating parameters as described above, for a higher yield and industrial production using proton irradiation (typically at 80 μA to 100 μA, 13 MeV protons for 1 hour to 2 hours and up to one half-life of ⁶¹Cu).

Purity and Activity Evaluations of [⁶¹Cu]CuCl₂ Compositions Prepared from ^{nat}aNi(d,n)⁶¹Cu and ⁶⁰Ni(d,n)⁶¹Cu Using Nb-Backed Coins.

This example presents information on the activity of the produced ⁶¹Cu generated using the Nb backing, Ni electrodeposited coins alongside cobalt radioisotopes that were produced with deuteron irradiation using the coin comprising a natural nickel target and the coin comprising enriched ⁶⁰Ni as target, i.e., ⁶¹Ni(d,n)⁶¹Cu and ⁶⁰Ni(d,n)⁶¹Cu, respectively. The irradiated materials were dissolved and purified as described above.

The obtained and purified [⁶¹Cu]Cu product and waste generated during purification from the products of deuteron irradiation of natural nickel/Nb coin and ⁶⁰Ni/Nb coin, respectively, was processed and analysed by gamma-spectrometry and presented below.

TENDL-2019 based thick target yield calculations using isotopic abundancy of natural nickel/Nb coin and enriched ⁶⁰Ni/Nb coin, respectively.

Radiocobalt Content

Table 5 contains activities of cobalt radioisotopes in the different fractions post FASTlab purification as a mean of three measurements (n=3 irradiations) using ^{nat}Ni/Nb target coin. The activities were extrapolated to a 3 h and 50 μA beam at EoB (end of bombardment)+2 h. The activity of [⁶¹Cu]CuCl₂ in these irradiations was determined experimentally and confirmed to be ˜80% of TENDL-2019 based estimates.

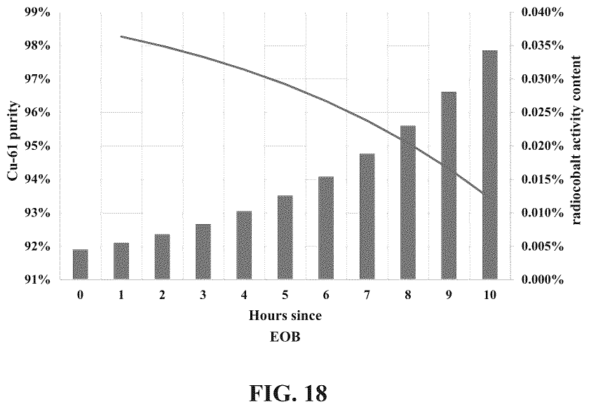

Activity of produced ⁶¹Cu for irradiation with deuteron at 8.4 MeV, 3 h at 50 μA at 80% efficiency (EoB+2 h): 3052 MBq. Also see FIG. 18 for the change in cobalt radioisotopes with time along with the corresponding change in ⁶¹Cu purity.

TABLE 5
Cobalt isotopes: natNi/Nb target coin

| | Cu fraction | Ni fraction | Co-waste I + II | Half-life |
|---|---|---|---|---|
| Radionuclide | [Bq] | [Bq] | [Bq] | [days] |
| ⁵⁶Co | 118345 | 2696 | 2458071 | 77 |
| ⁵⁷Co | 0 | 0 | 474 | 272 |
| ⁵⁸Co | 95395 | 2145 | 1940192 | 71 |
| ⁶⁰Co | 124 | 3 | 2602 | 1925 |

Table 6 contains calculated activities of cobalt radioisotopes that would be obtained by using 99% enriched ⁶⁰Ni as target metal. The activities were extrapolated to a 3 h and 50 μA beam at EoB (end of bombardment)+2 h. The activity of ⁶¹Cu was calculated accordingly.

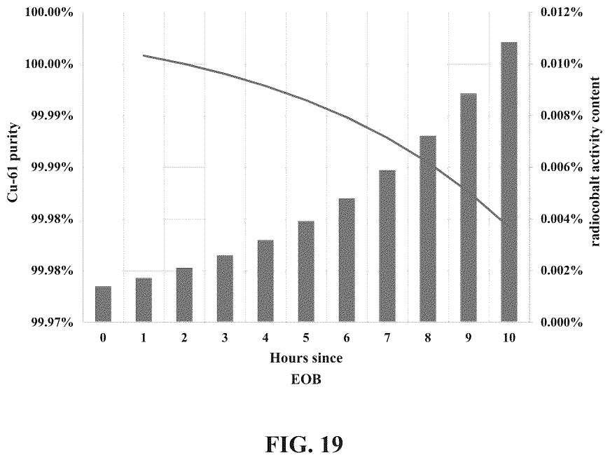

Activity of produced ⁶¹Cu with deuteron irradiation at 8.4 MeV, 3 h at 50 μA at 80% efficiency (EoB+2 h): 11,552 MBq. Also see FIG. 19 for the change in cobalt radioisotopes with time and the corresponding change in ⁶¹Cu purity.

TABLE 6
Cobalt isotopes: enriched ⁶⁰Ni/Nb target coin.

| | | | Separated | |
|---|---|---|---|---|
| | ⁶¹Cu fraction | Separated Ni | Co-waste I + II | Half-life |
| Radionuclide | [Bq] | [Bq] | [Bq] | [days] |
| ⁵⁶Co | 365 | 8 | 7583 | 77 |
| ⁵⁷Co | 0 | 0 | 1793 | 272 |
| ⁵⁸Co | 242909 | 5463 | 4940424 | 71 |
| ⁶⁰Co | 0.5 | 0 | 11 | 1925 |

Activity and Chemical Purity

Based on a combination of theoretical calculations and experimental results, the purity of [⁶¹Cu]CuCl₂ produced from deuteron irradiation of ^{nat}Ni/Nb target coin was compared with [⁶¹Cu]CuCl₂ from deuteron irradiation of enriched ⁶⁰Ni/Nb target coin.

In Table 7, the extrapolated radiocobalt activity content and ⁶¹Cu purity of [⁶¹Cu]CuCl₂ solution produced by ^{nat}Ni as target metal for a 50 μA, 3 h deuteron irradiation after FASTlab purification are presented.

TABLE 7
Natural Ni/Nb Target Coin- Analysis of ⁶¹Cu activity
and purity in produced [⁶¹Cu]CuCl₂ solution.

| | Co | | | | | |
|---|---|---|---|---|---|---|
| | species | | | | | |
| | activity in | | | | | |
| Hours | Cu | | | | % Purity PET | |
| post | fraction | ⁶¹Cu activity | ⁶⁴Cu activity | % Purity | nuclides | % non-Cu |
| EoB | [Bq] | [MBq] | [MBq] | ⁶¹Cu | ⁶¹Cu + ⁶⁴Cu | radionuclides |
| 0 | 213864 | 4622 | 70 | | 99.995% | 0.00456% |
| 1 | 213784 | 3756 | 66 | 98.261% | 99.994% | 0.00559% |
| 2 | 213704 | 3052 | 63 | 97.979% | 99.993% | 0.00686% |
| 3 | 213624 | 2479 | 59 | 97.652% | 99.992% | 0.00841% |
| 4 | 213544 | 2015 | 56 | 97.274% | 99.990% | 0.01031% |
| 5 | 213465 | 1637 | 53 | 96.837% | 99.987% | 0.01263% |
| 6 | 213385 | 1330 | 50 | 96.332% | 99.985% | 0.01545% |
| 7 | 213305 | 1081 | 48 | 95.750% | 99.981% | 0.01890% |
| 8 | 213225 | 878 | 45 | 95.081% | 99.977% | 0.02309% |
| 9 | 213145 | 714 | 43 | 94.312% | 99.972% | 0.02817% |
| 10 | 213066 | 580 | 41 | 93.432% | 99.966% | 0.03434% |

Less than 0.03% non-Cu radioisotopes (⁵⁶Co and ⁵⁸Co) will be left in the copper fraction, assuming a product expiry time of 8 h post EoB. This value was lower than the limit allowed for Ga-68 cyclotron-produced as found in the Pharmacopeia (*0.1% at expiry for non-Ga radioisotopes):

The ⁶⁴Cu originating from ^{nat}Ni irradiation (content ˜5% at expiry) will be the main impurity, reducing the radioisotopic purity of ⁶¹Cu product at longer irradiation times or shelf-life (illustrated as the grey curve in FIG. 19).

In Table 8: ⁶⁰Ni/Nb Target coin—Analysis of ⁶¹Cu activity and purity after FASTlab purification. FIG. 19 shows the extrapolated radiocobalt activity content and ⁶¹Cu purity of the produced [⁶¹Cu]CuCl₂ solution.

TABLE 8
⁶⁰Ni/Nb Target coin - Analysis of ⁶¹Cu activity
and purity in produced [⁶¹Cu]CuCl₂ solution.

| | Co species | | | | % Purity | |
|---|---|---|---|---|---|---|
| Hours | activity in Cu | ⁶¹Cu | ⁶⁴Cu | % | PET | |
| post | fraction | activity | activity | Purity | nuclides | % non-Cu |
| EoB | [Bq] | [MBq] | [MBq] | ⁶¹Cu | ⁶¹Cu + ⁶⁴Cu | radionuclides |
| 0 | 243275 | 17498 | 0.378 | | 99.999% | 0.00139% |
| 1 | 243176 | 14217 | 0.358 | 99.996% | 99.998% | 0.00171% |
| 2 | 242977 | 11552 | 0.339 | 99.995% | 99.998% | 0.00210% |
| 3 | 242680 | 9386 | 0.321 | 99.994% | 99.997% | 0.00259% |
| 4 | 242285 | 7627 | 0.304 | 99.993% | 99.997% | 0.00318% |
| 5 | 241792 | 6197 | 0.288 | 99.991% | 99.996% | 0.00390% |
| 6 | 241201 | 5035 | 0.272 | 99.990% | 99.995% | 0.00479% |
| 7 | 240514 | 4091 | 0.258 | 99.988% | 99.994% | 0.00588% |
| 8 | 239731 | 3324 | 0.244 | 99.985% | 99.993% | 0.00721% |
| 9 | 238853 | 2701 | 0.231 | 99.983% | 99.991% | 0.00884% |
| 10 | 237882 | 2195 | 0.219 | 99.979% | 99.989% | 0.01084% |

Less than 0.01% non-Cu radioisotopes (⁵⁶Co and ⁵⁸Co) were left in the Cu fraction, assuming a product expiry time of 8 h post EoB. This value was ten times lower than the allowed limit for ⁶⁸Ga cyclotron-produced as found in the Pharmacopeia (0.1% at expiry for non-Ga radioisotopes*).

Less than 0.02% ⁶⁴Cu was left in the copper fraction at an expiry time of 8 h post EoB, one hundred times lower than the specification required for ⁶⁸Ga (2% Ga radioisotopes were allowed for ⁶⁸Ga).

Purity of Produced [⁶¹Cu]CuCl₂ from Ni/Nb Target Coins: Comparison with Commercially Available Radionuclides

In Table 9, a comparison of the regulatory specifications on the purity of commercially available radionuclides were given along with the characteristics of the high purity [⁶¹Cu]CuCl₂ produced from deuteron irradiation of natNi/Nb and enriched ⁶⁰Ni/Nb target coin (50 μA, 3 h) after FASTlab purification are presented.

TABLE 9
Comparison between commercially available radionuclides and [⁶¹Cu]CuCl₂
solution produced from irradiation of ^{nat}Ni/Nb coins and enriched ⁶⁰Ni/Nb coins.

| | | % Max | | | | |
|---|---|---|---|---|---|---|
| | | radioisotopes | | | | |
| | | of same | % Max other | | | % Max other |
| | % Purity at | element at | radioisotopes | Dominant | % Purity at | radioisotopes |
| Radionuclide | calibration | calibration | at calibration | impurities | expiry | at expiry |
| ¹¹¹In | 99.93% | 0.075% | | ⁶⁵Zn, ^{114 m}In | 99.85% | 0.15% |
| ¹⁸F | | | | ⁵⁶Co | 99.90% | 0.10% |
| ¹⁸F | | | | ⁵⁶Co | 99.99% | 0.01% |
| ⁸Ga cyclotron | 98% | 2% | 0.10% | | | |
| ⁶⁸Ga generator | 99.90% | | 0.001% | ⁶⁸Ge | | |
| ¹⁷⁷Lu | 99.90% | 0.05% | | | | |
| ⁶¹Cu from | 97.27% | 3.16% | 0.013% | ⁵⁶Co, ⁵⁸Co | 95.08% | 5% |
| ^{nat}Ni | (EoB + 4 h) | | | | (EoB + 8 h) | |
| ⁶¹Cu from ⁶⁰Ni | 99.99% | 0.009% | 0.004% | ⁵⁶Co, ⁵⁸Co | 99.98% | 0.02% |
| | (EoB + 4 h) | | | | (EoB + 8 h) | |

As the first notable comparison, cyclotron production of ⁶⁸Ga from proton irradiation also produces long lived radionuclides, (see, e.g., Applied Radiation and Isotopes, 65(10), 1101-1107, IAEA-TECDOC-1 863 Gallium-68 Cyclotron Production)-notably ⁶⁵Zn (half-life=244 days) from the ⁶⁶Zn(p,pn)⁶⁵Zn decay. With a roughly 0.36500 of ⁶⁶Zn in an enriched ⁶⁸Zn starting target metal, about 770 Bq of ⁶⁵Zn will be produced from a 50 μA, 3 h beam with an energy of 13 MeV in a thick target (TENDL-2019 based calculations). Using natural Zn with 27.7% abundancy in ⁶⁶Zn, 58 kBq of ⁶⁵Zn will be produced in one run of 50 μA for 3 h beam. The isotopic purity of Zn in the target metal is, thus, very important.

Similar with [⁶¹Cu]CUCl₂ production, cyclotron production of [⁶⁴Cu]CUCl₂ from proton irradiation also produces long-lived cobalt radionuclides, namely, ⁵⁵Co, ⁵⁷Co, ⁵⁸Co, and ⁶⁰Co. (See, e.g., Nuclear Medicine & Biology, Vol. 24, pp. 35-43, 1997; Applied Radiation and Isotopes 68 (2010) 5-13) By operating with a degraded beam of below 13 MeV, ⁶⁰Co (from ⁶⁴Ni(p,na)⁶⁰Co) was reduced to 1 Bq per run of 50 μA, 3 h. With beam energies below 13 MeV, ⁵⁵Co, formed from the ⁵⁸Ni(p,a)⁵⁵Co reaction, will remain the main impurity (half-life=17.53 hours). The 170 Bq of the long-lived ⁵⁷Co was formed in about 170 Bq in these conditions mostly from ⁶⁰Ni(p,a)⁵⁷Co.

Note: These estimates were computed from thick target yields using TENDL-2019 cross section data and isotopic abundancy of enriched ⁶⁴Ni as follows: 0.00376% ⁵⁸Ni, 0.00298% ⁶⁰Ni, 0.0058%⁶ 6Ni, 0.135% ⁶²Ni, 99.858% ⁶⁴Ni).

### 5. Conclusion

The experimental activities of ⁶¹Cu produced after deuteron irradiation are about 80% of the theoretical yield as calculated from TENDL-2019 cross section data.

The main long-lived nuclides in the radioactive waste fraction from cyclotron production of ⁶¹Cu are radiocobalt species of ⁵⁶Co, ⁵⁷Co, ⁵⁸Co, and ⁶⁰Co. After four years, ⁵⁶Co, ⁵⁷Co, and ⁵⁸Co are calculated to have decayed below regulatory clearance limits, LL*, leaving only ⁶⁰Co. *Clearance limits (LL) means the value corresponding to the specific activity level of a material below which handling of this material is no longer subject to mandatory licensing or supervision.

Target coins with 99% enriched ⁶⁰Ni or ⁶¹Ni improved the yield and purity of the ⁶¹Cu product. Using these targets, the extrapolated purity of ⁶¹Cu was higher as ⁶⁴Cu was not formed as a radioisotopic impurity. Additionally, the ⁵⁶Co and ⁶⁰Co contents were reduced by a factor of 100. ⁵⁷Co amounts increased (but were in low activity), and ⁵⁸Co amounts increased (but 1 decay below LL before ⁵⁶Co/⁵⁸Co).

### Example 5: Radiolabeling

[⁶¹Cu]Cu-NODAGA-1 and [⁶¹Cu]Cu-NODAGA-3

An aliquot of conjugate (3-6 nmol, 1 mg/mL in water) was diluted in 0.25-0.30 mL of ammonium (or sodium) acetate (0.5 M pH 8), followed by the addition of 0.1-0.7 mL [⁶¹Cu]CuCl₂ in 0.05 M HCl (70-240 MBq). The reaction mixture was incubated for 15 min at room temperature (approx. 20-25° C.). The pH of the reaction was between 5 and 6. Quality control was performed on a reverse-phase high performance liquid chromatography (RP-HPLC) connected to a radio-detector (radio-HPLC). Phenomenex Jupiter Proteo C12 (90 Å, 250×4.6 mm) column using the gradient 15-80% B in 8 min (A=H₂O [0.10% TFA], B=ACN [0.1% TFA]) with a flow rate of 1 mL/min. The results of the radio-HPLC are provided in Table 10 below.

[⁶¹Cu]Cu-NODAGA-2 and [⁶¹Cu]Cu-NODAGA-4

⁶¹Cu-labeled conjugates were prepared by incubating 1.5-3 nmol of the corresponding conjugate (as a 1 mg/mL solution) in 125-300 μL of ammonium acetate (0.5 M, pH 8) with 50-200 μL of [⁶¹Cu]CuCl₂ in 0.05 M HCl (33-70 MBq). A pH check was performed in order to guarantee the necessary conditions for the reaction (pH≥5). The reaction mixture was incubated for 10 min at room temperature. Quality control and stability studies were performed by Radio-HPLC on a Shimadzu SCL-40 connected to a GABI radioactivity-HPLC-flow-monitor 7-spectrometer (Elysia-raytest, Straubenhardt, Germany). Radioligands were analyzed using Phenomenex Jupiter Proteo C12 (90 Å, 250×4.6 mm) column using the gradient 15-80% B in 8 min (A=H₂O [0.1% TFA], B=ACN [0.1% TFA]) with a flow rate of 1 mL/min. The results are shown in Table 10.

TABLE 10
Radiochemical purity and retention time
(tR) of the ⁶¹Cu-labeled conjugates

| Radiotracer | Radiochemical purity | t_{R} (min) |
|---|---|---|
| [⁶¹Cu]Cu-NODAGA-1 | ≥98% | 5.9 ± 0.2 |
| [⁶¹Cu]Cu-NODAGA-2 | ≥98% | 6.1 ± 0.2 |
| [⁶¹Cu]Cu-NODAGA-3 | ≥97% | 5.7 ± 0.2 |
| [⁶¹Cu]Cu-NODAGA-4 | ≥98% | 6.4 ± 0.2 |
| [⁶¹Cu]Cu -NODAGA-FAPI-46 | ≥95% | 5.7 ± 0.2 |

All conjugates were labeled with ⁶¹Cu in very high yield and purity. No further purification step was necessary to remove uncomplexed ⁶¹Cu from the reaction mixture, allowing direct us e of the formed radiotracer.

### Example 6: Partition Coefficient (Log D)

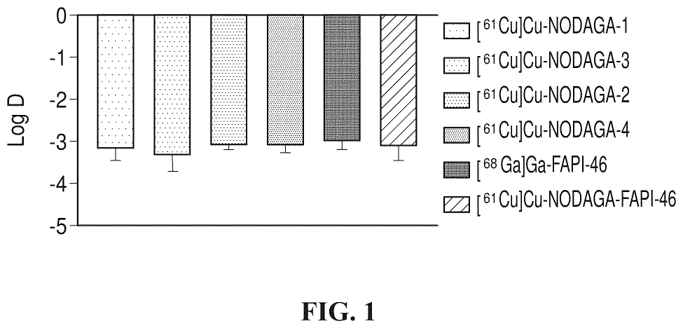

The lipophilic/hydrophilic character of the radiotracers was assessed by the determination of the distribution coefficient (D), expressed as log D (pH=7.4), between an aqua and an organic phase following the “shake-flask” method. In a pre-lubricated Eppendorf tube, a pre-saturated mixture of 500 μL of 1-octanol and 500 μL of PBS pH 7.4 (phosphate-buffered saline) were added. An aliquot of 10 μmol in 10 μL of the radioligand was added to this mixture, shaken for 30 min, and then centrifuged at 3000 rcf for 10 min to achieve phase separation. Aliquots of 100 μL were removed from the 1-octanol and from the PBS phases, and the activity was measured in a γ-counter. The partition coefficient was calculated as the average log ratio value of the radioactivity in the organic fraction and PBS fraction. The results are presented in Table 11 and in FIG. 1.

TABLE 11
Lipophilicity expressed as the log distribution coefficient
D (log D_{O/PBS pH 7.4}) of ⁶¹Cu-labeled conjugates versus
⁶⁸Ga-labeled conjugates (reference radiotracers).

| Radiotracer | log D_{(O/PBS pH 7.4)} |
|---|---|
| [⁶¹Cu]Cu-NODAGA-1 | −3.17 ± 0.28 |
| [⁶¹Cu]Cu-NODAGA-3 | −3.32 ± 0.39 |
| [⁶¹Cu]Cu-NODAGA-2 | −3.09 ± 0.08 |
| [⁶¹Cu]Cu-NODAGA-4 | −3.12 ± 0.16 |
| [⁶¹Cu]Cu-NODAGA-FAPI-46 | −3.10 ± 0.34 |
| [⁶⁸Ga]Ga-FAPI-46 | −3.01 ± 0.18 |

Results are means ± standard deviation from a minimum of two separate experiments, each in triplicates.

### Example 7: In Vitro hFAP Inhibition Assay

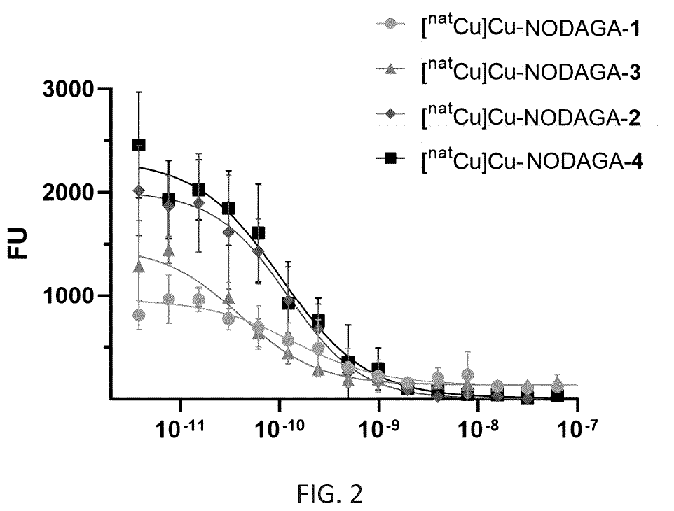

The enzymatic activity of hFAP on the substrate Z-Gly-Pro-AMC was measured at room temperature on a microtiter plate reader, monitoring the fluorescence at an excitation wavelength of 360 nm and an emission wavelength of 465 nm. The assay was performed by mixing the substrate (20 μM), hFAP (200 pM, constant), and the inhibitors in assay buffer (50 mM Tris, 1 M NaCl, 1 mg/mL BSA, pH=7.5), with serial dilution of the inhibitors ranging from 250 nM to 2 fM, 1:2 in a total volume of 20 μL. FAPI-46 was used as positive control. Experiments were performed in triplicate, and the mean fluorescence values were fitted using Graph Pad Pri-sm 9 (equation used: Y=Bottom+(Top−Bottom)/(1+((X\hat{ }HillSlope)/(IC50\hat{ }HillSlope)))). The IC50 value is defined as the concentration of inhibitor required to reduce the enzyme activity by 50% after the addition of the substrate. The results are presented in Table 12 and FIG. 2.

TABLE 12
In Vitro Inhibition Assay

| Compound | IC₅₀ (pM) | 95% CI (pM) |
|---|---|---|
| [^{nat}Cu]Cu- NODAGA-1 | 141.3 | 71.9 to 230.0 |
| [^{nat}Cu]Cu-NODAGA-3 | 40.1 | 26.2 to 54.4 |
| [^{nat}Cu]Cu-NODAGA-2 | 120.5 | 88.0 to 157.8 |
| [^{nat}Cu]Cu-NODAGA-4 | 105.1 | 63.3 to 149.4 |

### Example 8: In Vitro Cellular Uptake

⁶¹Cu-NODAGA-1 and ⁶¹Cu-NODAGA-3

The cellular uptake was studied in vitro using intact cells seeded in 6-well plates overnight. On the day of the experiment, the cells were washed and incubated with each ⁶¹Cu-labeled conjugate at different time points, either alone or in the presence of a blocking agent to distinguish between specific and non-specific uptake. At each investigated time point, the medium containing the unbound (free) radiotracer was removed, followed by two washing steps with ice-cold phosphate-buffered saline. The cells were then treated 2×5 min with ice-cold glycine solution (0.05 M, pH 2.8) to detach the cell surface-bound radiotracer (acid released). Afterwards, the cells containing the internalized radiotracer were detached with 1 M NaOH at 37° C. and collected for measurement. The amount of specific cell surface-bound and internalized radiotracer is expressed as percentage of the total applied activity, after subtracting the non-specific values. [⁶¹Cu]Cu-NODAGA-1, [⁶¹Cu]Cu-NODAGA-3 and [⁶¹Cu]Cu-NODAGA-FAPI-46 (0.2 nM) were assessed in HT-1080.hFAP (FAP-positive) and HT-1080.wt (FAP-negative) cells. Internalization and cell surface-bound fractions for the tested radiotracers are reported in Table 13. The values are expressed as % of the applied activity and refer to the specific uptake calculated after subtracting the non-specific values (measured in the presence of the non-FAP expressing cell line HT-1080.wt) from the total values (specific=total−non-specific).

TABLE 13
Cellular uptake and distribution

| Time | [⁶¹Cu]Cu- | [⁶¹Cu]Cu- | [⁶¹Cu]Cu- | [⁶¹Cu]Cu- | [⁶¹Cu]Cu- |
|---|---|---|---|---|---|
| Point | NODAGA- | NODAGA- | NODAGA- | NODAGA- | NODAGA- |
| [min] | 1 | 2 | 3 | 4 | FAPI-46 |

Cell surface fraction

| | | | | | |
|---|---|---|---|---|---|
| 15 | 1.2 ± 0.3 | 1.2 ± 0.6 | 0.9 ± 0.3 | 1.0 ± 0.7 | 0.9 ± 0.3 |
| 60 | 1.4 ± 0.3 | 1.4 ± 0.5 | 1.2 ± 0.4 | 1.4 ± 0.4 | 1.2 ± 0.4 |
| 240 | 1.3 ± 0.2 | 1.3 ± 0.5 | 1.4 ± 0.4 | 0.9 ± 0.6 | 1.7 ± 0.4 |

Internalized fraction

| | | | | | |
|---|---|---|---|---|---|
| 30 | 26.2 ± 3.5 | 20.7 ± 5.5 | 26.6 ± 4.9 | 22.2 ± 7.4 | 24.3 ± 2.3 |
| 60 | 29.9 ± 1.8 | 27.0 ± 6.9 | 36.9 ± 5.3 | 22.2 ± 5.1 | 36.1 ± 1.6 |
| 240 | 29.3 ± 1.8 | 28.3 ± 7.5 | 39.4 ± 4.8 | 17.4 ± 4.4 | 50.0 ± 6.0 |

[⁶¹Cu]Cu-NODAGA-2 and [⁶¹Cu]Cu-NODAGA-4

Upon thawing, HT-1080.hFAP (FAP-positive), HT-1080.wt (FAP-negative), HEK-293.hFAP and HEK-293.wt cells were kept in culture in MEM medium supplemented with fetal bovine serum (10%, FBS) and Penicillin-Streptomycin (1%) at 37° C. and 5% CO₂. For passaging, cells were detached using Trypsin-EDTA 0.05% when reaching 90% confluency and re-seeded at a dilution of 1:4/1:12 (HT-1080) or 1:10/1:20 (HEK-293).

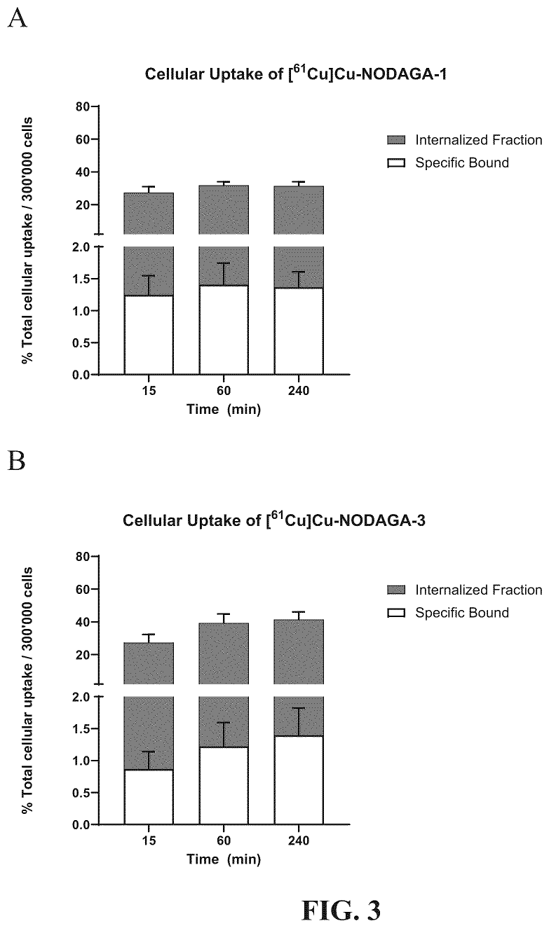

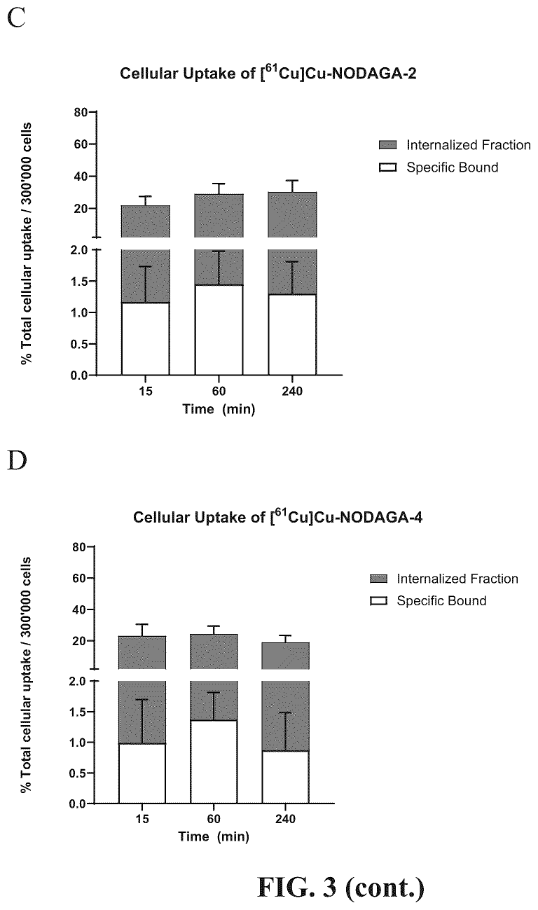

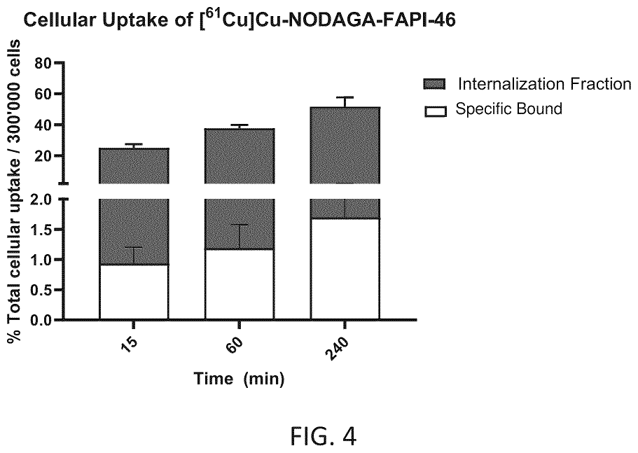

HT-1080.hFAP and HT-1080.wt cells were seeded in a 24-well plate at a concentration of 1.8×10⁵ cells/well in 400 μL of medium 24 hours before the experiment. The cells were then preconditioned in 360 μL of assay medium (MEM medium without supplements) at 37° C. for 60 min. 40 μL of a 2 nM solution of ⁶¹Cu-labeled radioligand was added and the cells were incubated at 37° C. The cellular uptake was interrupted at different time points (15 min, 1 hour and 4 hours), by washing twice with ice-cold PBS. Cell surface-bound radioligand was obtained by washing cells twice with ice-cold glycine buffer (pH 2.8), followed by a collection of the internalized fraction with 1 M NaOH. The activity in each fraction was measured in a γ-counter (Cobra II). The results are expressed as a percentage of the applied radioactivity, after subtracting the non-specific uptake in the HT-1080.wt cells (FIGS. 3 and 4).

The ⁶¹Cu-labeled FAP radiotracers were fast and almost entirely internalized on cell expressing the human FAP at 37° C., with only a negligible amount remaining on the cell surface (cell membrane).

### Example 9: Saturation Binding Experiment

Cell Membrane Preparation: HEK-293.hFAP cells were grown to confluence, mechanically disaggregated, washed with PBS (pH 7.4) and re-suspended in 20 mM of homogenization Tris buffer (pH 7.5) containing 1.3 mM EDTA, 0.25 M sucrose, 0.7 mM bacitracin, 5 μM soybean trypsin inhibitor, and 0.7 mM PMSF. The cells were homogenized using Ultra-Turrax, and the homogenized suspension was centrifuged at 500×g for 10 min at 4° C. The supernatant was collected in centrifuge tubes (Beckman Coulter Inc., Brea, CA, USA). This procedure was then repeated 5 times. The collected supernatant was centrifuged in an ultra-centrifuge (Beckman) at 4° C. for 55 min at 49,000×g. Then, the pellet was re-suspended in 10 mM ice-cold HEPES buffer (pH 7.5), aliquoted, and stored at −80° C. The protein concentration of those membrane suspensions was determined by the Bradford method, BSA as the standard.

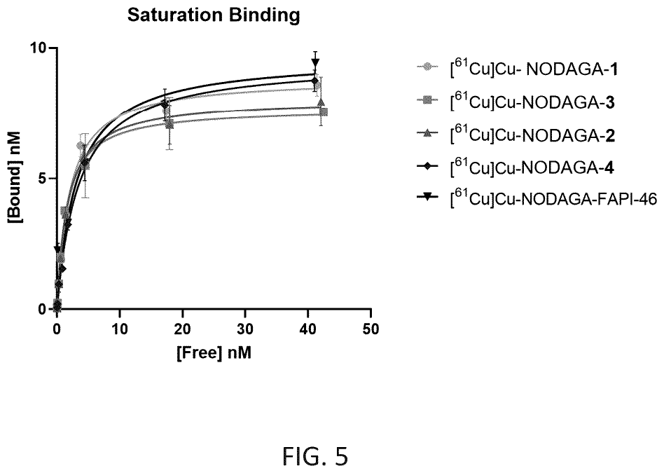

Saturation Experiment: The association profiles of ⁶¹Cu-labeled radioligands were studied at different concentrations, ranging from 0.075 to 50 nM, in HEK-293.hFAP cell membranes at 37° C. Each assay tube contained 170 μL of binding buffer (20 mM HEPES, pH 7.4, containing 4 mM MgCl2, 0.2% BSA, 20 mg/L bacitracin, 20 mg/L PMSF and 200,000 KIU/L aprotinin). The incubation was initiated by adding 30 μL of radioligand solution at 10 times the final concentration and 100 μL of cell membrane suspension to yield 10 μg of protein per well. For the determination of the non-specific binding, 140 μL of the above binding buffer was added along with 30 μL of FAPI-46 to obtain (0.1 mM). Bound fractions were plotted versus the corresponding radioligand concentration at equilibrium. The dissociation constant (KD) and maximal binding capacity (Bmax) values were calculated using GraphPad Software Inc., Prism 7, San Diego, CA, USA (Table 14 and FIG. 5).

TABLE 14
In Vitro Saturation Binding

| Compound | Bₘₐₓ | K_{D} (nM) |
|---|---|---|
| [⁶¹Cu]Cu-NODAGA-1 | 8.6-9.2 | 1.7-2.2 |
| [⁶¹Cu]Cu-NODAGA-3 | 7.3-8.1 | 1.2-1.8 |
| [⁶¹Cu]Cu-NODAGA-2 | 7.7-8.4 | 1.4-2.0 |
| [⁶¹Cu]Cu-NODAGA-4 | 9.1-9.8 | 3.0-3.9 |
| [⁶¹Cu]-NODAGA-FAPI-46 | 9.0-10.3 | 2.3-3.8 |

### Example 10: Mice Studies

All animal experiments were conducted in accordance with Swiss animal welfare laws and regulations under the license number 30515 granted by the Veterinary Office (Department of Health) of the Canton Basel-Stadt.

Tumor Implantation: Female athymic nude-Foxn1nu/Foxn1+ mice (Envigo, The Netherlands), 4-6 weeks old, were injected subcutaneously with 5-10×10⁶ of HT-1080.hFAP cells suspended in 100 μL of PBS on the right shoulder or on the right flank, while 5-10×10⁶ HT-1080.wild-type cells suspended in 100 μL of PBS were injected on the contralateral shoulder or flank. The tumors were allowed to grow to an average volume of 100-200 mm³.

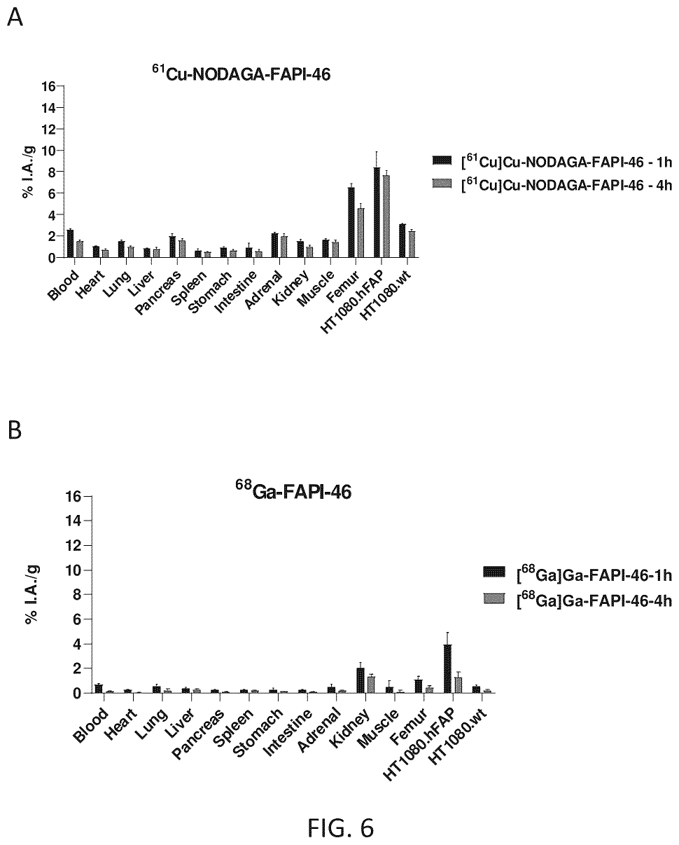

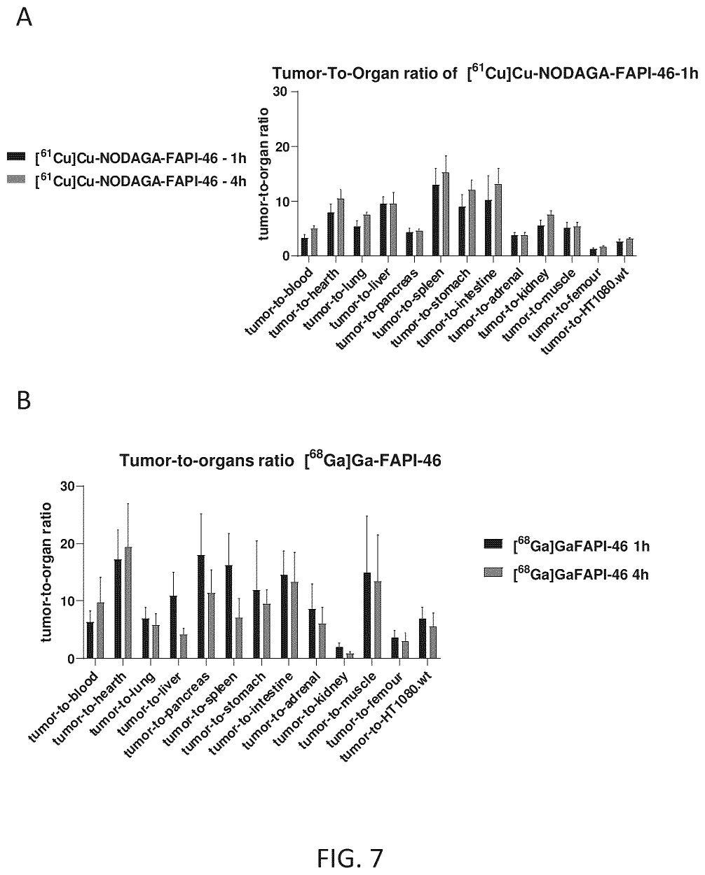

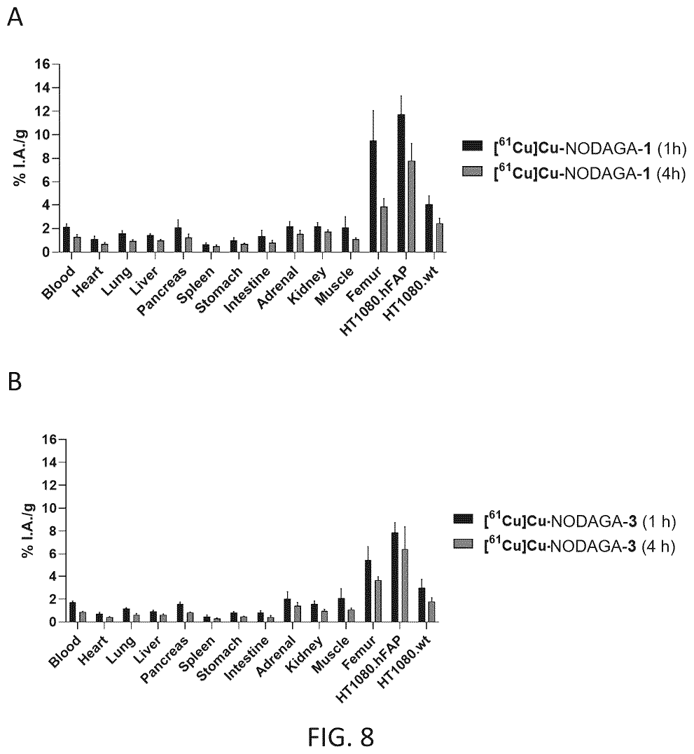

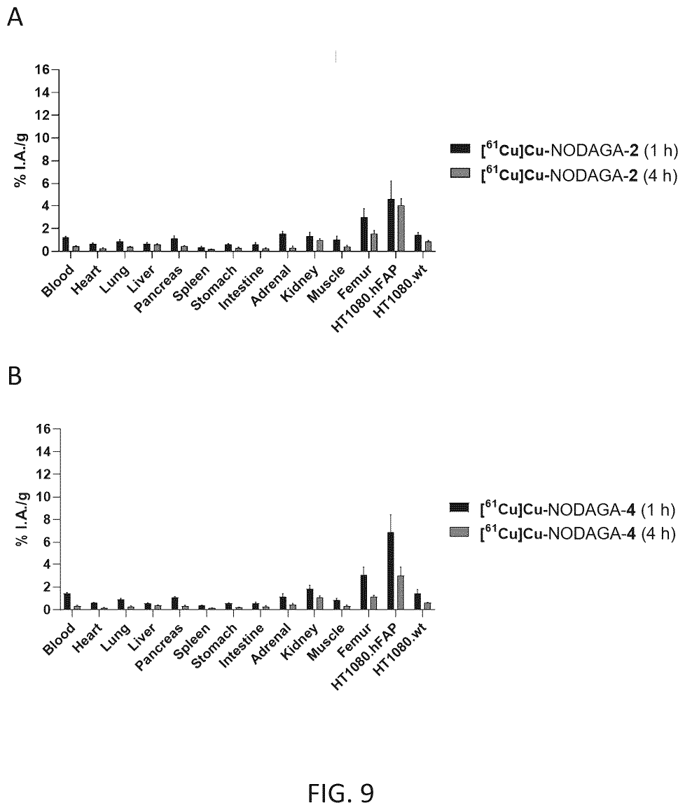

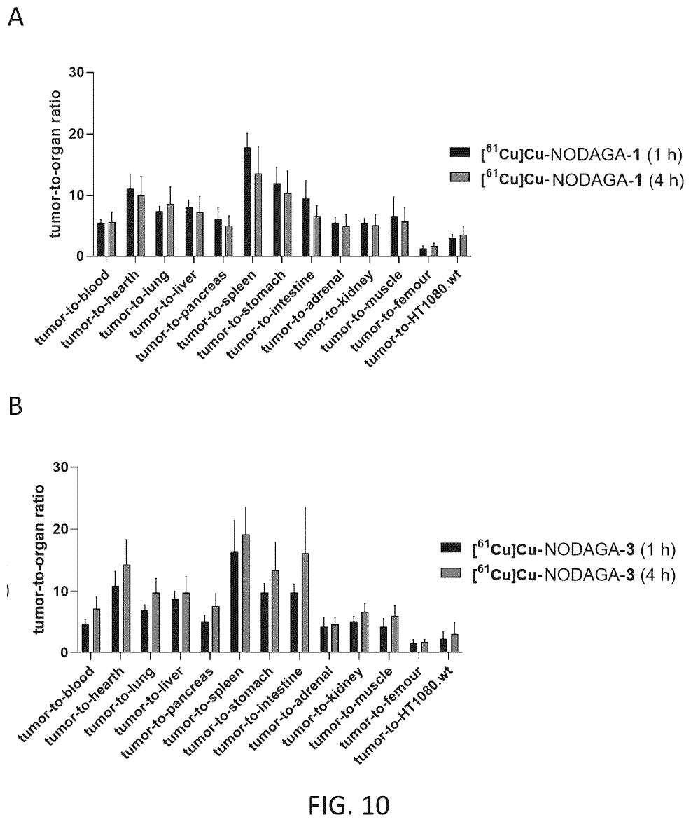

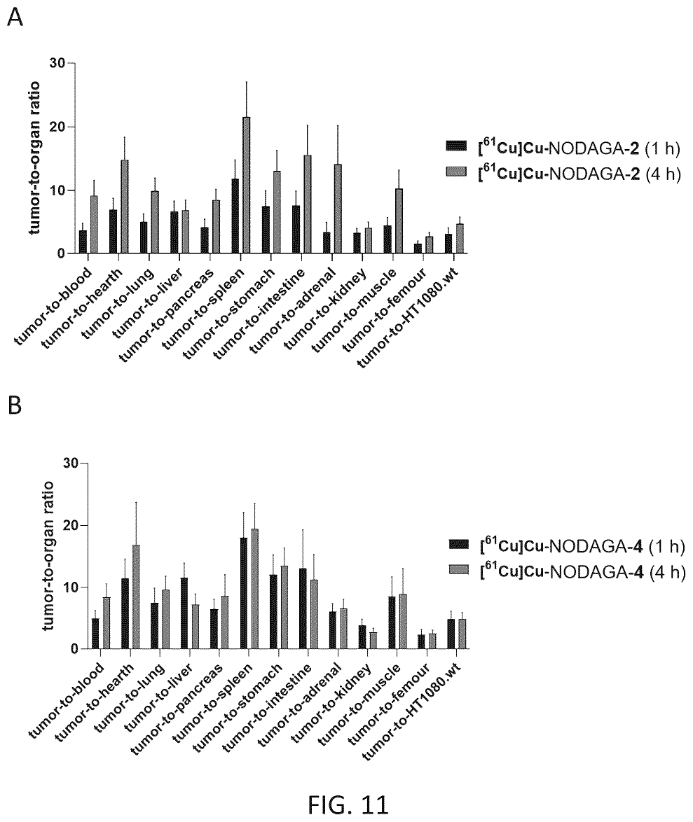

Biodistribution Studies: The xenografted mice were randomized (n=5 per group) and injected intravenously via the tail vein with the ⁶¹Cu-labeled radioligands (100 μL, 500 μmol, 0.8-1 MBq). Mice were euthanized 1 h and 4 h p.i. by C₀₂ asphyxiation. Organs of interest and blood were collected, rinsed of excess blood, blotted dry, weighed, and counted in a γ-counter. The samples were counted against a suitably diluted aliquot of the injected solution as the standard and the results are expressed as the percentage of the injected activity per gram of tissue (% I.A./g) SD. Results are shown in Table 15A-B and FIGS. 6-11.

TABLE 15A
Biodistribution data

| | [⁶¹Cu]Cu-NODAGA-1 | | [⁶¹Cu]Cu-NODAGA-2 | |
|---|---|---|---|---|
| Organ | 1 hour | 4 hours | 1 hour | 4 hours |
| Blood | 2.3 ± 0.1 | 1.2 ± 0.3 | 1.2 ± 0.1 | 0.5 ± 0.1 |
| Heart | 1.2 ± 0.3 | 0.7 ± 0.1 | 0.6 ± 0.1 | 0.3 ± 0.0 |
| Lung | 1.8 ± 0.0 | 0.8 ± 0.2 | 0.8 ± 0.1 | 0.4 ± 0.0 |
| Liver | 1.5 ± 0.0 | 1.0 ± 0.2 | 0.6 ± 0.1 | 0.6 ± 0.1 |
| Pancreas | 2.5 ± 0.3 | 1.4 ± 0.2 | 1.1 ± 0.2 | 0.5 ± 0.0 |
| Spleen | 0.8 ± 0.1 | 0.5 ± 0.1 | 0.4 ± 0.1 | 0.2 ± 0.0 |
| Stomach | 1.1 ± 0.2 | 0.7 ± 0.1 | 0.6 ± 0.1 | 0.3 ± 0.1 |
| Intestine | 1.7 ± 0.4 | 1.1 ± 0.4 | 0.5 ± 0.1 | 0.3 ± 0.1 |
| Adrenal | 2.3 ± 0.5 | 1.4 ± 0.2 | 1.5 ± 0.3 | 0.3 ± 0.1 |
| Kidneys | 2.4 ± 0.3 | 1.5 ± 0.5 | 1.1 ± 0.1 | 1.0 ± 0.1 |
| Muscle | 2.6 ± 0.7 | 1.3 ± 0.1 | 0.9 ± 0.1 | 0.4 ± 0.1 |
| Femur | 10.9 ± 1.1 | 4.2 ± 1.5 | 2.4 ± 0.3 | 1.6 ± 0.3 |
| HT- | 12.6 ± 1.5 | 7.1 ± 3.0 | 3.5 ± 1.0 | 4.0 ± 0.6 |
| 1080.hFAP | | | | |
| HT- | 4.5 ± 0.6 | 2.1 ± 0.5 | 1.4 ± 0.2 | 0.9 ± 0.1 |
| 1080.wt | | | | |
| Tumor | 0.1 ± 0.0 | 0.2 ± 0.2 | 0.4 ± 0.1 | 0.3 ± 0.1 |
| mass | | | | |

TABLE 15B
Biodistribution data

| | [⁶¹Cu]Cu- | | [⁶¹Cu]Cu- | | | |
|---|---|---|---|---|---|---|
| | NODAGA-3 | | NODAGA-4 | | [⁶¹Cu]Cu-NODAGA-FAPI-46 | |
| Organ | 1 hour | 4 hours | 1 hour | 4 hours | 1 hr | 4 hours |
| Blood | 1.7 ± 0.1 | 0.9 ± 0.0 | 1.4 ± 0.1 | 0.4 ± 0.1 | 2.6 ± 0.1 | 1.5 ± 0.1 |
| Heart | 0.7 ± 0.1 | 0.5 ± 0.0 | 0.6 ± 0.0 | 0.2 ± 0.0 | 1.1 ± 0.0 | 0.7 ± 0.1 |
| Lung | 1.2 ± 0.1 | 0.7 ± 0.1 | 0.9 ± 0.1 | 0.3 ± 0.0 | 1.5 ± 0. | 1.0 ± 0.1 |
| Liver | 0.9 ± 0.1 | 0.7 ± 0.1 | 0.6 ± 0.1 | 0.4 ± 0.0 | 0.9 ± 0.0 | 0.8 ± 0.2 |
| Pancreas | 1.6 ± 0.2 | 0.9 ± 0.0 | 1.1 ± 0.1 | 0.4 ± 0.1 | 2.0 ± 0.2 | 1.6 ± 0.1 |
| Spleen | 0.5 ± 0.1 | 0.3 ± 0.0 | 0.4 ± 0.0 | 0.2 ± 0.0 | 0.7 ± 0.1 | 0.5 ± 0.1 |
| Stomach | 0.8 ± 0.1 | 0.5 ± 0.0 | 0.5 ± 0.0 | 0.2 ± 0.0 | 1.0 ± 0.1 | 0.6 ± 0.1 |
| Intestine | 0.8 ± 0.2 | 0.4 ± 0.1 | 0.5 ± 0.2 | 0.3 ± 0.1 | 1.0 ± 0.4 | 0.6 ± 0.2 |
| Adrenal | 2.1 ± 0.6 | 1.4 ± 0.3 | 1.1 ± 0.2 | 0.5 ± 0.1 | 2.2 ± 0.1 | 2.0 ± 0.2 |
| Kidneys | 1.6 ± 0.3 | 1.0 ± 0.2 | 1.8 ± 0.2 | 1.1 ± 0.1 | 1.5 ± 0.1 | 1.0 ± 0.1 |
| Muscle | 2.1 ± 0.8 | 1.1 ± 0.1 | 0.8 ± 0.2 | 0.4 ± 0.1 | 1.7 ± 0.1 | 1.4 ± 0.2 |
| Femur | 5.4 ± 1.2 | 3.7 ± 0.3 | 3.2 ± 0.8 | 1.2 ± 0.1 | 6.5 ± 0.4 | 4.6 ± 0.4 |
| HT- | 7.9 ± 0.9 | 6.4 ± 2.0 | 7.4 ± 1.6 | 3.0 ± 0.8 | 8.4 ± 1.4 | 7.7 ± 0.4 |
| 1080.hFAP | | | | | | |
| HT- | 4.0 ± 2.2 | 1.8 ± 0.3 | 1.7 ± 0.2 | 0.6 ± 0.0 | 3.1 ± 0.0 | 2.5 ± 0.2 |
| 1080.wt | | | | | | |
| Tumor | 0.1 ± 0.0 | 0.1 ± 0.1 | 0.3 ± 0.1 | 0.3 ± 0.1 | 0.1 ± 0.0 | 0.2 ± 0.0 |
| mass | | | | | | |

[⁶¹Cu]Cu-NODAGA-1, [⁶¹Cu]Cu-NODAGA-2, [⁶¹Cu]Cu-NODAGA-3, [⁶¹Cu]Cu-NODAGA-4, and [⁶¹Cu]Cu-NODAGA-1 showed high accumulation in FAP-positive (HT-1080.hFAP) tumor and murine-FAP-positive tissues, such as the bone marrow (bones).

PET/CT Imaging: Mice bearing FAP-positive and FAP-negative xenografts were injected intravenously with ⁶¹Cu-labeled radioligands of the present disclosure or [⁶¹Cu]Cu-NODAGA-FAPI-46 (100 μL/500 μmol/6-12 MBq). Mice were anesthetized with 1.5% isoflurane and dynamic PET scans were acquired during 1 hour upon injection of the radiotracer. The mice were euthanized by CO2 at 4 hours p.i., and static PET scans were acquired for 30 min.

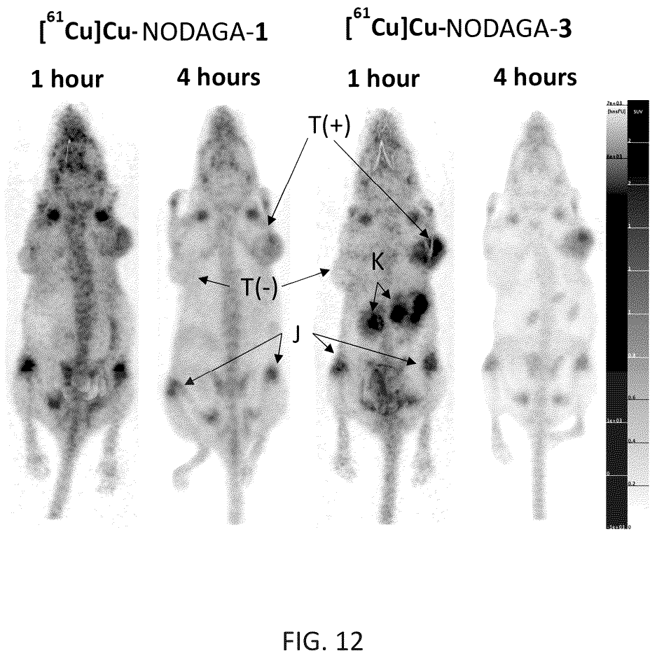

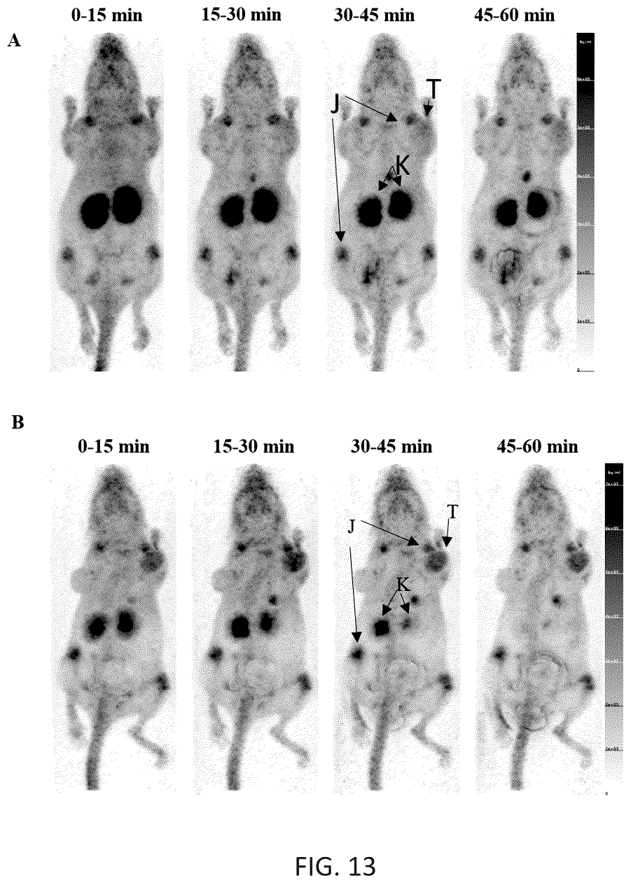

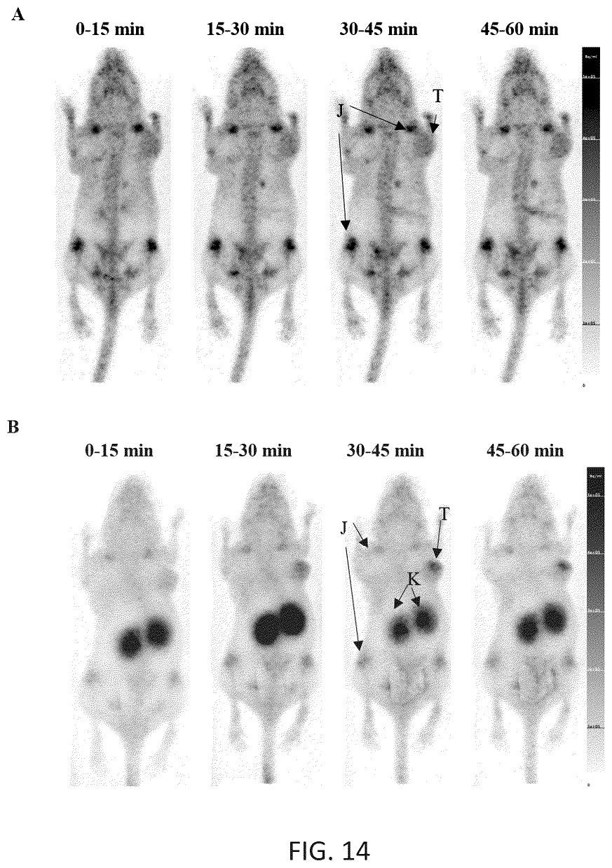

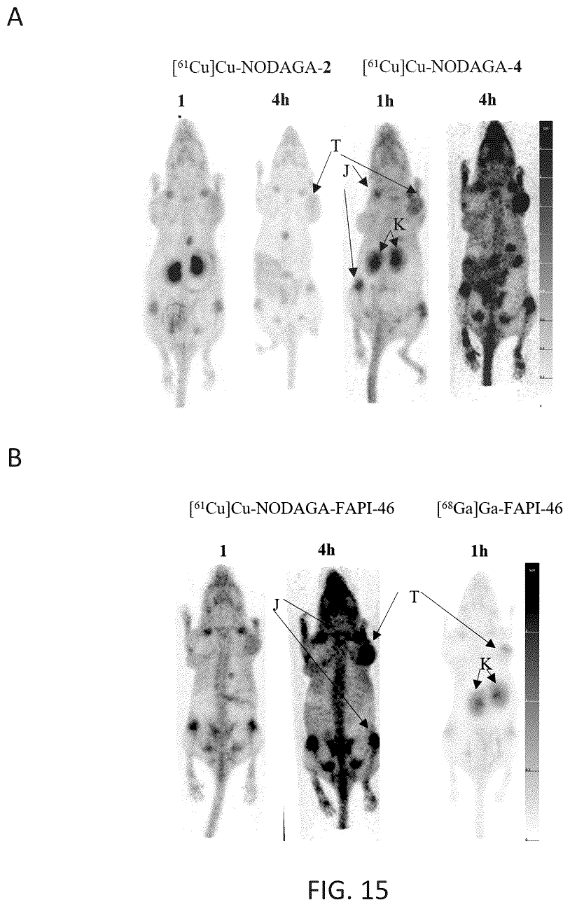

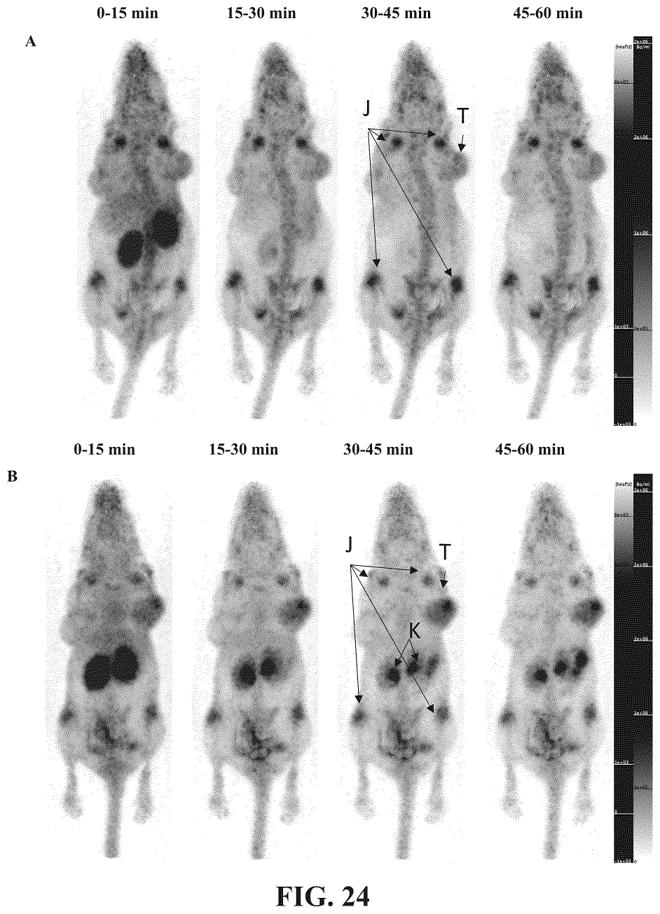

PET/CT images were acquired using β-CUBE PET scanner system (Molecubes, Gent, Belgium), with a spatial resolution of 0.85 mm and an axial field-of-view of 13 cm. Dynamic PET scans were acquired for 60 min. All PET scans were decay corrected and reconstructed into a 192×192×384 matrix by an ordered subsets maximization expectation (OSEM) algorithm using 30 iterations, a voxel size of 400×400×400 μm a 15 min per frame. CT data was used to apply attenuation correction on the PET data. The CT was imaged supine, head first, using the NanoSPECT/CTTM scanner (Bioscan Inc.). Topograms and helical CT scans of the whole mouse were first acquired using the following parameters: X-ray tube current: 177 μA, X-ray tube voltage 45 kVp, 90 seconds and 180 frames per rotation, pitch 1. CT images were reconstructed using CTReco (version r1.146), with a standard filtered back projection algorithm (exact cone beam) and post-filtered (RamLak, 100% frequency cut-off), resulting in a pixel size of 0.2 mm. Co-registered PET/CT images were visualized using maximum intensity projection (MIP) with VivoQuant software (version 4.0). (FIGS. 12-15, and FIG. 24).

Remaining PET activity in the mouse body 4 h p.i. prior to the 4 h scan was determined (Table 16). [⁶¹Cu]Cu-NODAGA-FAPI-46 and [⁶¹Cu]Cu-NODAGA-1 showed the highest retention in the body, while [⁶¹Cu]Cu-NODAGA-4 presented the lowest value. Due to the physical characteristic of the radionuclide, [⁶⁸Ga]Ga-FAPI-46 was not evaluated 4 h p.i.

TABLE 16
In Vitro PET Remaining Activity

| | Injected | Activity left | Percentage |
|---|---|---|---|
| | Activity | after 4 h | of activity |
| | (MBq) | (MBq) | left |
| [⁶¹Cu] Cu-NODAGA-1 | 10.07 | 1.85 | 18.4% |
| [⁶¹Cu]Cu-NODAGA-3 | 7.65 | 0.86 | 11.2% |
| [⁶¹Cu] Cu-NODAGA-2 | 12.12 | 1.31 | 10.8% |
| [⁶¹Cu]Cu-NODAGA-4 | 10.34 | 0.76 | 7.4% |
| [⁶¹Cu]Cu-NODAGA-FAPI-46 | 7.25 | 1.34 | 18.9% |
| [⁶⁸Ga] Ga-FAPI-46 | 12.23 | / | / |

### 7. EQUIVALENTS AND INCORPORATION BY REFERENCE

While the provided disclosure has been particularly shown and described with reference to a preferred embodiment and various alternate embodiments, it will be understood by persons skilled in the relevant art that various changes in form and details can be made therein without departing from the spirit and scope of the provided disclosure.

All references, issued patents, and patent applications cited within the body of the instant specification, are hereby incorporated by reference in their entirety, for all purposes. In particular, U.S. Provisional Patent Application Nos. 63/409,684 (filed Sep. 23, 2022); 63/409,687 (filed Sep. 23, 2022); 63/416,479 (filed Oct. 14, 2022); 63/520,329 (filed Aug. 17, 2023); and 63/520,323 (filed Aug. 17, 2023) are hereby incorporated by reference in their entirety. Additionally, the following U.S. non-provisional patent applications, concurrently filed with the present application, are also incorporated by reference in their entirety:
the application titled “SOLID TARGET SYSTEMS FOR THE PRODUCTION OF HIGH PURITY RADIONUCLIDE COMPOSITIONS” filed Sep. 25, 2023 under attorney docket no. 39973-53109 (001US); and
the application titled “HIGH PURITY COPPER RADIOPHARMACEUTICAL COMPOSITIONS AND DIAGNOSTIC AND THERAPEUTIC USES THEREOF” filed Sep. 25, 2023 under attorney docket no. 39973-52915 (002US).

1.-140. (canceled)

141. A compound, wherein the compound is of Formula I: 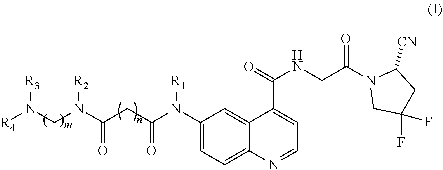
wherein:
R¹ is R^{a};
R² and R³ are each R^{a} or together form a C₂₋₉ heterocycle with the nitrogen atoms to which they are attached;
R⁴ is H, an amine protecting group, or -L-T;
R^{a}, independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl;
L is a bond or a divalent linker;
T comprises (a) a chelating moiety suitable for chelating a radionuclide, (b) an imaging agent, or (c) a drug;
n is an integer from 1 to 20; and
m is an integer from 1 to 20;
or is a pharmaceutically acceptable salt thereof.

142. The compound of claim 141, wherein R¹ is methyl or H.

143. The compound of claim 141, wherein R² is H and R³ is H.

144. The compound of claim 141, wherein R² and R³ together form a C₂₋₉ heterocycle with the nitrogen atoms to which they are attached.

145. The compound of claim 144, wherein the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane.

146. The compound of claim 141, wherein R⁴ is H.

147. The compound of claim 141, wherein R⁴ is an amine protecting group.

148. The compound of claim 141, wherein R⁴ is -L-T.

149. The compound of claim 148, wherein L is a divalent linker selected from an acid-labile linker, a hydrolysis-labile linker, an enzymatically cleavable linker, a reduction labile linker, a self-immolative linker, and a non-cleavable linker.

150. The compound of claim 148, wherein T comprises a chelating moiety suitable for chelating a radionuclide.

151. The compound of claim 150, wherein the chelating moiety is chelated to a radionuclide, and the radionuclide is selected from alpha radiation emitting isotopes, beta radiation emitting isotopes, gamma radiation emitting isotopes, Auger electron emitting isotopes, X-ray emitting isotopes, and fluorescence emitting isotopes.

152. The compound of claim 150, wherein the radionuclide is selected from ²²⁵Ac, ⁵¹Cr, ⁶⁶Ga, ⁶⁷Ga, ⁶⁸GA, [¹⁸F]AlF, ¹¹¹In, ^{113m}In, ^{52m}Mn, ^{99m}Tc, ¹⁸⁶Re, ¹⁸⁸Re, ¹³⁹La, ¹⁴⁰La, ¹⁷⁵Yb, ¹⁷⁹Yb, ¹⁵³Sm, ^{177m}Sn, ¹⁶⁶Ho, ⁸⁶Y, ⁸⁸Y, ⁹⁰Y, ¹⁴⁹Pm, ¹⁶⁵Dy, ¹⁶⁹Er, ¹⁷⁷Lu, ⁵²Fe, ⁴³Sc, ⁴⁴Sc, ⁴⁶Sc, ⁴⁷Sc, ¹⁴²Pr, ¹⁵⁷Gd, ¹⁵⁹Gd, ²¹²Bi, ²¹³Bi, ⁷²As, ⁷⁷As, ⁹⁷Ru, ¹⁰⁹Pd, ¹⁰⁵Rh, ^{101m}Rh, ¹¹⁹Sb, ¹⁹⁷Hg, ¹⁵¹Eu, ¹⁵³Eu, ¹⁶⁹Eu, ²⁰¹Tl, ¹⁴⁹Tb, ¹⁵²Tb, ¹⁵⁵Tb, ¹⁶¹Tb, ²⁰³Pb, ²¹²Pb, ¹⁵¹Pm, ¹⁵³Pm, ¹⁴²Pr, ¹⁴³Pr, ⁵⁵Co, ⁶⁰Cu, ⁶¹Cu, ⁶²Cu, ⁶⁴Cu, ⁶⁷Cu, ⁶²Zn, ¹⁸⁸Re, ¹⁹⁸Au, ¹⁹⁹Au, ²²⁷Th, ¹¹¹Ag, ¹⁹⁹Ag, ²¹¹At, ²²³Ra, ⁸⁸Zr, and ⁸⁹Zr.

153. The compound of claim 152, wherein the radionuclide is selected from ⁶¹Cu, ⁶⁴Cu, and ⁶⁷Cu.

154. The compound of claim 150, wherein the chelating moiety is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate), NODAGA (1,4,7-triazacyclononane-1-glutaric acid-4,7-acetic acid) (also known as NOTAGA), NODA Deferoxamine (1,4,7-triazacyclononane-1,4-diyl)diacetic acid DFO), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), and 4,4′-((3,6,10,13,16,19-hexaazabicyclo[6.6.6]ico-sane-1,8-diylbis(aza-nediyl))bis(methylene))dibenzoic acid (BaBaSar).

155. The compound of claim 148, wherein T comprises an imaging agent, wherein the imaging agent comprises a radionuclide or a fluorescent dye.

156. The compound of claim 148, wherein T comprises a drug, wherein the drug comprises a chelating moiety chelated to a radionuclide.

157. The compound of claim 141, wherein the compound is of Formula Ia or Formula (Ib):

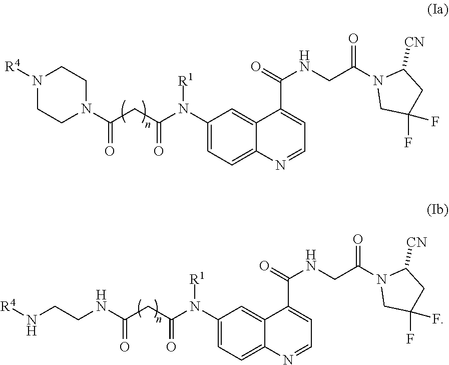

158. The compound of claim 157, wherein R¹ is H or methyl.

159. The compound of claim 157, wherein R⁴ is -L-T.

160. The compound of claim 159, wherein T comprises a chelating moiety chelated to a radionuclide, wherein
the chelating moiety is selected from DOTAGA, DOTA, NOTA, NODAGA, and NODA; and
the radionuclide is selected from ⁶¹Cu, ⁶⁴Cu, and ⁶⁷Cu.

161. The compound of claim 141, wherein the compound is selected from:

| Compound | Structure | |
|---|---|---|
| 1 | 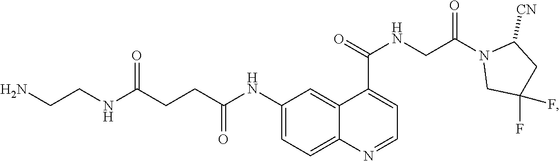 [SMILES: N#C[C@@H]1CC(F)(F)CN1C(=O)CNC(=O)c1ccnc2ccc(NC(=O)CCC(=O)NCCN)cc12] | |
| 2 | 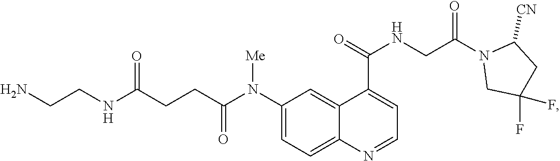 [SMILES: CN(C(=O)CCC(=O)NCCN)c1ccc2nccc(C(=O)NCC(=O)N3CC(F)(F)C[C@H]3C#N)c2c1] | |
| 3 | 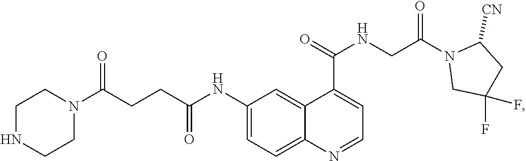 [SMILES: N#C[C@@H]1CC(F)(F)CN1C(=O)CNC(=O)c1ccnc2ccc(NC(=O)CCC(=O)N3CCNCC3)cc12] | |
| 4 | 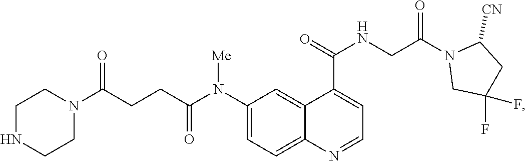 [SMILES: CN(C(=O)CCC(=O)N1CCNCC1)c1ccc2nccc(C(=O)NCC(=O)N3CC(F)(F)C[C@H]3C#N)c2c1] | |
| NODAGA-1 | 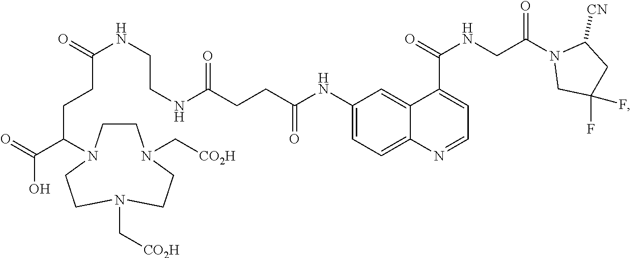 [SMILES: N#C[C@@H]1CC(F)(F)CN1C(=O)CNC(=O)c1ccnc2ccc(NC(=O)CCC(=O)NCCNC(=O)CCC(C(=O)O)N3CCN(CC(=O)O)CCN(CC(=O)O)CC3)cc12] | |
| NODAGA-2 | 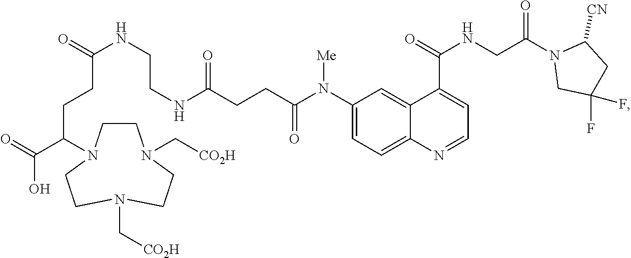 [SMILES: CN(C(=O)CCC(=O)NCCNC(=O)CCC(C(=O)O)N1CCN(CC(=O)O)CCN(CC(=O)O)CC1)c1ccc2nccc(C(=O)NCC(=O)N3CC(F)(F)C[C@H]3C#N)c2c1] | |
| NODAGA-3 | 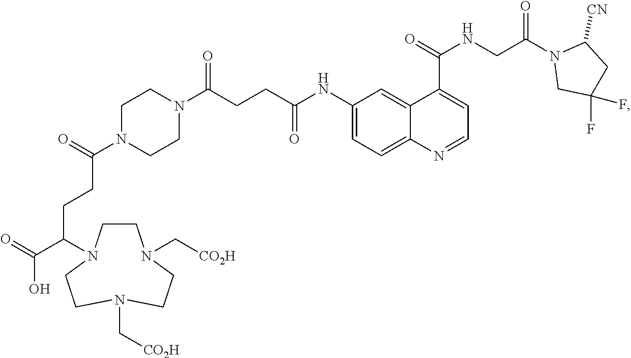 [SMILES: N#C[C@@H]1CC(F)(F)CN1C(=O)CNC(=O)c1ccnc2ccc(NC(=O)CCC(=O)N3CCN(C(=O)CCC(C(=O)O)N4CCN(CC(=O)O)CCN(CC(=O)O)CC4)CC3)cc12] | |
| NODAGA-4 | 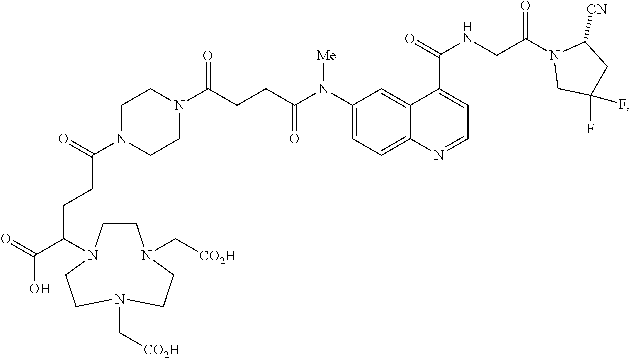 [SMILES: CN(C(=O)CCC(=O)N1CCN(C(=O)CCC(C(=O)O)N2CCN(CC(=O)O)CCN(CC(=O)O)CC2)CC1)c1ccc2nccc(C(=O)NCC(=O)N3CC(F)(F)C[C@H]3C#N)c2c1] | |
| *Cu-NODAGA-1 | 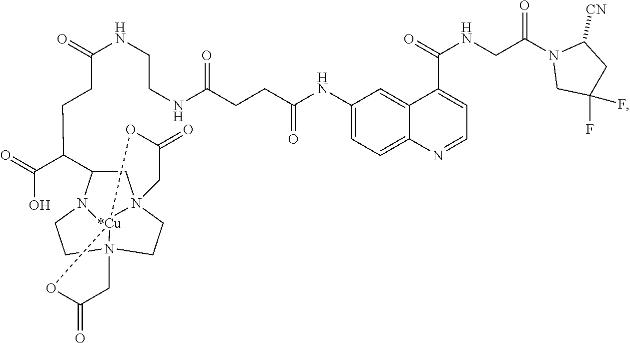 | |
| *Cu-NODAGA-2 | 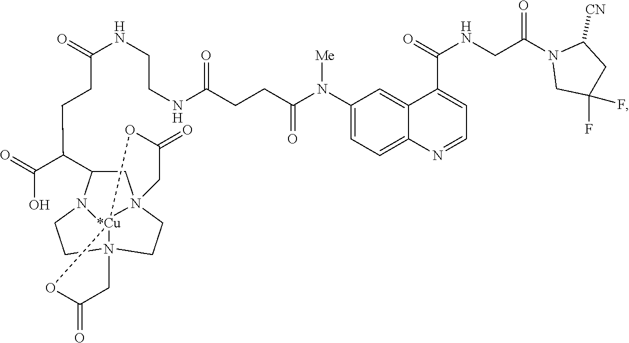 | |
| *Cu-NODAGA-3 | 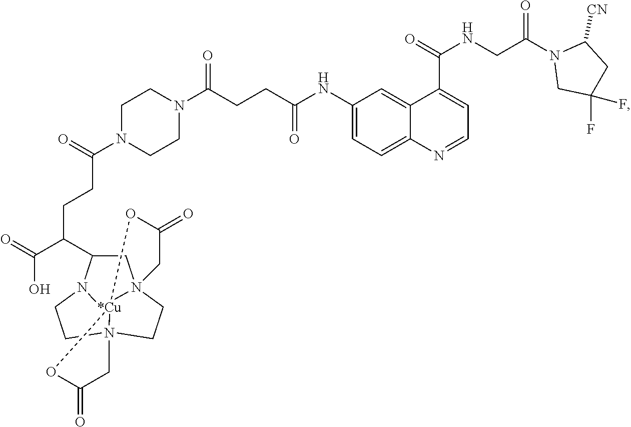 | and |
| | |
|---|---|
| *Cu-NODAGA-4 | 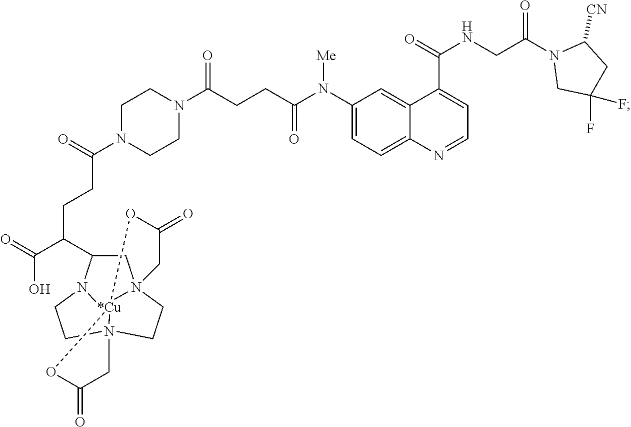 |

or is a pharmaceutically acceptable salt thereof, wherein *Cu is selected from ⁶¹Cu, ⁶²Cu, ⁶⁴Cu, and ⁶⁷Cu.

162. A pharmaceutical composition comprising a compound of claim 141 and a pharmaceutically acceptable excipient.

163. The pharmaceutical composition of claim 162, wherein the composition is characterized by one or more of: (i) molar activity of ≥3 MBq/nmol, (ii) radiochemical purity≥91%, (iii) activity concentration of ≥8 MBq/mL, and (iv) radionuclidic purity of the compound at end of synthesis (EoB plus 2 hours) of ≥9500.

164. A method of generating one or more images of a subject comprising:
administering to a subject an effective amount of a compound of claim 153, wherein the radionuclide is ⁶¹Cu; and
generating one or more images of at least a part of the subject's body.

165. The method of claim 164, wherein the image is generated using positron emission tomography (PET), PET-computer tomography (PET-CT), or single-photon emission computerized tomography (SPECT).

166. A method of treating a disease in a patient in need thereof, comprising administering to the patient an effective amount of a compound of claim 153, wherein the radionuclide is ⁶⁷Cu.

167. The method of claim 166, wherein the disease is selected from cancers, inflammatory diseases, infectious diseases, and immune diseases.

168. A theranostic method comprising:
(a) administering to a subject an effective amount of a first compound or a pharmaceutical composition comprising an effective amount of the first compound;
(b) generating one or more images of the subject; and
(c) administering to the subject an effective amount of a second compound or a pharmaceutical composition comprising an effective amount of the second compound,
wherein the first and second compounds are of claim 153 and differ only in radioisotopic identity, wherein the radionuclide of the first compound is ⁶¹Cu and the radionuclide of the second compound is ⁶⁷Cu.

169. The method of claim 168, wherein:
(a) the first compound is ⁶¹[Cu]Cu-NODAGA-1 and the second compound is ⁶⁷[Cu] Cu-NODAGA-1;
(b) the first compound is ⁶¹[Cu]Cu-NODAGA-2 and the second compound is ⁶⁷[Cu] Cu-NODAGA-2;
(c) the first compound is ⁶¹[Cu]Cu-NODAGA-3 and the second compound is ⁶⁷[Cu] Cu-NODAGA-3; or
(d) the first compound is ⁶¹[Cu]Cu-NODAGA-4 and the second compound is ⁶⁷[Cu] Cu-NODAGA-4.

170. The method of claim 168, further comprising determining, via the one or more images of the subject, the presence or absence of a disease in the subject based on the presence or absence of localization of the ⁶¹Cu radionuclide of the first compound in the subject's body.
